(19) United States
(12) Patent Application Publication
(10) Pub. No.: US 2025/0166731 A1
(43) Pub. Date: May 22, 2025

(54) SYSTEMS AND METHODS FOR GENETIC IMPUTATION, FEATURE EXTRACTION, AND DIMENSIONALITY REDUCTION IN GENOMIC SEQUENCES

(71) Applicant: The Scripps Research Institute, La Jolla, CA (US)

(72) Inventors: Ali Torkamani, San Diego, CA (US); Raquel Dias, Gainesville, FL (US)

(21) Appl. No.: 18/833,305

(22) PCT Filed: Jan. 27, 2023
(86) PCT No.: PCT/US2023/061455

§ 371 Date: Jul. 25, 2024

Related U.S. Application Data
(60) Provisional application No. 63/304,427, filed on Jan. 28, 2022.

(51) Int. Cl. G16B 30/10; G06N 3/0455; G06N 3/048; G06N 3/0495; G06N 3/084; G06N 3/088; G06N 3/0985; G16B 20/20; G16B 20/40

(52) U.S. Cl. CPC G16B 30/10; G06N 3/0455; G06N 3/048; G06N 3/0495; G06N 3/084; G06N 3/088; G06N 3/0985; G16B 20/20; G16B 20/40

(57) ABSTRACT

The invention relates to the use of artificial neural networks, such as autoencoders, in population genomics and individual genome processing to fill-in missing data from genomic assays with significant sparsity, such as low-pass whole genome sequencing or array-based genotyping. An autoencoder-based neural network approach for the simultaneous execution of genetic imputation as well as feature extraction and dimensionality reduction for downstream tasks is provided.

### CLAIM OF PRIORITY UNDER 35 U.S.C. § 119

The present application for patent claims benefit of Provisional Application No. 63/304,427 entitled “A NEURAL NETWORK APPROACH TO GENETIC IMPUTATION, FEATURE EXTRACTION, AND DIMENSIONALITY REDUCTION” filed Jan. 28, 2022 and hereby expressly incorporated by reference herein.

### BACKGROUND

The human genome is inherited in large blocks from parental genomes, generated through a DNA-sequence-dependent shuffling process called recombination. The non-random nature of recombination breakpoints producing these genomic blocks results in correlative genotype relationships across genetic variants, known as linkage disequilibrium. Thus, genotypes for a small subset (1%-10%) of observed common genetic variants can be used to infer the genotype status of unobserved but known genetic variation sites across the genome (on the order of ˜1M of >10M sites)^{1,2}. This process, called genotype imputation, allows for the generation of nearly the full complement of known common genetic variation at a fraction of the cost of direct genotyping or sequencing. Given the massive scale of genotyping required for genome-wide association studies or implementation of genetically-informed population health initiatives, genotype imputation is an essential approach in population genetics.

Standard approaches to genotype imputation utilize Hidden Markov Models (HMM)³⁻⁵ distributed alongside large whole genome sequencing (WGS)-based reference panels⁶. In general terms, these imputation algorithms use genetic variants shared between to-be-imputed genomes and the reference panel and apply HMM to impute the missing genotypes per sample⁷. Genotyped variants are the observed states of the HMM, whereas the to-be-imputed genetic variants present in the reference panel are the hidden states. The HMM algorithm depends on recombination rates, mutation rates, and/or genotype error rates that must be fit by Markov Chain Monte Carlo Algorithm (MCMC) or an expectation-maximization algorithm. Thus, HMM-based imputation is a computationally intensive process, requiring access to both high-performance computing environments and large, privacy-sensitive, WGS reference panels⁸. Often, investigators outside of large consortia will resort to submitting genotype data to imputation servers³, resulting in privacy and scalability concerns⁹.

### SUMMARY

Embodiments of the invention relate to a system for dynamically producing predictive data using varying data. In some embodiments, the system can be structured to generate imputed genomic sequence by inputting actual genomic sequence into an artificial neural network. In some embodiments, the system can include one or more of: an artificial neural network; a communication device; and/or a processing device communicably coupled to the communication device.

The processing device can be configured to: (i) receive actual genetic information; (ii) generate an input vector, wherein the input vector can include probabilistic or binary data converted from allelic data; (iii) access the neural network including a dynamic reservoir containing units, wherein each of the units can be connected to at least one other unit in the dynamic reservoir and the connections between the units are weighted; (iv) input the input vector into the neural network in order to generate an imputed sequence; (vi) generate, via the neural network, an imputed sequence, wherein the imputed sequence is an output of the neural network and at least partially based on the input vector; (vii) modify the initial prediction to generate a final prediction; (viii) present the final prediction to a user; and/or (ix) export the final prediction to a computer system.

In some embodiments, the variables can include data relating to at least one genetic variant.

In some embodiments, the genetic variant can be at least one of: a single-nucleotide variant, a multi-nucleotide variant, an insertion, a deletion, a structural variation, an inversion, a copy-number change, or any other genetic variation relative to a reference genome, or the like.

In some embodiments, the units can include nodes within at least three layers.

In some embodiments, the dynamic reservoir can include a plurality of layers.

In some embodiments, the at least three layers can include an input layer, a hidden layer, and an output layer.

In some embodiments, the system can include a plurality of dynamic reservoirs, wherein each reservoir can be adapted to impute sequence of a selected portion of genetic data, and wherein the plurality of reservoirs can be adapted to cover all desired portions of a genome or fragment thereof.

In some embodiments, the initial prediction can be provided in binary form, and the final prediction can be provided as genetic sequence.

Some embodiments of the invention relate to a method of obtaining, from an input of incomplete genomic information from an individual or population of an organism, an output of more complete genomic information for the individual or population within a desired accuracy cutoff The method can include one or more of the steps of: (a) providing a genetic inference model comprising encoded complex genotype relationships, the model having been encoded by a system described herein, (b) inputting incomplete genomic information from the individual or population into the model in or mediated by the system wherein the incomplete genetic information comprises at least a sparse genotyping or sequencing, as defined and described herein, of a randomly sampled genome of the individual or population; (c) applying the model to the information by operation of the system; and/or (d) obtaining the output of more complete genomic information for the individual or population, wherein the more complete genomic information can include genotypes for genetic variants observed in a reference population used to define the weights of the neural network.

In some embodiments, the accuracy cutoff can be an accuracy level as in any of the ranges depicted in any of the figures, or any other useful accuracy. For example, the accuracy level can be 99.9% or more, 99.5%, 99%, 98%, 97%, 96%, 95%, 92%, 90%, 85%, 80%, 75%, 70%, 65%, or 60%.

In some embodiments, the organism can be selected from an animal, a plant, a fungus, a chromistan, a protozoan, a bacterium, an aracheon, and the like.

Some embodiments of the invention relate to a method of training the system described herein. The method can includes one or more of the steps of: (a) providing training genetic sequence data; (b) generating a first training input vector, wherein the first training input vector can include binary or probabilistic data converted from allelic data of the training genetic sequence data; (c) generating a second training input vector from the first training input vector, wherein the second input vector can include reducing a number of variables of the first training input vector; (d) inputting the second training input vector to a neural network comprising a dynamic reservoir containing units, wherein each of the units can be connected to at least one other unit in the dynamic reservoir and wherein the connections between the units can be initially weighted according to a training weighting; (e) generating, via the neural network, imputed training sequence data, wherein the imputed training sequence data are an output of the neural network and at least partially based on the second input vector; (f) comparing the imputed training sequence data from step (e) to the original training genetic sequence data from step (a) to obtain a training result reflecting a degree of correspondence between the imputed training sequence data and the original training genetic sequence data; (g) modifying the at least one of the generating of step (c) and the weighting of step (d), based upon the training result of step (f); (h) repeating steps (c) through (g) until the correspondence reaches a predetermined value; and thereby (i) completing training of the system.

In some embodiments, the training genetic sequence can include at least one full genomic sequence of an organism.

In some embodiments, the training genetic sequence can include a plurality of full genomic sequences from a selected population of an organism.

### BRIEF DESCRIPTION OF THE DRAWINGS

FIG. 1 depicts a schematic overview of autoencoder training workflow. (A) Tiling of autoencoders across the genome can be achieved by (A.1) calculating a n×n matrix of pairwise SNP correlations, thresholding them at 0.45 (selected values are shown in red background, excluded values in gray), (A.2) quantifying the overall local LD strength centered at each SNP by computing their local correlation box counts and splitting the genome into approximately independent segments by identifying local minima (recombination hotspots). The arrow illustrates minima between strong LD regions. For reducing computational complexity, the correlations were calculated in a fixed sliding box size of 500×500 common variants (MAF≥0.5%). Thus, the memory utilization for calculating correlations will be the same regardless of genomic density. (B) Ground truth whole genome sequencing data is encoded as binary values representing the presence (1) or absence (0) of the reference allele (blue) and alternative allele (red). (C) Variant masking (setting both alleles as absent, represented by 0, corrupts data inputs at a gradually increasing masking rate). Example masked variants are outlined. (D) Fully-connected autoencoders spanning segments can be defined as shown in panel (A), can then be trained to reconstruct the original uncorrupted data from corrupted inputs; (E) the reconstructed outputs (imputed data) can be compared to the ground truth states for loss calculation and are decoded back to genotypes.

FIG. 2 depicts HMM-based (y-axis) versus autoencoder-based (x-axis) imputation accuracy prior to tuning. Minimac4 and untuned autoencoders were tested across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Minimac4 outperformed the untuned autoencoder (left of identity line) and the number of genomic segments in which the untuned autoencoder surpassed Minimac4 (below the identity line). Statistical significance was assessed through two-proportion Z-test p-values.

FIG. 3 depicts HMM-based (y-axis) versus autoencoder-based (axis) imputation accuracy after tuning. Minimac4 and tuned autoencoders were validated across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Minimac4 outperformed the untuned autoencoder (left of identity line) and the number of genomic segments in which the untuned autoencoder surpassed Minimac4 (below the identity line). Statistical significance was assessed through two-proportion Z-test p-values.

FIG. 4 depicts HMM-based versus autoencoder-based imputation accuracy across MAF bins. Autoencoder-based (red) and HMM-based (Minimac4 (blue), Beagle5 (green), and Impute5 (purple)) imputation accuracy was validated across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 5 depicts HMM-based versus autoencoder-based imputation accuracy across ancestry groups. Autoencoder-based (red) and HMM-based (Minimac4 (blue), Beagle5 (green), and Impute5 (purple)) imputation accuracy was validated across individuals of diverse ancestry from MESA cohort (EUR: European (top); EAS: East Asian (2nd row); AMR: Native American (3rd row); AFR: African (bottom)) and multiple genotype array platforms (Affymetrix 6.0 (left), UKB Axiom (middle), Omni1.5M (right)). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 6 depicts HMM-based versus autoencoder-based inference runtimes. Average time and standard error of three imputation replicates were plotted. Two hardware configurations were used for the tests: (A) a low-end environment: 16-core Intel Xeon CPU (E5-2640 v2 2.00 GHZ), 250 GB RAM, and one GPU NVIDIA GTX 1080); (B) a high-end environment: 24-Core AMD CPU (EPYC 7352 2.3 GHZ), 250 GB RAM, using one NVIDIA A100 GPU.

FIG. 7 depicts Beagle5 (y-axis) versus autoencoder-based (x-axis) imputation accuracy prior to tuning. Beagle5 and untuned autoencoders were tested across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Beagle5 outperformed the untuned autoencoder (left of identity line) and the number of genomic segments in which the untuned autoencoder surpassed Beagle5 (below the identity line). Statistical significance was assessed through two-proportion Z-test p-values.

FIG. 8 depicts Impute5 (y-axis) versus autoencoder-based (x-axis) imputation accuracy prior to tuning. Impute5 and untuned autoencoders were tested across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Impute5 outperformed the untuned autoencoder (left of identity line) and the number of genomic segments in which the untuned autoencoder surpassed Impute5 (below the identity line). Statistical significance was assessed through two-proportion Z-test p-values.

FIG. 9 depicts the relationship between genomic segment features and autoencoder performance. Spearman correlations (p) between genomic segment features and autoencoder performance metrics are presented. An “X” denotes Spearman correlations that are not statistically significant (p>0.05). The performance metrics include the mean validation accuracy of Minimac4 and autoencoder (R2_AE_MINUS_MINIMAC), the autoencoder's improvement in accuracy observed after offspring formation (AE_IMPROVEMENT_SIM) and the autoencoder's improvement in accuracy after fine tuning of hyperparameters (AE_IMPROVEMENT_TUNING). The genomic features include the total number of variants per genomic segment in HRC (NVAR_HRC), proportion of rare variants at MAF≤0.5% threshold (RARE_VAR_PROP), proportion of common variants at MAF >0.5% threshold (COMMON_VAR_PROP), number of components needed to explain at least 90% of variance after running Principal Component Analysis (NCOMP), proportion of heterozygous genotypes (PROP_HET), proportion of unique haplotypes (PROP_UNIQUE_HAP) and diplotypes (PROP_UNIQUE_DIP), sum of ratios of explained variance from first two (EXP_RATIO_C1_C2) and three (EXP_RATIO_C1_C2_C3) components from Principal Component Analysis, recombination per variant per variant (REC_PER_SITE), mean pairwise correlation across all variants in each genomic segment (MEAN_LD), mean MAF (MEAN_MAF), GC content of reference alleles (GC_CONT_REF), GC content of alternate alleles (GC_CONT_ALT).

FIG. 10 depicts projecting autoencoder performance from hyperparameters and genomic features. An ensemble-based machine learning approach (Extreme Gradient Boosting-XGBoost) was developed to predict the expected performance (r-squared) of each hyperparameter combination per genomic segment using the results of the coarse-grid search and predictive features calculated for each genomic segment. The observed accuracy of trained autoencoders was plotted versus the accuracy predicted by the XGBoost model after 10-fold cross-validation. Each subplot shows one iteration of the 10-fold validation process and its respective Pearson correlation between the predicted and observed accuracy values in the ARIC validation dataset.

FIG. 11 depicts Beagle5 (y-axis) versus autoencoder-based (axis) imputation accuracy after tuning. Beagle5 and tuned autoencoders were validated across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Beagle5 outperformed the untuned autoencoder (left of identity line) and the number of genomic segments in which the untuned autoencoder surpassed Beagle5 (below the identity line). Statistical significance was assessed through two-proportion Z-test p-values.

FIG. 12 depicts Impute5 (y-axis) versus autoencoder-based (axis) imputation accuracy after tuning. Impute5 and tuned autoencoders were validated across three independent datasets—MESA (top), Wellderly (middle), and HGDP (bottom) and across three genotyping array platforms—Affymetrix 6.0 (left), UKB Axiom (middle), Omni1.5M (right). Each data point represents the imputation accuracy (average r-squared per variant) for an individual genomic segment relative to its WGS-based ground truth. The numerical values presented on the left side and below the identity line (dashed line) indicate the number of genomic segments in which Impute5 outperformed the untuned autoencoder (left of identity line) and the number of genomic segments in which the untuned autoencoder surpassed Impute5 (below the identity line). Statistical significance was assessed through two-proportion Z-test p-values.

FIG. 13 depicts imputation accuracy as a function of unique haplotype abundance. Minimac4 and tuned and untuned autoencoders (AE) were tested across three independent datasets—MESA (top), Wellderly (middle), and HGDP (bottom) and across three genotyping array platforms—Affymetrix 6.0 (left), UKB Axiom (middle), Omni1.5M (right). ‘Many’ vs ‘Few’ haplotypes are defined by splitting genomic segments into those with greater than vs less than the median number of unique haplotypes per genomic segment. Wilcoxon rank-sum tests were applied to compare the untuned and tuned autoencoder to Minimac4. The validation datasets consist of: (A) MESA Affymetrix 6.0; (B) MESA UKB Axiom; (C) MESA Omni 1.5 M; (D) Wellderly Affymetrix 6.0; (E) Wellderly UKB Axiom; (F) Wellderly Omni 1.5 M; (G) HGDP Affymetrix 6.0; (H) HGDP UKB Axiom; (I) HGDP Omni 1.5 M.

FIG. 14 depicts imputation accuracy as a function of unique diplotype abundance. Minimac4 and tuned and untuned autoencoders (AE) were tested across three independent datasets—MESA (top), Wellderly (middle), and HGDP (bottom)—and across three genotyping array platforms—Affymetrix 6.0 (left), UKB Axiom (middle), Omni1.5M (right). ‘Many’ vs ‘Few’ diplotypes are defined by splitting genomic segments into those with greater than vs less than the median number of unique diplotypes per genomic segment. Wilcoxon rank-sum tests were applied to compare the untuned and tuned autoencoder to Minimac4. The validation datasets consist of: (A) MESA Affymetrix 6.0; (B) MESA UKB Axiom; (C) MESA Omni 1.5 M; (D) Wellderly Affymetrix 6.0; (E) Wellderly UKB Axiom; (F) Wellderly Omni 1.5 M; (G) HGDP Affymetrix 6.0; (H) HGDP UKB Axiom; (I) HGDP Omni 1.5 M.

FIG. 15 depicts imputation accuracy as a function of linkage disequilibrium (LD). Minimac4 and tuned and untuned autoencoders (AE) were tested across three independent datasets—MESA (top), Wellderly (middle), and HGDP (bottom)—and across three genotyping array platforms—Affymetrix 6.0 (left), UKB Axiom (middle), Omni1.5M (right). ‘High’ vs ‘Low’ LD is defined by splitting genomic segments into those with greater than vs less than the average pairwise LD strength per genomic segment. Wilcoxon rank-sum tests were applied to compare the untuned and tuned autoencoder to Minimac4. The validation datasets consist of: (A) MESA Affymetrix 6.0; (B) MESA UKB Axiom; (C) MESA Omni 1.5 M; (D) Wellderly Affymetrix 6.0; (E) Wellderly UKB Axiom; (F) Wellderly Omni 1.5 M; (G) HGDP Affymetrix 6.0; (H) HGDP UKB Axiom; (I) HGDP Omni 1.5 M.

FIG. 16 depicts imputation accuracy as a function of data complexity. Minimac4 and tuned and untuned autoencoders (AE) were tested across three independent datasets—MESA (top), Wellderly (middle), and HGDP (bottom)—and across three genotyping array platforms-Affymetrix 6.0 (left), UKB Axiom (middle), Omni1.5M (right). ‘High’ vs ‘Low’ data complexity is defined by splitting genomic segments into those with greater than vs less than the median proportion of variance explained by first two components of Principal Component Analysis per genomic segment (PCA C1+C2). Wilcoxon rank-sum tests were applied to compare the untuned and tuned autoencoder to Minimac4. The validation datasets consist of: (A) MESA Affymetrix 6.0; (B) MESA UKB Axiom; (C) MESA Omni 1.5 M; (D) Wellderly Affymetrix 6.0; (E) Wellderly UKB Axiom; (F) Wellderly Omni 1.5 M; (G) HGDP Affymetrix 6.0; (H) HGDP UKB Axiom; (I) HGDP Omni 1.5 M.

FIG. 17 depicts imputation accuracy as a function of recombination rate. Minimac4 and tuned and untuned autoencoders (AE) were tested across three independent datasets—MESA (top), Wellderly (middle), and HGDP (bottom)—and across three genotyping array platforms-Affymetrix 6.0 (left), UKB Axiom (middle), Omni1.5M (right). ‘High’ vs ‘Low’ recombination rate is defined by splitting genomic segments in those with greater than vs less than the median recombination rate per variant per genomic segment. Wilcoxon rank-sum tests were applied to compare the untuned and tuned autoencoder to Minimac4. The validation datasets consist of: (A) MESA Affymetrix 6.0; (B) MESA UKB Axiom; (C) MESA Omni 1.5 M; (D) Wellderly Affymetrix 6.0; (E) Wellderly UKB Axiom; (F) Wellderly Omni 1.5 M; (G) HGDP Affymetrix 6.0; (H) HGDP UKB Axiom; (I) HGDP Omni 1.5 M.

FIG. 18 depicts HMM-based versus autoencoder-based imputation accuracy across MAF bins (F1 score). Autoencoder-based (red) and HMM-based (Minimac4 (blue), Beagle5 (green), and Impute5 (purple)) imputation accuracy was validated across three independent datasets—MESA (top), Wellderly (middle), and HGDP (bottom)—and across three genotyping array platforms-Affymetrix 6.0 (left), UKB Axiom (middle), Omni1.5M (right). Each data point represents the imputation accuracy (mean F1-score per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values. Please note that F1 scores are high for rare variations given the high degree of class imbalance, most alternative alleles are not present for rare variants, leading to high accuracy in the negative class. R-squared depicted in FIG. 4 provides a more accurate picture of balanced class accuracy.

FIG. 19 depicts HMM-based versus autoencoder-based imputation accuracy across MAF bins concordance. Autoencoder-based (red) and HMM-based (Minimac4 (blue), Beagle5 (green), and Impute5 (purple)) imputation accuracy was validated across three independent datasets—MESA (top), Wellderly (middle), and HGDP (bottom)—and across three genotyping array platforms—Affymetrix 6.0 (left), UKB Axiom (middle), Omni1.5M (right). Each data point represents the imputation accuracy (mean concordance per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values. Please note that F1 scores are high for rare variations given the high degree of class imbalance, most alternative alleles are not present for rare variants, leading to high accuracy in the negative class. R-squared depicted in FIG. 4 provides a more accurate picture of balanced class accuracy.

FIG. 20 depicts TOPMed cohort HMM-based imputation versus HRC cohort autoencoder-based imputation accuracy across MAF bins. Autoencoder-based imputation using the HRC reference panel (red) was compared to HMM-based (Minimac4 (blue), Beagle5 (green), and Impute5 (purple)) imputation accuracy using the upgraded TOPMed cohort. Accuracy was determined across three datasets—MESA (top—not independent), Wellderly (middle-independent), and HGDP (bottom-independent) and across three genotyping array platforms—Affymetrix 6.0 (left), UKB Axiom (middle), Omni1.5M (right). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 21 depicts HMM-based versus autoencoder-based imputation accuracy across ancestry groups. Autoencoder-based (red) and HMM-based (Minimac4 (blue), Beagle5 (green), and Impute5 (purple)) imputation accuracy was validated across individuals of diverse ancestry from HGDP cohort (EUR: European (top); EAS: East Asian (2nd row); AMR: Native American (3rd row); AFR: African (bottom)) and multiple genotype array platforms (Affymetrix 6.0 (left), UKB Axiom (middle), Omni1.5M (right)). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 22 depicts TOPMed cohort HMM-based versus HRC cohort autoencoder-based imputation accuracy across ancestry groups. Autoencoder-based imputation using the HRC reference panel (red) was compared to HMM-based (Minimac4 (blue), Beagle5 (green), and Impute5 (purple)) imputation using the TOPMed reference panel. Accuracy was determined across individuals of diverse ancestry from the HGDP cohort (EUR: European (top); EAS: East Asian (2nd row); AMR: Native American (3rd row); AFR: African (bottom)) and multiple genotype array platforms (Affymetrix 6.0 (left), UKB Axiom (middle), Omni1.5M (right)). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 23 depicts TOPMed cohort HMM-based versus HRC cohort autoencoder-based imputation accuracy across ancestry groups. Autoencoder-based imputation using the HRC reference panel (red) was compared to HMM-based (Minimac4 (blue), Beagle5 (green), and Impute5 (purple)) imputation using the TOPMed reference panel. Accuracy was determined across individuals of diverse ancestry from the MESA cohort (EUR: European (top); EAS: East Asian (2nd row); AMR: Native American (3rd row); AFR: African (bottom)) and multiple genotype array platforms (Affymetrix 6.0 (left), UKB Axiom (middle), Omni1.5M (right)). Each data point represents the imputation accuracy (average r-squared per variant) relative to WGS-based ground truth across MAF bins. Error bars represent standard errors. Wilcoxon rank-sum tests were applied to compare the HMM-based tools to the tuned autoencoder (AE). * represents p-values≤0.05, ** indicates p-values≤0.001, and *** indicates p-values≤0.0001, ns represents non-significant p-values.

FIG. 24 depicts a standard schematic of a general autoencoder.

FIG. 25 depicts predicted probability (x_t) versus cumulative scaled loss for different γ(g) values. Mean squared error (MSE) is shown for comparative purposes.

FIG. 26 depicts multi-GPU training. (A) In multi-GPU mode, different sets of layers are assigned to different GPUs, thus enabling larger models to be distributed across a set of limited memory GPUs (e.g. V100 with 16 GB VRAM). One to three GPUs are sufficient to fit the range of Imputation Autoencoder models without hitting significant GPU-CPU communication bottlenecks. The number of GPUs is assigned based on the number of variants on each genomic segment (i.e. 1×GPU for <2K SNPs, 2×GPUs for 2K-5.5K SNPs etc.). The core dependency for this multi-GPU setup is Thinc, a lightweight deep learning library with a type-checked, functional-programming API for composing models (https://thinc.ai). (B) Example of multi-GPU models deployed on a OLCF Summit compute node (2 sockets, 42 CPUs, 6 GPUs, 2 memory blocks). Each of the two models is assigned a resource set with 6 CPUs, 3 GPUs and 1 memory block. (C) Overview of the pipeline architecture for best model search. For each genomic segment, the corresponding input data are parsed and serialized through Pydantic (a library for data validation and settings management using Python type annotations, https://github.com/pydantic). Next, hundreds of multi-GPU models (as in A) with different sets of hyperparameters (“trials”) are asynchronously trained in parallel for 500 epochs and validated in order to find the most accurate models. The trials are generated by Optuna, an automatic hyperparameter optimization framework (https://optuna.org). A Redis database https://redis.io) is set up for groups of trials so model training can be eventually resumed. Asynchronous I/O is managed through the Asyncio Python library (https: pypi.org/project/asyncio).

### DETAILED DESCRIPTION

Embodiments of the invention relate to the use of artificial neural networks, such as autoencoders, in population genomics and individual genome processing as a way to fill-in missing data from genomic assays with significant sparsity, like low-pass whole genome sequencing or array-based genotyping¹⁰⁻¹². Autoencoders are neural networks tasked with the problem of simply reconstructing the original input data, with constraints applied to the network architecture or transformations applied to the input data in order to achieve a desired goal like dimensionality reduction or compression, and de-noising or de-masking¹³⁻¹⁵, stochastic noise or masking is used to modify or remove data inputs, training the autoencoder to reconstruct the original uncorrupted data from corrupted inputs¹⁶. These autoencoder characteristics are well-suited for genotype imputation and may address some of the limitations of HMM-based imputation by eliminating the need for dissemination of reference panels and allowing the capture of non-linear relationships in genomic regions with complex linkage disequilibrium structures. Some attempts at genotype imputation using neural networks have been previously reported, though for specific genomic contexts¹⁷ at genotype masking levels (5%-20%) not applicable in typical real-world population genetics scenarios¹⁸⁻²¹, or in contexts where the neural network must be re-trained on different input variant sets²⁰.

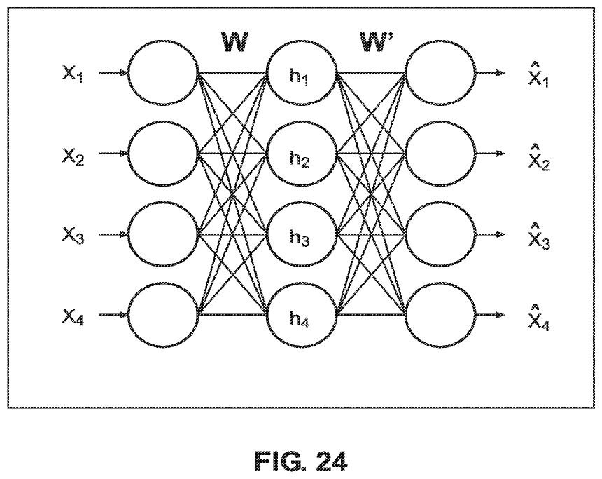

The instant invention relates to an autoencoder-based neural network approach for the simultaneous execution of genetic imputation that can include feature extraction and dimensionality reduction for downstream tasks. An autoencoder is a neural network whose target output (x) is the same as input (x) (FIG. 24). Constraints can be placed on the autoencoder so that it encodes the input data into a non-trivial hidden representation (h) through a weight matrix (W). For genetic data, h is a re-encoding of the data into a set of latent genetic factors. These latent factors have been shown to improve the performance of neural networks for inference in other contexts. Latent genetic factors can similarly improve downstream genetic risk prediction as they will encode the naturally occurring linkage disequilibrium relationships—a factor that has been shown to improve polygenic risk prediction in other contexts. In addition, the unsupervised nature of autoencoders allows one to leverage a large volume of unlabeled genotype data to improve downstream risk predictions.

To accomplish this task, autoencoders can be tiled across the genome. The boundaries of the tiling are determined by a combination of video memory limitations and minima in the linkage disequilibrium patterns of known genetic variants—i.e., minima in the correlation structure of genetic variants associated with regions of high recombination.

### Input and Output Encoding.

All genotypes of each genetic variant can be converted to binary values representing the presence (1) or absence (0) of the reference allele A and alternative allele B, respectively:

$${x}_{i} = \{ \begin{matrix}if ( {G}_{i} = [ A , A ] ) : & {x}_{i} = [ 1 , 0 ] \\ if ( {G}_{i} = [ A , B ] ) : & {x}_{i} = [ 1 , 1 ] \\ if ( {G}_{i} = [ B , A ] ) : & {x}_{i} = [ 1 , 1 ] \\ if ( {G}_{i} = [ B , B ] ) : & {x}_{i} = [ 0 , 1 ] \\ if ( {G}_{i} = [ null ] ) : & {x}_{i} = [ 0 , 0 ]\end{matrix} \}$$

Where G can represent the original genotypes and x can represent the encoded genotype for variant i that is used as input for the autoencoder. The output of the autoencoder can follow the same format: the activation function returns a continuous value for each allele that can be rescaled to these values, for example the tanh function returns a continuous value for each allele that can range between-2 and 2, which is then rescaled to 0-1 range, where 1 represents allele presence and 0 represents allele absence. The scaled outputs can also be regarded as probabilities and can be combined for the calculation of alternative allele dosage, and genotype probabilities. This binary presentation extends one-hot-encoding from the genotype level to the allele level to take into account interdependencies among classes. For example, [A,A] class shares one allele with [A,B], so the binary representation makes these classes share one common value ([1,0] and [1,1], respectively), whereas one-hot-encoding at the genotype-level would mistakenly regard these values as totally independent classes, and causing unnecessary additional allocation of memory ([1,0,0] as [A,A], [0,1,0] as [A,B] or [B,A], and [0,0,1] as [B,B]). Missing genotypes can be represented as absence or 0 probability for all alleles ([0,0]), similar to the one-hot encoding representation of missing values (e.g. [0,0,0]).

### Autoencoder Models.

The autoencoder can be trained using randomly masked whole-genome sequence data from the Haplotype Reference Consortium or any other large collection of genetic data. This autoencoder can take raw genotype data as input and performs imputation as part of its autoencoding function. Random masking of input genetic data can be performed at a low masking rate, which is incremented in subsequent training rounds until a maximum masking rate is achieved. The training performance and autoencoder complexity can determine the maximum masking rate, which can reach up to Ni-5 where N is the total number of input variants of autoencoder i. In order for the autoencoder to provide interesting latent representations that are useful for feature extraction and dimensionality reduction, constraints can be placed on the autoencoder. In one embodiment, sparsity (both node activation sparsity and edge weight regularization) and de-masking constraints can be used to find non-trivial latent genetic factors (h). Many activation functions can be used. In some embodiments, they system uses the commonly used tanh function though reasonable accuracy can be achieved with leaky rectified linear units and sigmoid activation functions. Many loss functions can be used. In some embodiments, the system can use a modified version of cross entropy as the loss function, which can be customized for improving performance when training on highly sparse data with extreme class unbalance, a typical characteristic of genetic variants. Dimensionality reduction can be achieved by using a hidden layer that has a smaller number of nodes than the input/output layers, or by training an autoencoder with the same or larger number of hidden nodes than input/output layers with forced sparsity and trimming of dead nodes that are not used or activated infrequently.

### Loss Function.

The loss function is a measure of the dissimilarity between the predicted (x) and ground truth (x) genotype values. In other words, it is a measure of how accurately the hidden representation (h) can be decoded to reconstruct the original genotype data. The goal when training an autoencoder is to minimize the value of the loss function. In some embodiments, the system uses Sparse Focal Loss (SFL):

$$SFL = {{α}_{t} ( 1 - {x}_{t} )}^{γ} ( CE ) + β * {S}_{( ρ ‖ \hat{ρ} )}$$

Where the class imbalance weighting factor at is represented as the inverse form of class frequency (F) of a genetic variant j:

$${α}_{t} = \{ \begin{matrix}if ( {x}_{i} = 1 ) : & 1 / F \\ if ( {x}_{i} = 0 ) : & 1 / ( 1 - F )\end{matrix} \} \\ {F}_{j} = min ( {p}_{j} , {q}_{j} ) \\ {p}_{j} = f ( {[ 1 ]}_{j} ) \\ {q}_{j} = f ( {[ 0 ]}_{j} )$$

Where f( ) can represents relative frequencies of each allele across genotypes observed for genetic variant j. CE can represent the classic cross entropy loss or log loss of xₜ, which is the predicted class probability:

$$CE = - log ( {x}_{t} ) \\ where : \\ {x}_{t} = \{ \begin{matrix}if ( {x}_{i} = 1 ) : & {\hat{x}}_{i} \\ if ( {x}_{i} = 0 ) : & ( 1 - {\hat{x}}_{i} )\end{matrix} \}$$

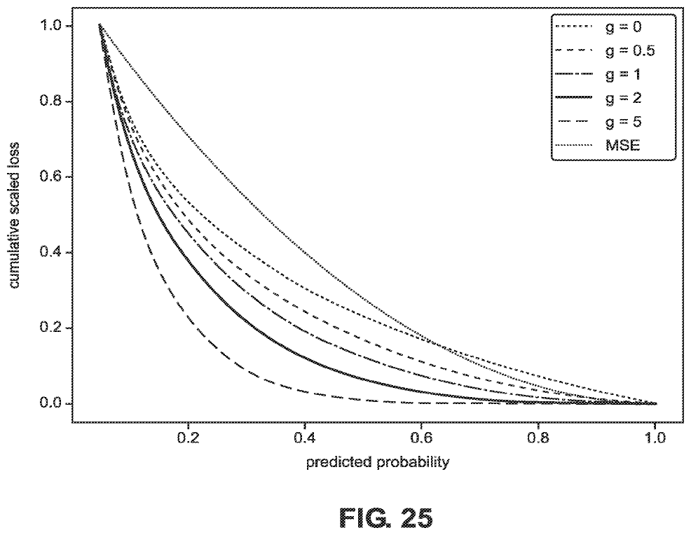

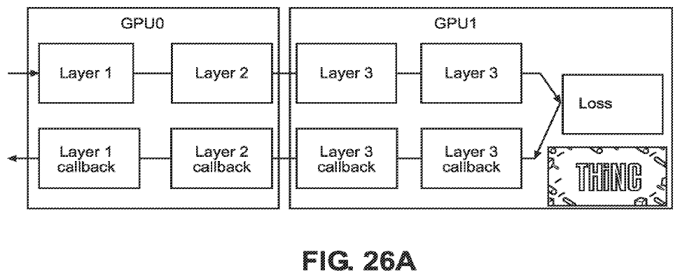

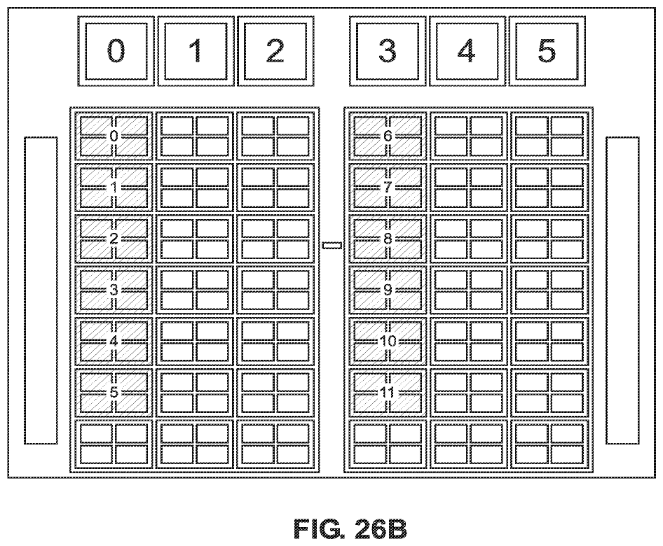

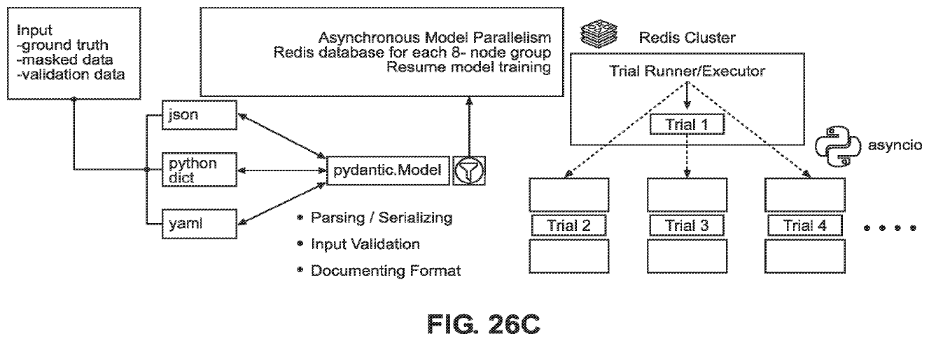

The focal loss element (1−xₜ)^{γ} amplifies the effect of the weights as the class probability values become more imbalanced (FIG. 25), helping the autoencoder to converge into better solutions in fewer learning steps. The sparsity term β*S_{(ρ∥\hat{ρ})} is described in next section.

### De-Noising and Sparsity.

Both the de-noising and sparse autoencoder subtypes can have useful features for the generation of latent genetic factors. De-noising autoencoders add noise to the input (x*), but calculate the loss by comparing the output (\hat{x}) to the original input (x). In some embodiments, de-masking (setting genotype values to missing rather than random values) can be used at an escalating corruption rate (c) to force the autoencoder to learn the underlying linkage disequilibrium structure, perform imputation, and create accurate reconstructions from noisy genotype data. De-noising can also be useful for the correction of genotyping errors, much in the same way imputation could be used to detect potential variant calling errors. Sparse autoencoders can accomplish the goal of dimensionality reduction by explicitly forcing sparsity in the hidden representation (h) through the addition of a sparsity penalty(S) to the loss function:

$${S}_{( ρ ‖ \hat{ρ} )} = {\sum }_{i = 1}^{n} ρ * log ( \frac{ρ}{{\hat{ρ}}_{i}} ) + ( 1 - ρ ) * log ( \frac{1 - ρ}{1 - {\hat{ρ}}_{i}} )$$

where S can be the sparsity loss between the sparsity parameter (\hat{ρ}) that represents the target probability that a hidden node in the hidden layer will get activated, and \hat{ρ} that represents the observed mean activation over a training batch i. n represents the total number of batches. In addition, a regularization term (L1 and/or L2) can be introduced to both autoencoder subtypes to prevent overfitting—which can introduce sparsity to the weight matrix (W):

$$L 1 = \frac{λ}{2} {\sum }_{t = 1}^{T} {\sum }_{h = 1}^{n} ( {W}_{t , h} ) \\ L 2 = \frac{λ}{2} {\sum }_{t = 1}^{T} {\sum }_{h = 1}^{n} {( {W}_{t , h} )}^{2}$$

where L1 and L2 penalties can encourage weights equal to zero in the weight matrix (W) the contribution of which is set by weight decay hyperparameter λ, this calculation can be performed for each hidden node (h=1 through n hidden nodes). L1 can penalize the absolute values of weights, which can reduce weights to zero and therefore compress the model, whereas L2 penalizes their squared form, reducing the weights to values near zero. The final Sparse Focal Loss function, including L1 and L2 regularizers, can be represented as:

$$SFL = {{α}_{t} ( 1 - {x}_{t} )}^{γ} ( CE ) + β * {S}_{( ρ ‖ \hat{ρ} )} + L 1 + L 2$$

### Autoencoder Evaluation and Selection.

Many combinations of hyperparameters can produce high accuracy genetic imputation. In some embodiments, a random grid search approach can be used to evaluate the reconstruction quality of autoencoders using a range of model parameters-corruption parameters (c), regularization parameters (2 for L1 vs L2), and sparsity parameters (ρ and β), and across all model frameworks—the combinations of hidden node activation functions (rectified linear unit, tanh, sigmoid), by loss functions (squared-error, or multinomial logistic loss). 10-fold cross validation can be used to combat overfitting. Parameter tuning can be performed with a greedy genetic search algorithm starting from the top 10 parameter sets from the grid search. Final models for each region can be selected based on best performance on an independent set of 1,000 Genomes individuals with both genotype and WGS data available.

Sample-level reconstruction quality can be quantified using a number of similarity metrics including identity-by-state, concordance, imputation quality score (IQS), r-squared, and F-score. The amount of training required can be guided by these accuracy measures-training continues until these accuracy measures level off per region of the genome.

Embodiments of the invention relate to a generalized approach to unphased human genotype imputation using sparse, denoising autoencoders capable of highly accurate genotype imputation at genotype masking levels (98+%) appropriate for array-based genotyping and low-pass sequencing-based population genetics initiatives. The Examples describe the initial training and implementation of autoencoders spanning all of human chromosome 22, achieving equivalent to superior accuracy relative to modern HMM-based methods, and dramatically improving computational efficiency at deployment without the need to distribute reference panels.

The unphased human genotype imputation approach can be extended to phased human genotype imputation by pre-phasing input genotypes using available algorithms and modifying the encoding to reflect allele presence at each phased allele, e.g.:
if(G_i==[A,A]): x_i=([1,0], [1,0])
if(G_i==[A,B]): x_i=([1,0], [0,1])
if(G_i==[B,A]): x_i=([0,1], [1,0])
if(G_i==[B,B]): x_i=([0,1], [0,1])
if(G_i==[null]): x_i=([0,0], [0,0])

In some embodiments of the invention, the approach can similarly be extended to different organisms with different levels of ploidy by extending the encoding further.

Further information can be found in the reference: Raquel Dias, Doug Evans, Shang-Fu Chen, Kai-Yu Chen, Salvatore Loguercio, Leslie Chan, Ali Torkamani (2022) Rapid, Reference-Free human genotype imputation with denoising autoencoders eLife 11: e75600, which is hereby incorporated by reference in its entirety herein.

### EXAMPLES

### Example 1

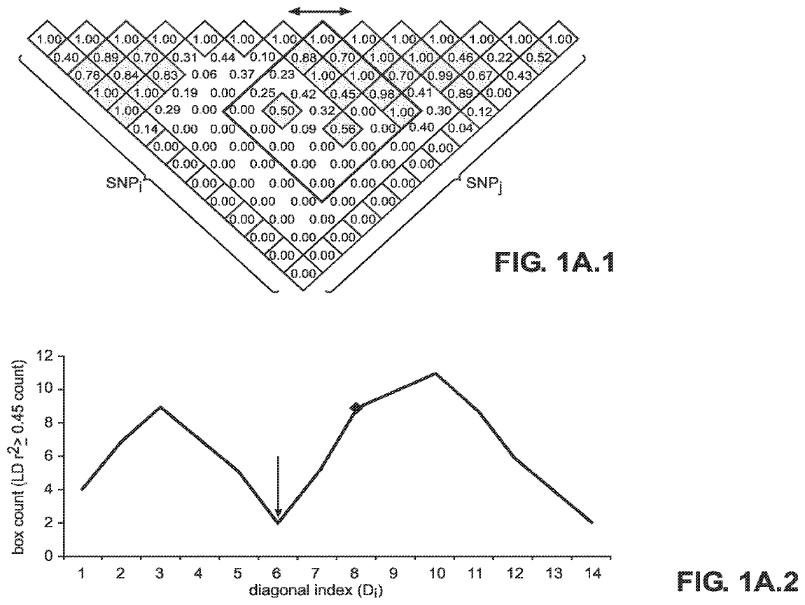

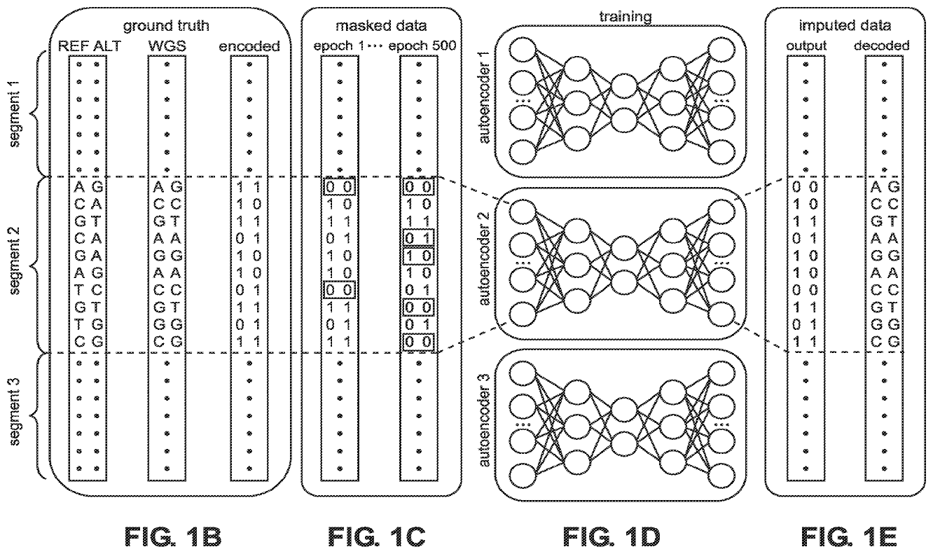

Sparse, de-noising autoencoders spanning all bi-allelic SNPs observed in the Haplotype Reference Consortium were developed and optimized. Each bi-allelic SNP was encoded as two binary input nodes, representing the presence or absence of each allele (FIG. 1B, E). This encoding allows for the straightforward extension to multi-allelic architectures and non-binary allele presence probabilities. A data augmentation approach using modeled recombination events and offspring formation coupled with random masking at an escalating rate drove the autoencoder training strategy (FIG. 1C). Because of the extreme skew of the allele frequency distribution for rarely present alleles 22, a focal-loss-based approach was essential to genotype imputation performance. The basic architecture of the template fully-connected autoencoder before optimization to each genomic segment is depicted in FIG. 1D. Individual autoencoders were designed to span genomic segments with boundaries defined by computationally identified recombination hotspots (FIG. 1A). The starting point for model hyperparameters were randomly selected from a grid of possible combinations and were further tuned from a battery of features describing the complexity of the linkage-disequilibrium structure of each genomic segment.

### Genotype Encoding

Genotypes for all bi-allelic SNPs were converted to binary values representing the presence (1) or absence (0) of the reference allele A and alternative allele B, respectively, as shown in Equation 1.

$${x}_{i} = \{ \begin{matrix}if ( {G}_{i} = [ A , A ] ) : & {x}_{i} = [ 1 , 0 ] \\ if ( {G}_{i} = [ A , B ] ) : & {x}_{i} = [ 1 , 1 ] \\ if ( {G}_{i} = [ B , A ] ) : & {x}_{i} = [ 1 , 1 ] \\ if ( {G}_{i} = [ B , B ] ) : & {x}_{i} = [ 0 , 1 ] \\ if ( {G}_{i} = [ null ] ) : & {x}_{i} = [ 0 , 0 ]\end{matrix} \} \qquad ( 1 )$$

Where x is a vector containing the two allele presence input nodes to the autoencoder and their encoded allele presence values derived from the original genotype, G, of variant i. The output nodes of the autoencoder, regardless of activation function, are similarly rescaled to 0-1. The scaled outputs can also be regarded as probabilities and can be combined for the calculation of alternative allele dosage and/or genotype probabilities. This representation maintains the interdependencies among classes, is extensible to other classes of genetic variation, and allows for the use of probabilistic loss functions.

### Training Data, Masking, and Data Augmentation

Training Data. Whole-genome sequence data from the Haplotype Reference Consortium (HRC) was used for training and as the reference panel for comparison to HMM-based imputation²³. The dataset consists of 27,165 samples and 39,235,157 biallelic SNPs generated using whole-genome sequence data from 20 studies of predominantly European ancestry (HRC Release 1.1): 83.92% European, 2.33% East Asian, 1.63% Native American, 2.17% South Asian, 2.96% African, and 6.99% admixed ancestry individuals. Genetic ancestry was determined using continental population classification from the 1000 Genomes Phase3 v5 (1000G) reference panel and a 95% cutoff using Admixture software²⁴. Genotype imputation autoencoders were trained for all 510,442 unique SNPs observed in HRC on human chromosome 22. For additional comparisons, whole-genome sequence data from 31 studies available through the NHLBI Trans-Omics for Precision Medicine (TOPMed) program were used as an alternative reference panel for HMM-based imputation tools (Taliun et al., 2021). Freeze 8 of TOPMed was downloaded, which is the latest version with all consent groups genotyped across the same set of jointly called variants. GRCh38 TOPMed cohorts were converted to hg19 with Picard 2.25 (‘Picard toolkit’, 2019), and multi allelic SNPs removed with bcftools v.1.10.2 (Danecek et al., 2021). Any variants with missing genotypes were excluded as well, yielding a final reference panel for chr22 consisting of 73,586 samples and 11,089,826 biallelic SNPs. Since the ARIC and MESA cohorts are used for model selection and validation, they were excluded from the TOPMed reference panel. Relatedness analysis using the KING robust kinship estimator revealed significant data leakage boosting HMM-based imputation performance through individuals directly participating in the MESA and other TOPMed cohorts, as well as through numerous first- and second-degree familial relationships spanning MESA individuals and individuals in other TOPMed cohorts.

Validation and Testing Data. A balanced (50%: 50% European and African genetic ancestry) subset of 796 whole genome sequences from the Atherosclerosis Risk in Communities cohort (ARIC)²⁵, was used for model validation and selection. The Wellderly²⁶, Human Genome Diversity Panel (HGDP)²⁷, and Multi-Ethnic Study of Atherosclerosis (MESA)²⁸ cohorts were used for model testing. The Wellderly cohort consisted of 961 whole genomes of predominantly European genetic ancestry. HGDP consisted of 929 individuals across multiple ancestries: 11.84% European, 14.64% East Asian, 6.57% Native American, 10.98% African, and 55.97% admixed. MESA consisted of 5,370 whole genomes across multiple ancestries: 27.62% European, 11.25% East Asian, 4.99% Native American, 5.53% African, and 50.61% admixed.

GRCh38 mapped cohorts (HGDP and MESA) were converted to hg19 using Picard v2.25²⁹. All other datasets were originally mapped and called against hg19. Multi-allelic SNPs, SNPS with >10% missingness, and SNPs not observed in HRC were removed with bcftools v1.10.2³⁰. Mock genotype array data was generated from these WGS cohorts by restricting genotypes to those present on commonly used genotyping arrays (Affymetrix 6.0, UKB Axiom, and Omni 1.5M). For chromosome 22, intersection with HRC and this array-like masking respectively resulted in: 9,025, 10,615, and 14,453 out of 306,812 SNPs observed in ARIC; 8,630, 10,325, and 12,969 out of 195,148 SNPs observed in the Wellderly; 10,176, 11,086, and 14,693 out of 341,819 SNPs observed in HGDP; 9,237, 10,428, and 13,677 out of 445,839 SNPs observed in MESA.

Data Augmentation. The inventors employed two strategies for data augmentation-random variant masking and simulating further recombination with offspring formation. During training, random masking of input genotypes was performed at escalating rates, starting with a relatively low masking rate (80% of variants) that is gradually incremented in subsequent training rounds until up to only 5 variants remain unmasked per autoencoder. Masked variants are encoded as the null case in Equation 1. During finetuning the inventors used sim1000G to simulate of offspring formation using the default genetic map and HRC genomes as parents. A total of 30,000 offspring genomes were generated and merged with the original HRC dataset, for a total of 57,165 genomes.

### Loss Function

In order to account for the overwhelming abundance of rare variants, the accuracy of allele presence reconstruction was scored using an adapted version of focal loss (FL) [32], shown in Equation 2.

$$FL = - {{α}_{t} ( 1 - {p}_{t} )}^{γ} [ {x}_{t} log ( {p}_{t} ) + ( 1 - {x}_{t} ) log ( 1 - {p}_{t} ) ] \qquad ( 2 )$$

where the classic cross entropy (shown as binary log loss in brackets) of the truth class (xₜ) predicted probability (pₜ) is weighted by the class imbalance factor αₜ and a modulating factor (1−pₜ)^{γ}. The modulating factor is the standard focal loss factor with hyperparameter, γ, which amplifies the focal loss effect by down-weighting the contributions of well-classified alleles to the overall loss (especially abundant reference alleles for rare variant sites). αₜ is an additional balancing hyperparameter set to the truth class frequency.

This base focal loss function is further penalized and regularized to encourage simple and sparse models in terms of edge-weight and hidden layer activation complexity. These additional penalties result in the final loss function as shown in Equation 3.

$$SFL = - {{α}_{t} ( 1 - {p}_{t} )}^{γ} [ {x}_{t} log ( {p}_{t} ) + ( 1 - {x}_{t} ) log ( 1 - {p}_{t} ) ] + β {S}_{( ρ ‖ \hat{ρ} )} + {λ}_{1} L 1 + {λ}_{2} L 2 \qquad ( 3 )$$

where L1 and L2 are the standard L1 and L2 norms of the autoencoder weight matrix, with their contributions mediated by the hyperparameters λ₁ and λ₂. S is a sparsity penalty, with its contribution mediated by the hyperparameter β, which penalizes deviation from a target hidden node activation set by the hyperparameter (ρ) vs the observed mean activation \hat{ρ} over a training batch j summed over total batches n, as shown in Equation 4:

$${S}_{( ρ ‖ \hat{ρ} )} = {\sum }_{j = 1}^{n} ρ * log ( \frac{ρ}{{\hat{ρ}}_{j}} ) + ( 1 - ρ ) * log ( \frac{1 - ρ}{1 - {\hat{ρ}}_{j}} ) \qquad ( 4 )$$

### Genome Tiling

All model training tasks were distributed across a diversified set of NVIDIA graphical processing units (GPUs) with different video memory limits: 5× Titan Vs (12 GB), 8×A100s (40 GB), 60×V100s (32 GB). Given computational complexity and GPU memory limitations, individual autoencoders were designed to span approximately independent genomic segments with boundaries defined by computationally identified recombination hotspots (FIG. 1).

These segments were defined using an adaptation of the LDetect algorithm [33]. First, the inventors calculated a n×n matrix of pairwise SNP correlations using all common genetic variation (≥5% minor allele frequency) from HRC. Correlation values were thresholded at 0.45. For each SNP, the inventors calculated a boxsum of all pairwise SNP correlations spanning 500 common SNPs upstream and downstream of the index SNP. This moving boxsum quantifies the overall local LD strength centered at each SNP. Local minima in this moving boxsum were used to split the genome into approximately independent genomic segments of two types—large segments of high LD interlaced with short segments of weak LD corresponding to recombination hotspot regions. Individual autoencoders were designed to span the entirety of a single high LD segment plus its adjacent upstream and downstream weak LD regions. Thus, adjacent autoencoders overlap at their weak LD ends. If an independent genomic segment exceeded the threshold number of SNPs amenable to deep learning given GPU memory limitations, internal local minima within the high LD regions were used to split the genomic segments further to a maximum of 6000 SNPs per autoencoder. Any remaining genomic segments still exceeding 6000 SNPs were further split into 6000 SNP segments with large overlaps of 2500 SNPs given the high degree of informative LD split across these regions. This tiling process resulted in 256 genomic segments: 188 independent LD segments, 32 high LD segments resulting from internal local minima splits, and 36 segments further split due to GPU memory limitations.

### Hyperparameter Initialization and Grid Search

The inventors first used a random grid search approach to define initial hyperparameter combinations producing generally accurate genotype imputation results. The hyperparameters and their potential starting values are listed in Table 1. This coarse-grain grid search was performed on all genomic segments of chromosome 22 (256 genomic segments), each tested with 100 randomly selected hyperparameter combinations per genomic segment, with a batch size of 256 samples, training for 500 epochs without any stop criteria, and validating on an independent dataset (ARIC). To evaluate the performance of each hyperparameter combination, the inventors calculated the average coefficient of determination (r-squared) comparing the predicted and observed alternative allele dosages per variant. Concordance and F1-score were also calculated to screen for anomalies but were not ultimately used for model selection.

TABLE 1
Description and values of hyperparameters tested in grid search.

| Hyperparameter description | Tested values (coarse-grid search) |
|---|---|
| λ₁ for L1 regularization | [1e−3, 1e−4, 1e−5, 1e−6, 1e−1, 1e−2, 1e−7, 1e−8] |
| λ₂ for L2 regularization | [1e−3, 1e−4, 1e−5, 1e−6, 1e−1, 1e−2, 1e−7, 1e−8] |
| Sparsity scaling factor (β) | [0, 0.001, 0.01, 0.05, 1, 5, 10] |
| Target average hidden layer | [0.001, 0.004, 0.007, 0.01, 0.04, 0.07, 0.1, 0.4, 0.7, 1.0] |
| activation (ρ) | |
| Activation function type | [‘sigmoid’, ‘tanh’, ‘relu’, ‘softplus’] |
| Learning rate | [0.000001, 0.00001, 0.0001, 0.001, 0.01, 0.1, 1, 10, 100] |
| Amplifying factor for focal loss (γ) | [0, 0.5, 1, 2, 3, 5] |
| Optimizer type | [‘Adam’, ‘RMS Propagation’, ‘Gradient Descent’] |
| Loss type | [‘Binary Cross Entropy’, ‘Custom Focal Loss’] |
| Number of hidden layers | [1, 2, 4, 6, 8] |
| Hidden layer size ratio | [0.2, 0.3, 0.4, 0.5, 0.6, 0.7, 0.8, 0.9, 1] |
| Learning rate decay ratio | [0.0, 0.25, 0.5, 0.75, 0.95, 0.99, 0.999, 0.9999] |

λ₁: scaling factor for Least Absolute Shrinkage and Selection Operator (LASSO or L1) regularization; λ₂: scaling factor for Ridge (L2) regularization; β: scaling factor for sparsity penalty described in Equation 4; ρ: target hidden layer activation described in Equation 4; Activation function type: defines how the output of a hidden neuron will be computed given a set of inputs; Learning rate: step size at each learning iteration while moving toward the minimum of the loss function; γ: amplifying factor for focal loss described in Equation 3; Optimizer type: algorithms utilized to minimize the loss function and update the model weights in backpropagation; Loss type: algorithms utilized to calculate the model error (Equation 2); Number of hidden layers: how many layers of artificial neurons to be implemented between input layer and output layer; Hidden layer size ratio: scaling factor to resize the next hidden layer with reference to the size of Its previous layer; Learning rate decay ratio: scaling factor for updating the learning rate value on every 500 epochs.

### Hyperparameter Tuning

In order to avoid local optimal solutions and reduce the hyperparameter search space, an ensemble-based machine learning approach was developed (Extreme Gradient Boosting-XGBoost) to predict the expected performance (r-squared) of each hyperparameter combination per genomic segment using the results of the coarse-grid search and predictive features calculated for each genomic segment. These features include the number of variants, average recombination rate and average pairwise Pearson correlation across all SNPs, proportion of rare and common variants across multiple minor allele frequency (MAF) bins, number of principal components necessary to explain at least 90% of variance, and the total variance explained by the first 2 principal components. The observed accuracies of the coarse-grid search, numbering 25,600 training inputs, were used to predict the accuracy of 500,000 new hyperparameter combinations selected from Table 1 without training. All categorical predictors (activation function name, optimizer type, loss function type) were one-hot encoded. The model was implemented using XGBoost package v1.4.1 in Python v3.8.3 with 10-fold cross-validation and default settings.

The inventors then ranked all hyperparameter combinations by their predicted performance and selected the top 10 candidates per genomic segment along with the single best initially tested hyperparameter combination per genomic segments for further consideration. All other hyperparameter combinations were discarded. Genomic segments with sub-optimal performance relative to Minimac were subjected to tuning with simulated offspring formation. For tuning, the maximum number of epochs was increased (35,000) with automatic stop criteria: if there is no improvement in average loss value of the current masking/training cycle versus the previous one, the training is interrupted, otherwise training continues until the maximum epoch limit is reached. Each masking/training cycle consisted of 500 epochs. Final hyperparameter selection was based on performance on the validation dataset (ARIC).

This process results in 256 unique autoencoders spanning the genomic segments of chromosome 22. Each genomic segment consists of a different number of input variables (genetic variants), sparsity, and correlation structure. Thus, 256 unique autoencoder models span the entirety of chromosome 22 (e.g.: each autoencoder has different edge weights, number of layers, loss function, as well as regularization and optimization parameters).

### Performance Testing and Comparisons

Performance was compared to Minimac4³⁴, Beagle5⁵, and Impute5⁴ using default parameters. Population level reconstruction accuracy is quantified by measuring r-squared across multiple strata of data: per genomic segment, at whole chromosome level, and stratified across multiple minor allele frequency bins: [0.001-0.005), [0.005-0.01), [0.01-0.05), [0.05-0.1), [0.1-0.2), [0.2-0.3), [0.3-0.4), [0.4-0.5). While r-squared is the primary comparison metric, sample-level and population-level model performance is also evaluated with concordance and the F1-score. Wilcoxon rank-sum testing was used assess the significance of accuracy differences observed. Spearman correlations were used to evaluate the relationships between genomic segment features and observed imputation accuracy differences. Standard errors for per variant imputation accuracy r-squared is equal or less than 0.001 where not specified. Performance is reported only for the independent test datasets (Wellderly, MESA, and HGDP).

The inventors used the MESA cohort for inference runtime comparisons. Runtime was determined using the average and standard error of three imputation replicates. Two hardware configurations were used for the tests: 1) a low-end environment: 16-core Intel Xeon CPU (E5-2640 v2 2.00 GHz), 250 GB RAM, and one GPU (NVIDIA GTX 1080); 2) a high-end environment: 24-Core AMD CPU (EPYC 7352 2.3 GHZ), 250 GB RAM, using one NVIDIA A100 GPU. The inventors report computation time only, input/output (I/O) reading/writing times are excluded as separately optimized functions.

### Data Availability

The data that support the findings of this study are available from dbGAP and European Genome-phenome Archive (EGA), but restrictions apply to the availability of these data, which were used under ethics approval for the current study, and so are not openly available to the public. The computational pipeline for autoencoder training and validation is available at https://github.com/TorkamaniLab/Imputation_Autoencoder/tree/master/autoencoder_tuning_pipeline. The python script for calculating imputation accuracy is available at https://github <dot> com <slash> TorkamaniLab/imputation_accuracy_calculator.

### Results

### Untuned Performance and Model Optimization.

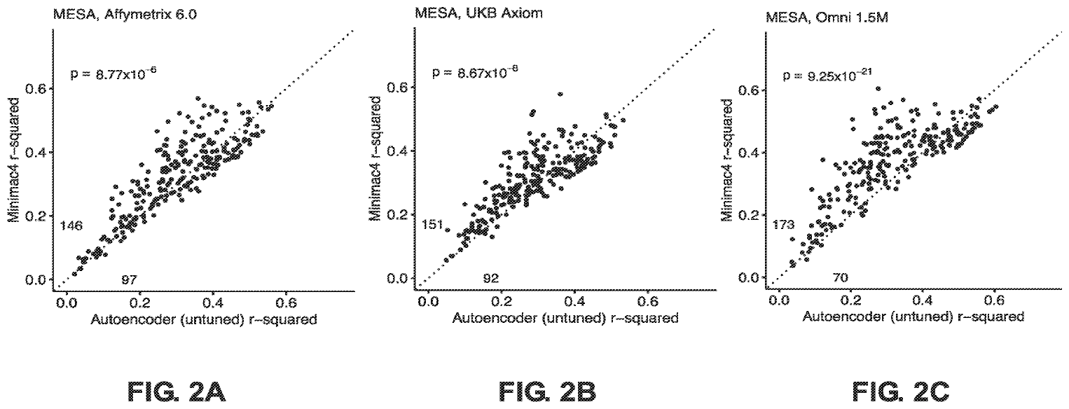

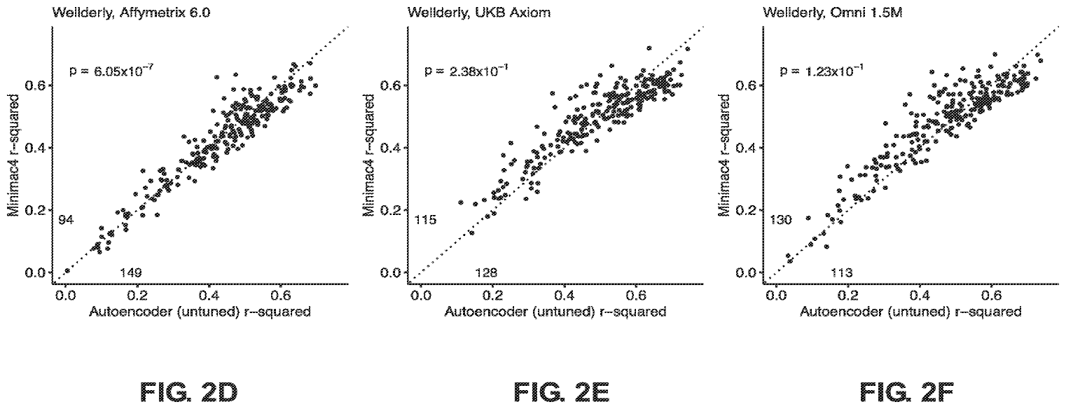

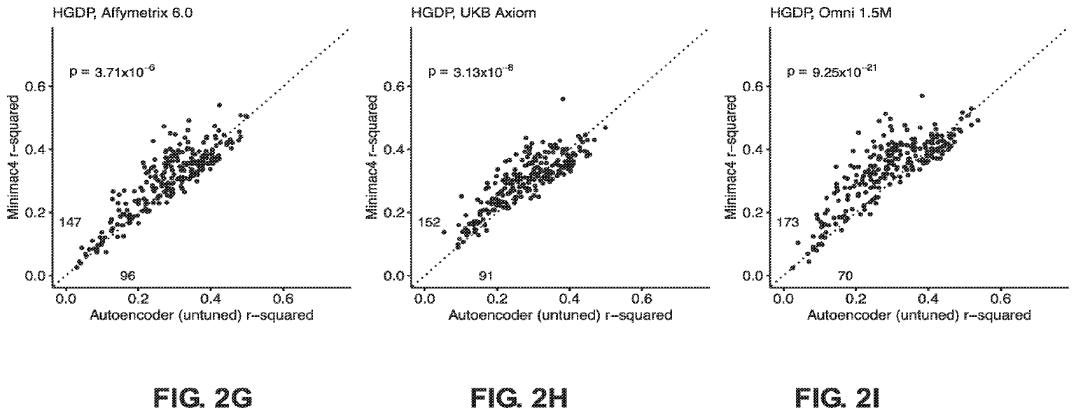

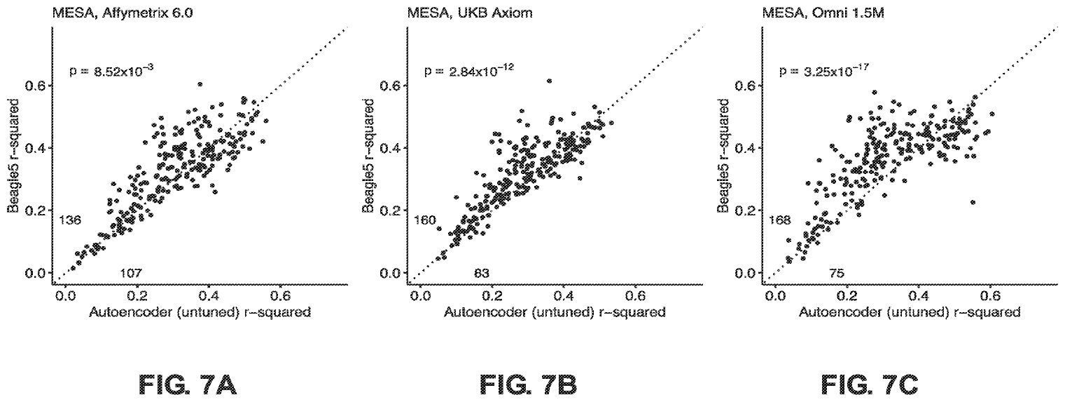

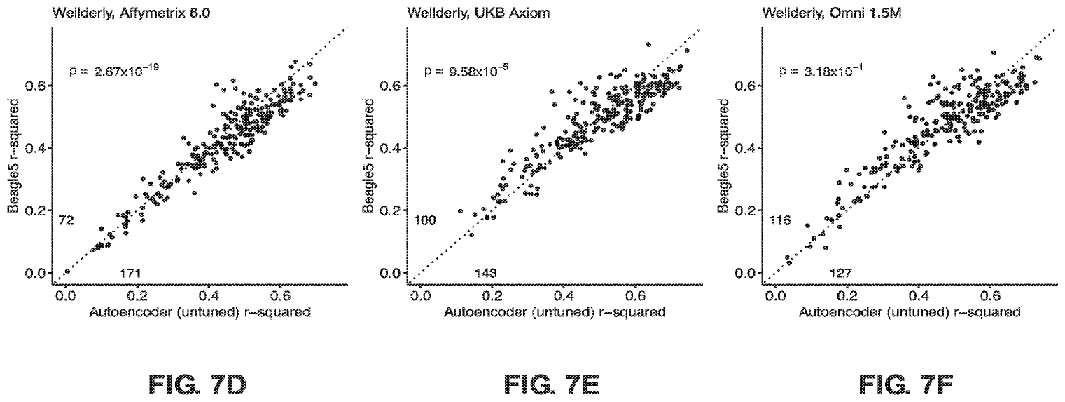

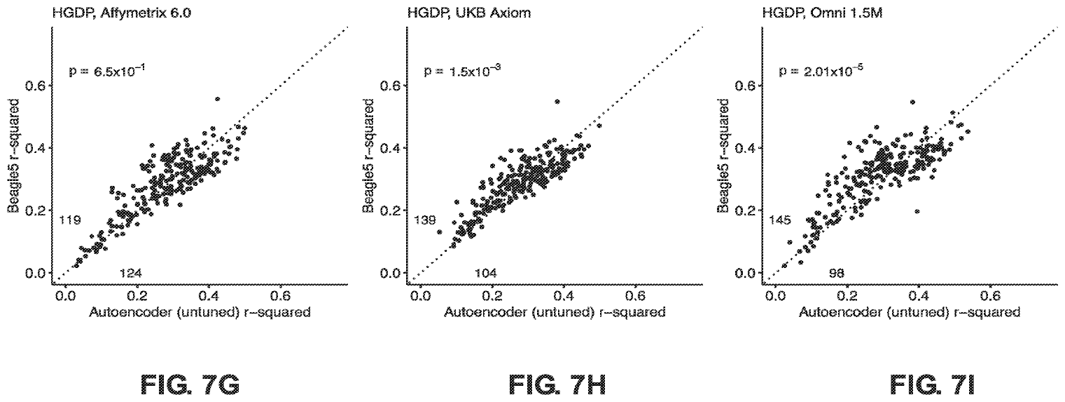

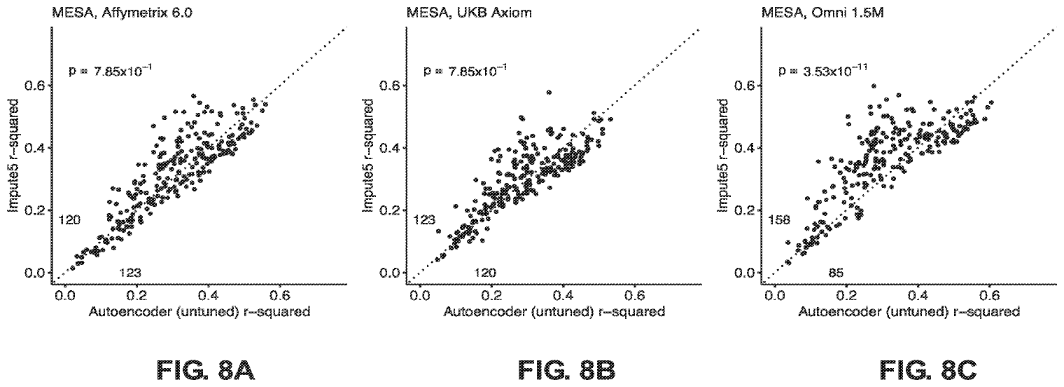

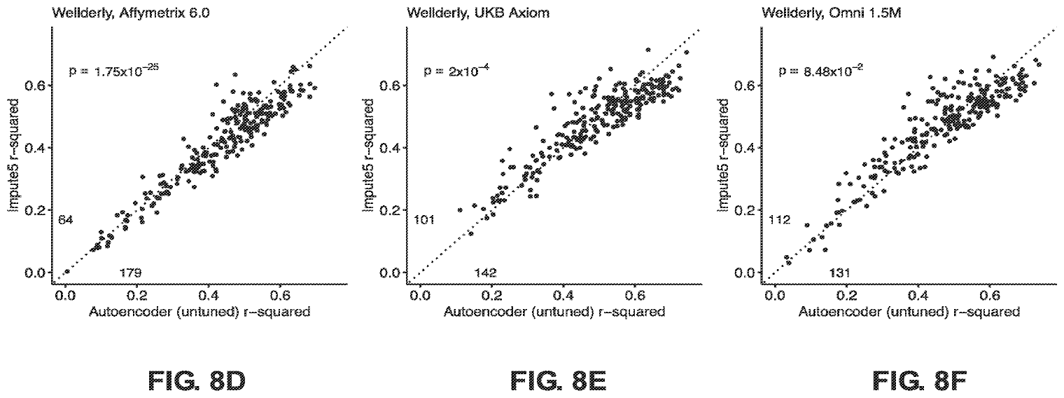

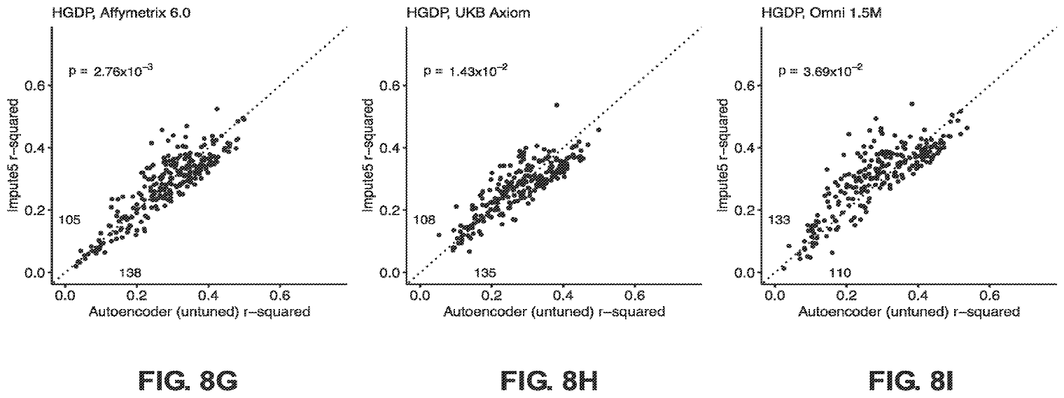

A preliminary comparison of the best performing autoencoder per genomic segment vs HMM-based imputation was made after the initial grid (Minimac4: FIG. 2, Beagle5 and Eagle5: FIG. 7 and FIG. 8). Untuned autoencoder performance was equivalent or inferior to all tested HMM-based methods except when tested on the European ancestry-rich Wellderly dataset when masked using the Affymetrix 6.0 and UKB Axiom marker sets, but not Omni 1.5M markers. HMM-based imputation was consistently superior across the more ancestrally diverse test datasets (MESA and HGDP) (two proportion test, p≤8.77×10⁻⁶). Overall, when performance across genomic segments, test datasets, and test array marker sets was combined, the autoencoders exhibited an average r-squared per variant of 0.352±0.008 in reconstruction of WGS ground truth genotypes versus an average r-squared per variant of 0.374±0.007, 0.364±0.007, and 0.357±0.007 for HMM-based imputation methods (Minimac4, Beagle5, and Impute5, respectively) (Table 2). This difference was statistically significant only relative to Minimac4 (Minimac4: Wilcoxon rank-sum test p=0.037, Beagle5 and Eagle5: p≥0.66).

TABLE 2
Performance comparisons between untuned autoencoder (AE) and
HMM-based imputation tools (Minimac4, Beagle5, and Impute5).

| | | | | Affymetrix | UKB | Omni | |
|---|---|---|---|---|---|---|---|
| | MESA | Wellderly | HGDP | 6.0 | Axiom | 1.5M | Combined |
| AE(untuned) | 0.303 ± | 0.470 ± | 0.285 ± | 0.339 ± | 0.356 ± | 0.362 ± | 0.352 ± |
| | 0.008 | 0.009 | 0.006 | 0.008 | 0.007 | 0.008 | 0.008 |
| Minimac4 | 0.337 ± | 0.471 ± | 0.314 ± | 0.352 ± | 0.370 ± | 0.400 ± | 0.374 ± |
| | 0.007* | 0.008 | 0.006 | 0.008 | 0.006 | 0.007 | 0.007 |
| Beagle5 | 0.336 ± | 0.460 ± | 0.296 ± | 0.342 ± | 0.367 ± | 0.384 ± | 0.364 ± |
| | 0.007 | 0.008 | 0.005 | 0.007 | 0.006 | 0.007 | 0.007 |
| Impute5 | 0.326 ± | 0.458 ± | 0.289 ± | 0.336 ± | 0.354 ± | 0.383 ± | 0.358 ± |
| | 0.007 | 0.008 | 0.006 | 0.008 | 0.006 | 0.008 | 0.007 |

Average r-squared per variant was extracted from each genomic segment of chromosome 22.
Wilcoxon rank-sum tests were applied to compare the HMM-based tools to the reference tuned autoencoder (AE).
*represents p-values ≤ 0.05,
** indicates p-values ≤ 0.001, and
*** indicates p-values ≤ 0.0001.

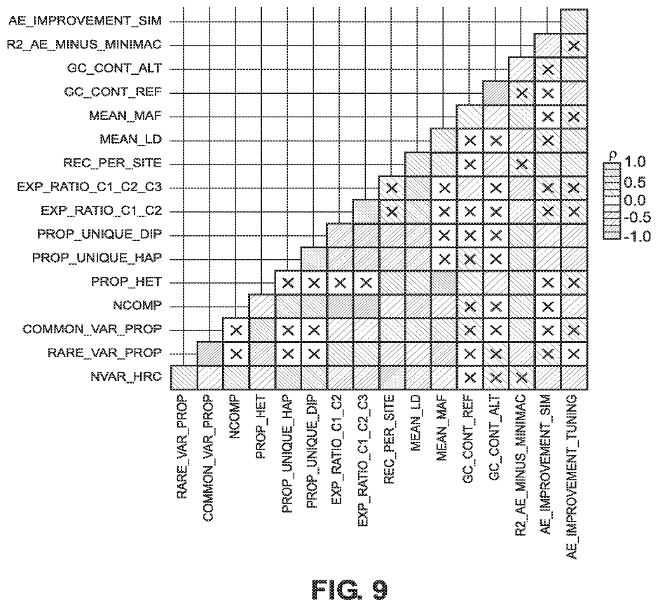

In order to understand the relationship between genomic segment features, hyperparameter values, and imputation performance, the inventors calculated predictive features for each genomic segment and determined their Spearman correlation with the differences in r-squared observed for the autoencoder vs Minimac4 (FIG. 9). The inventors observed that the autoencoder had superior performance when applied to the genomic segments with the most complex LD structures: those with larger numbers of observed unique haplotypes, unique diplotypes, and heterozygosity, as well as high average MAF, and low average pairwise Pearson correlation across all SNPs (average LD) (Spearman correlation (ρ≥0.22, p≤9.8×10⁻⁰⁴). Similarly, the inventors quantified genomic segment complexity by the proportion of variance explained by the first two principal components as well as the number of principal components needed to explain at least 90% of the variance of HRC genotypes from each genomic segment. Concordantly, superior autoencoder performance was associated with a low proportion explained by the first two components and positively correlated with the number of components required to explained 90% of variance (Spearman ρ≥0.22, p≤8.3×10⁻⁰⁴). These observations informed the tuning strategy.

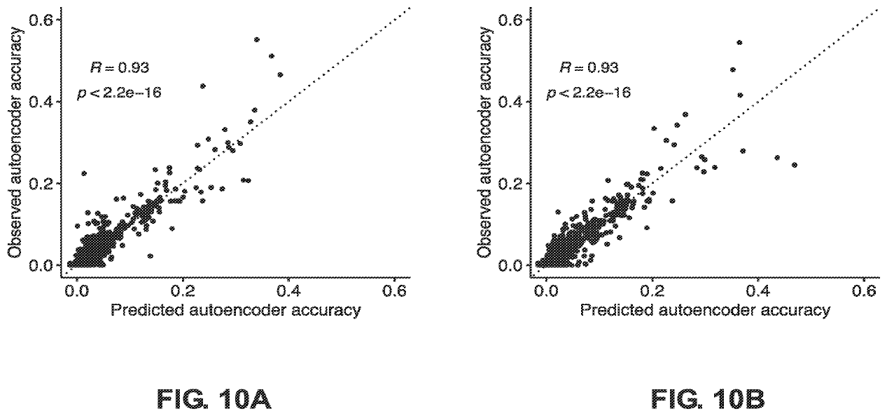

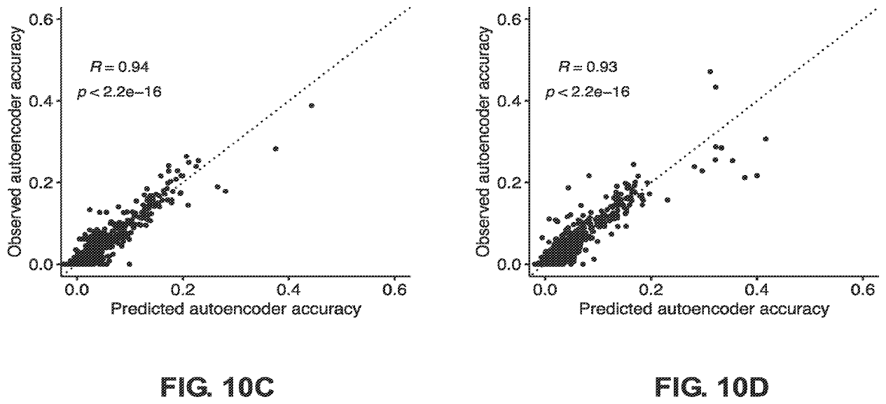

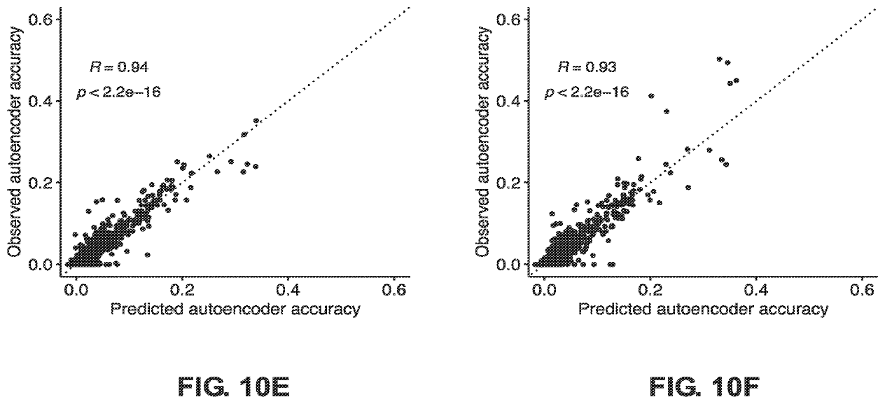

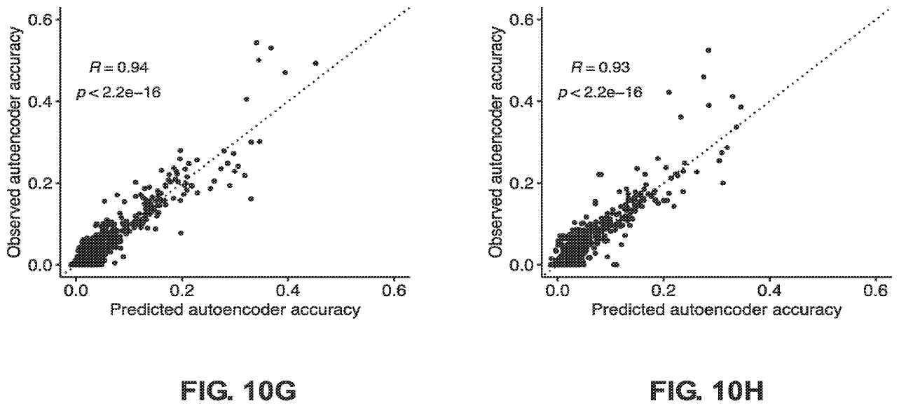

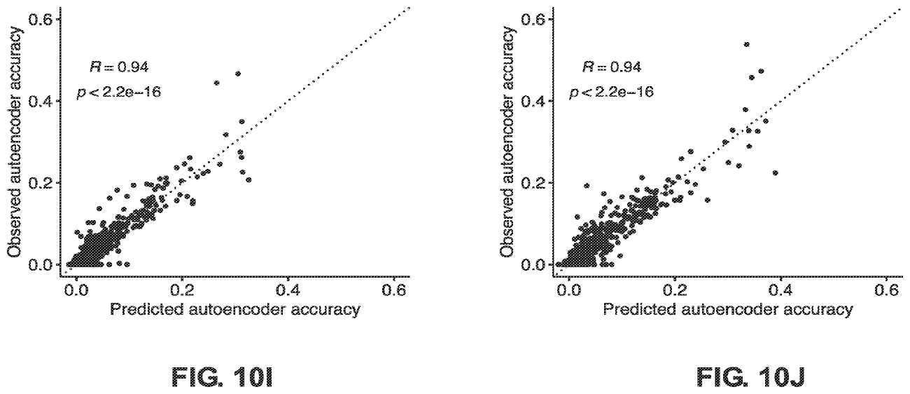

The inventors then used the genomic features significantly correlated with imputation performance to predict the performance of and select the hyperparameter values to advance to fine-tuning. An ensemble model inference approach was able to predict the genomic segment-specific performance of hyperparameter combinations with high accuracy (FIG. 10), mean r-squared=0.935±0.002 of predicted vs observed autoencoders accuracies via 10-fold cross validation). The top 10 best performing hyperparameter combinations were advanced to fine-tuning (Table 3). Autoencoder tuning with simulated offspring formation was then executed as described in herein.

TABLE 3
Top 10 best performing hyperparameter combinations that advanced to fine-tuning.

| | | | | | Learn | | | Loss | Hidden | Size | |
|---|---|---|---|---|---|---|---|---|---|---|---|
| λ₁ | λ₂ | β | ρ | Activation | rate | γ | Optimizer | type | layers | ratio | Decay |
| 0.1 | 0 | 0.01 | 0.01 | tanh | 1.0*10⁻⁴ | 0 | adam | CE | 4 | 1 | 0.95 |
| 0.1 | 0 | 1 | 0.5 | sigmoid | 1.0*10⁻⁴ | 1 | adam | CE | 2 | 0.9 | 0.95 |
| 0.1 | 0 | 5 | 0.5 | sigmoid | 1.0*10⁻¹ | 4 | adam | CE | 2 | 0.5 | 0 |
| 0.1 | 0 | 1 | 0.005 | relu | 1.0*10⁻¹ | 4 | adam | FL | 6 | 1 | 0.25 |
| 0.1 | 0 | 5 | 0.01 | relu | 1.0*10⁻⁵ | 5 | adam | FL | 4 | 1 | 0.95 |
| 0.1 | 0 | 0.01 | 0.1 | leakyrelu | 1.0*10⁻⁵ | 0 | adam | FL | 8 | 0.9 | 0.95 |
| 0.1 | 0 | 1 | 0.01 | tanh | 1.0*10⁻⁴ | 0 | adam | CE | 6 | 1 | 0.95 |
| 0 | 1.0*10⁻⁸ | 0.001 | 0.05 | relu | 1.0*10⁻⁵ | 4 | adam | CE | 8 | 0.6 | 0.95 |
| 0.1 | 0 | 0 | 0.01 | relu | 1.0*10⁻¹ | 5 | adam | FL | 8 | 0.9 | 0 |
| 0.1 | 0 | 0.01 | 0.01 | tanh | 1.0*10⁻³ | 5 | adam | CE | 2 | 1 | 0.95 |

### Tuned Performance.

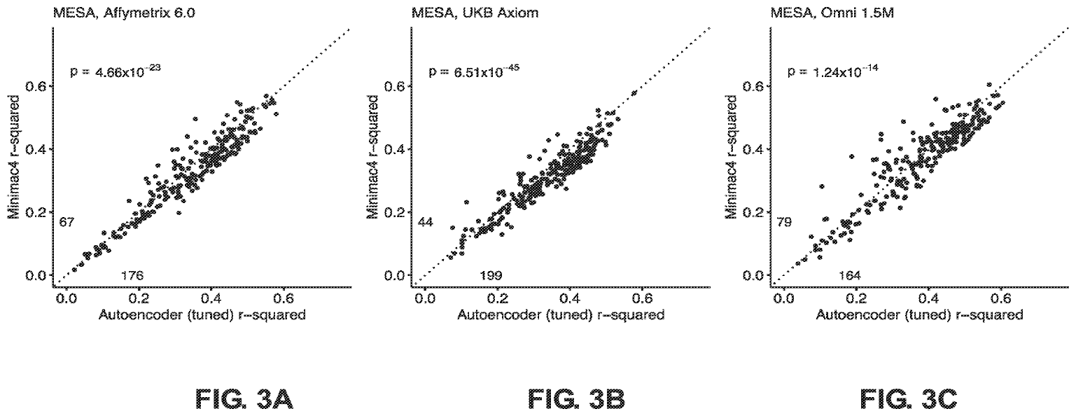

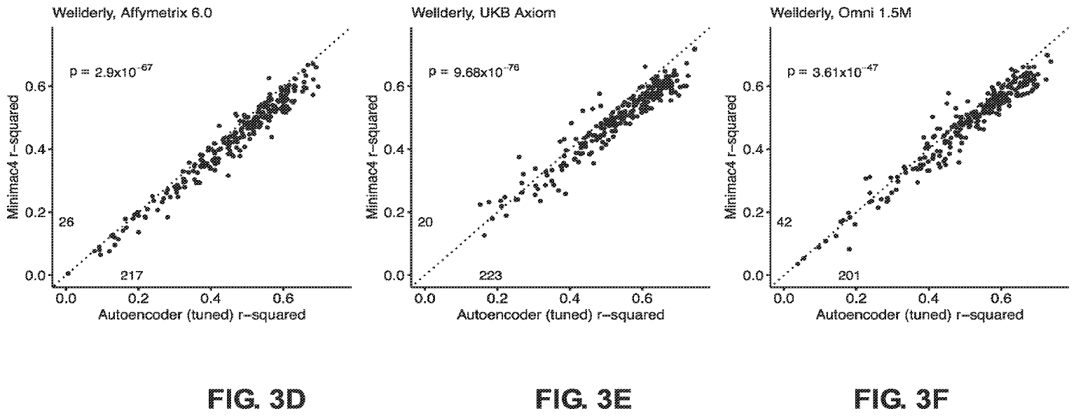

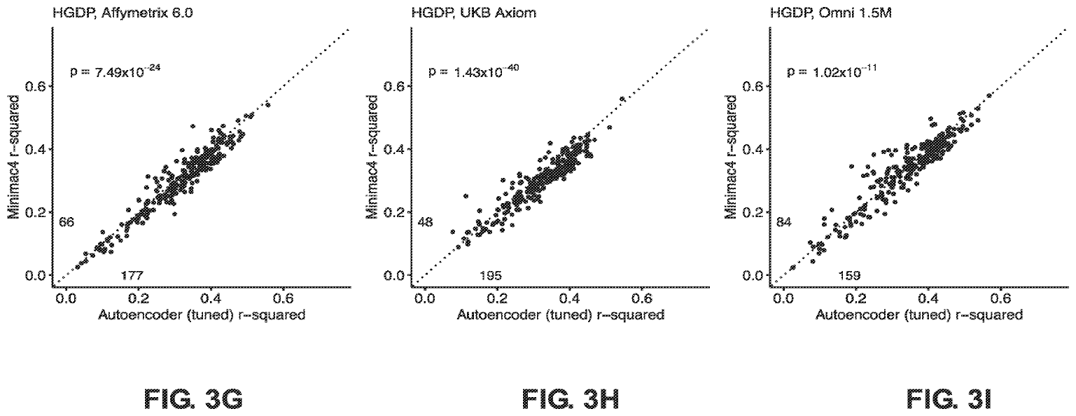

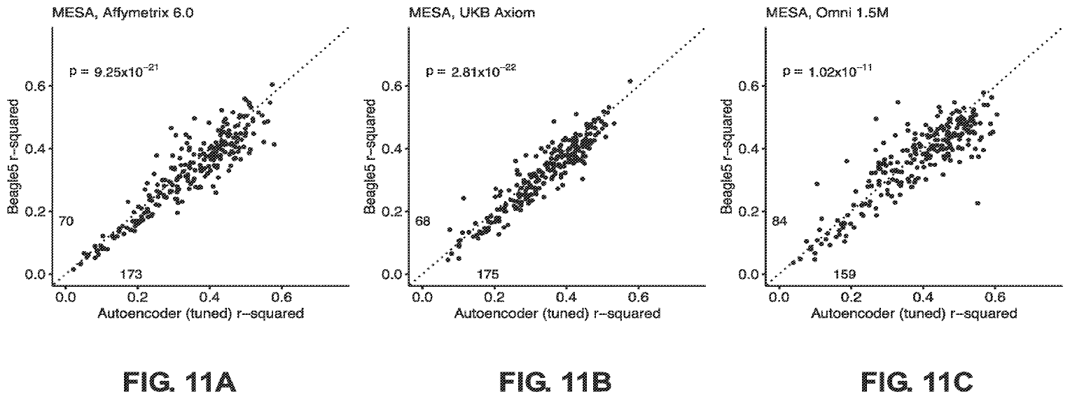

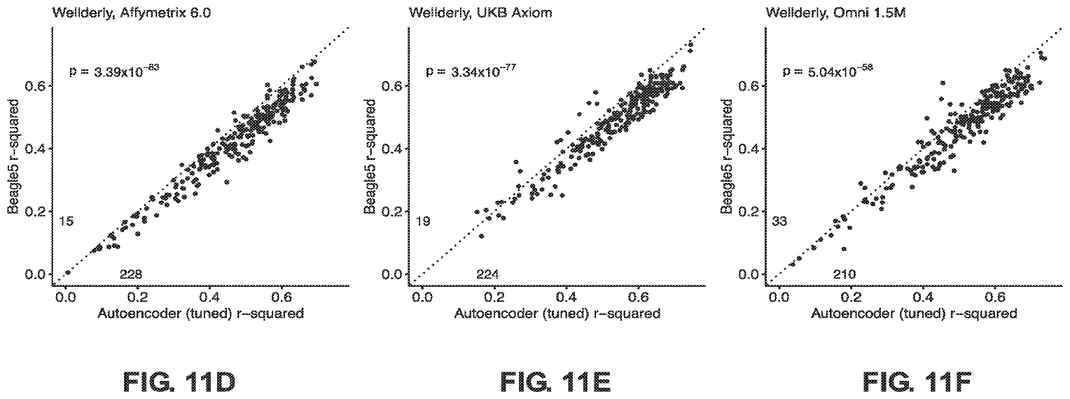

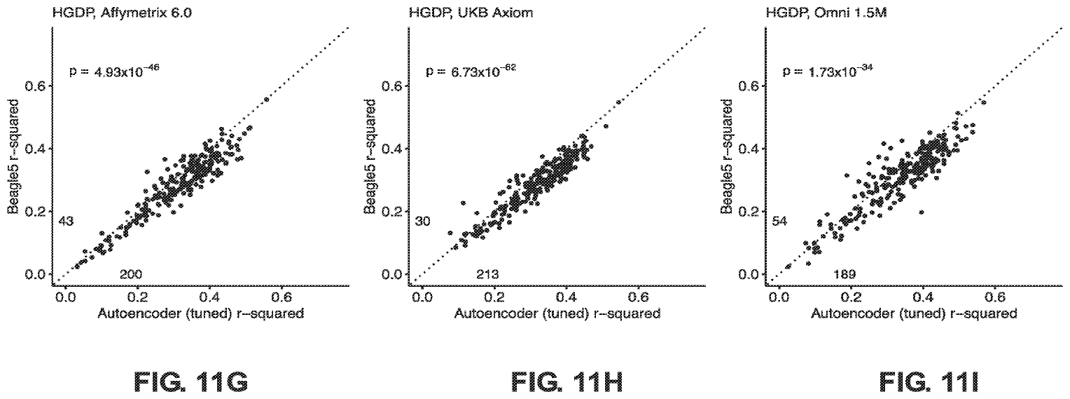

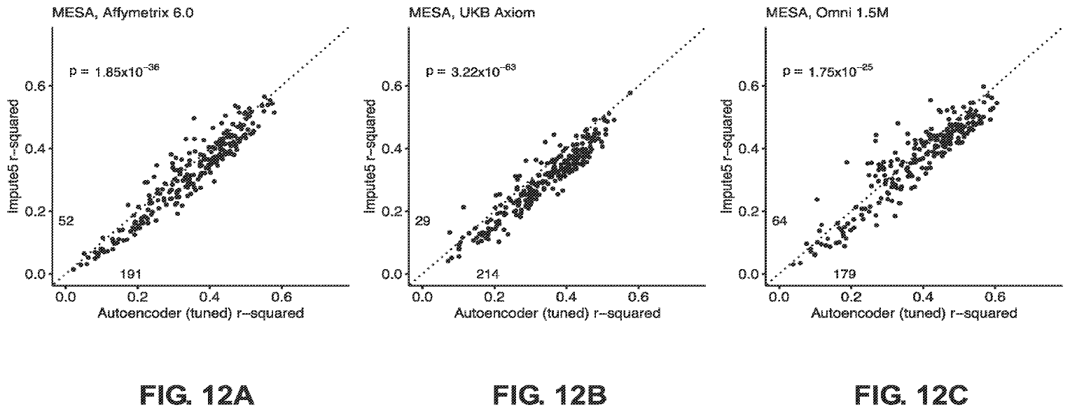

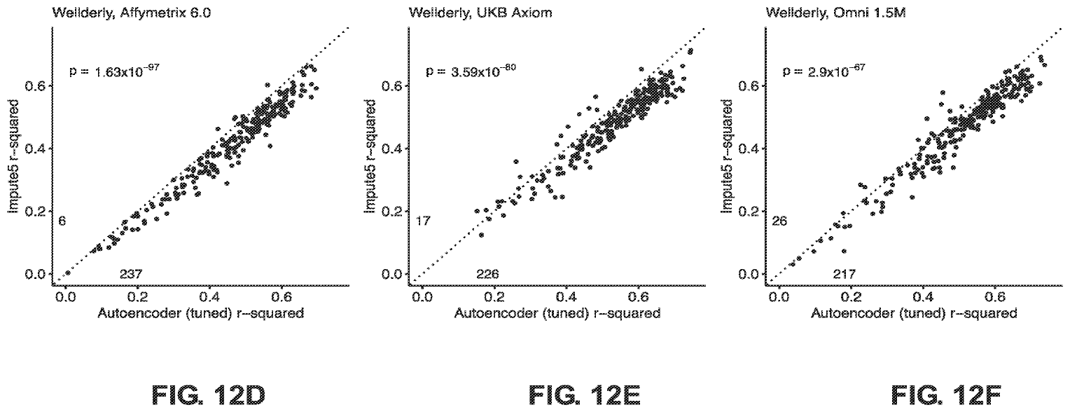

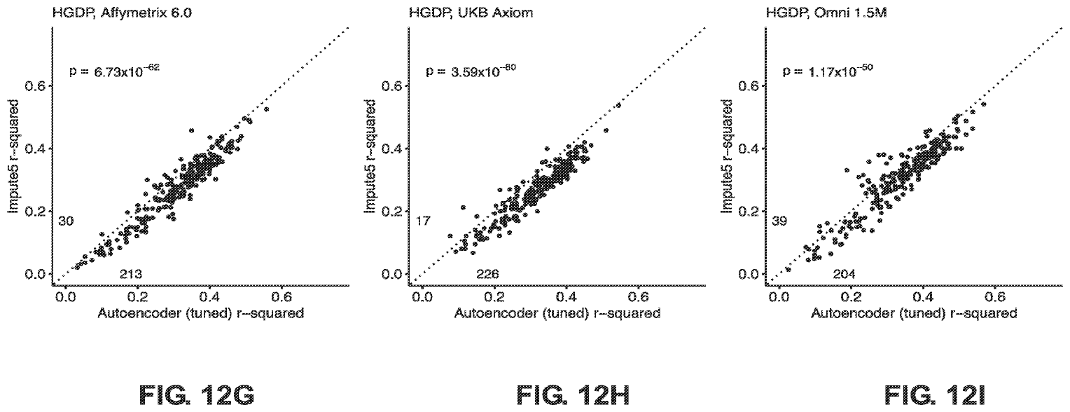

After tuning, autoencoder performance surpassed HMM-based imputation performance across all imputation methods, independent test datasets, and genotyping array marker sets. At a minimum, autoencoders surpassed HMM-based imputation performance in >62% of chromosome 22 genomic segments (two proportion test p=1.02×10⁻¹¹) (Minimac4: FIG. 3, Beagle5 and Eagle5: FIG. 11 and FIG. 12). Overall, the optimized autoencoders exhibited superior performance with an average r-squared of 0.395±0.007 vs 0.374±0.007 for Minimac4 (Wilcoxon rank sum test p=0.007), 0.364±0.007 for Beagle5 (Wilcoxon rank sum test p=1.53*104), and 0.358±0.007 for Impute5 (Wilcoxon rank sum test p=2.01*10-5) (Table 4). This superiority was robust to the marker sets tested, with the mean r-squared per genomic segment for autoencoders being 0.373±0.008, 0.399±0.007, and 0.414±0.008 versus 0.352±0.008, 0.370±0.006, and 0.400±0.007 for Minimac4 using Affymetrix 6.0, UKB Axiom, and Omni 1.5M marker sets (Wilcoxon rank-sums test p-value=0.029, 1.99*104, and 0.087, respectively). Detailed comparisons to Beagle5 and Eagle5 are presented in FIG. 11 and FIG. 12.

TABLE 4
Performance comparisons between tuned autoencoder (AE) and
HMM-based imputation tools (Minimac4, Beagle5, and Impute5)

| | | | | Affymetrix | UKB | Omni | |
|---|---|---|---|---|---|---|---|
| | MESA | Wellderly | HGDP | 6.0 | Axiom | 1.5M | Combined |
| AE | 0.355 ± | 0.505 ± | 0.327 ± | 0.373 ± | 0.399 ± | 0.414 ± | 0.396 ± |
| (tuned) | 0.007 | 0.008 | 0.006 | 0.008 | 0.007 | 0.008 | 0.007 |
| AE | 0.303 ± | 0.470 ± | 0.285 ± | 0.339 ± | 0.356 ± | 0.362 ± | 0.352 ± |
| (untuned) | 0.008*** | 0.009* | 0.006*** | 0.008* | 0.007*** | 0.008*** | 0.008*** |
| Minimac4 | 0.337 ± | 0.471 ± | 0.314 ± | 0.352 ± | 0.370 ± | 0.400 ± | 0.374 ± |
| | 0.007* | 0.008** | 0.006 | 0.008* | 0.006** | 0.007 | 0.007* |
| Beagle5 | 0.336 ± | 0.460 ± | 0.296 ± | 0.342 ± | 0.367 ± | 0.384 ± | 0.364 ± |
| | 0.007* | 0.008*** | 0.005*** | 0.007** | 0.006*** | 0.007** | 0.007** |
| Impute5 | 0.326 ± | 0.458 ± | 0.289 ± | 0.336 ± | 0.354 ± | 0.383 ± | 0.358 ± |
| | 0.007* | 0.008*** | 0.006*** | 0.008** | 0.006*** | 0.008** | 0.007*** |

Average r-squared per variant was extracted from each genomic segment of chromosome 22.
Wilcoxon rank-sum tests was applied to compare the HMM-based tools to the reference untuned autoencoder (AE).
*represents p-values ≤ 0.05,
**indicates p-values ≤ 0.001, and
***indicates p-values ≤ 0.0001.

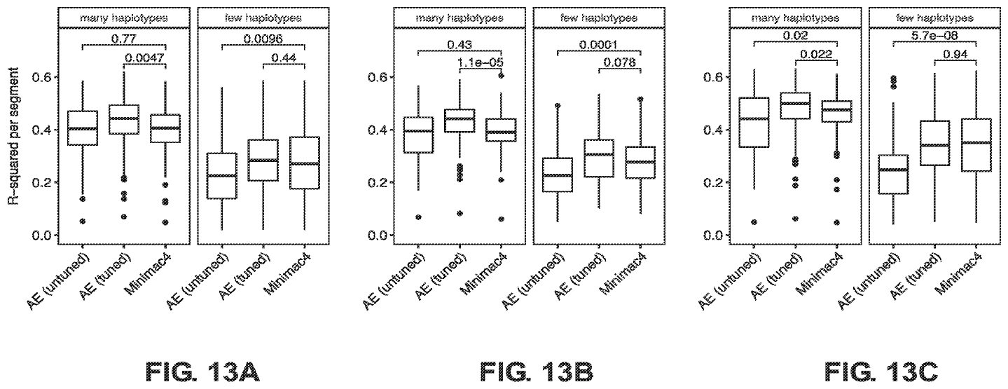

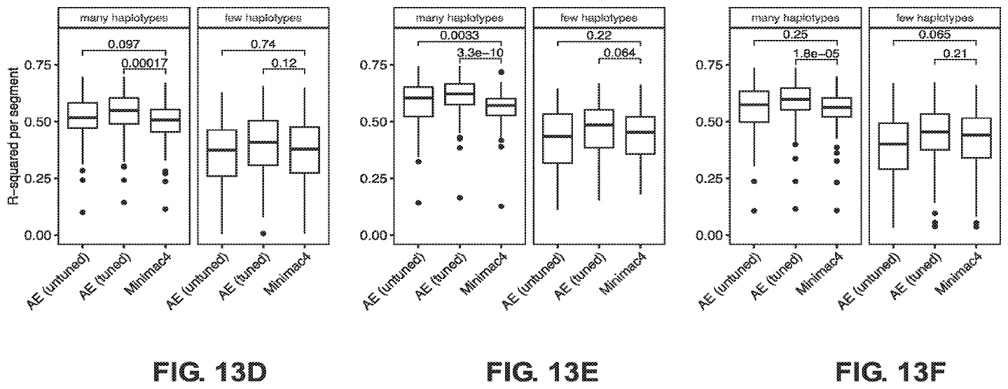

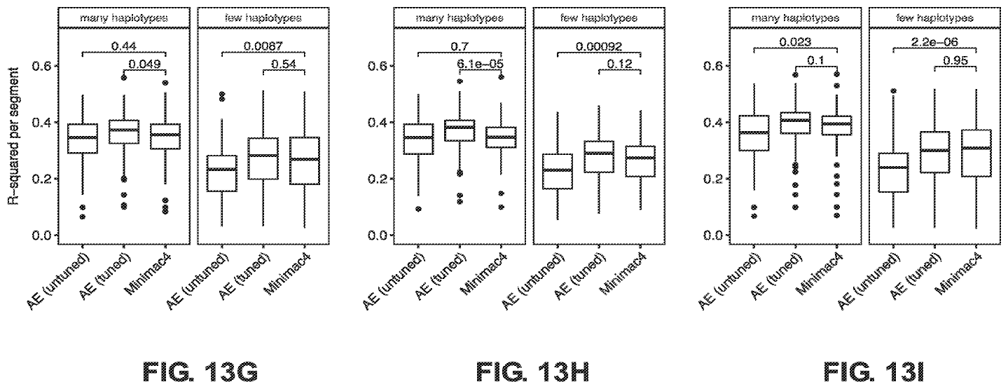

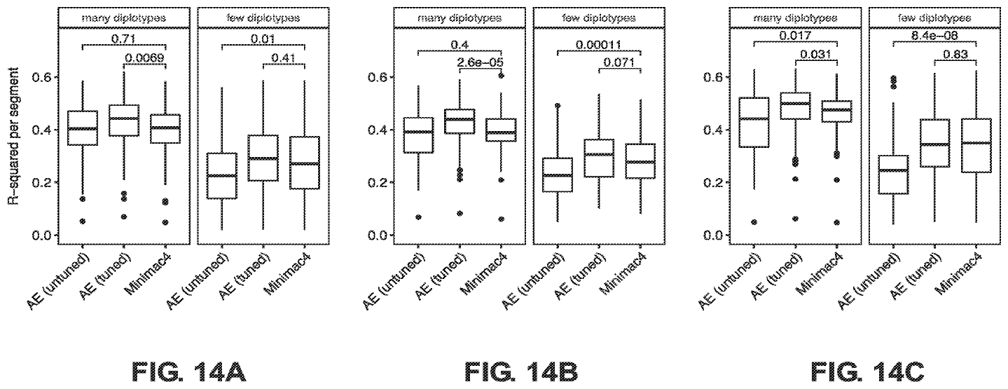

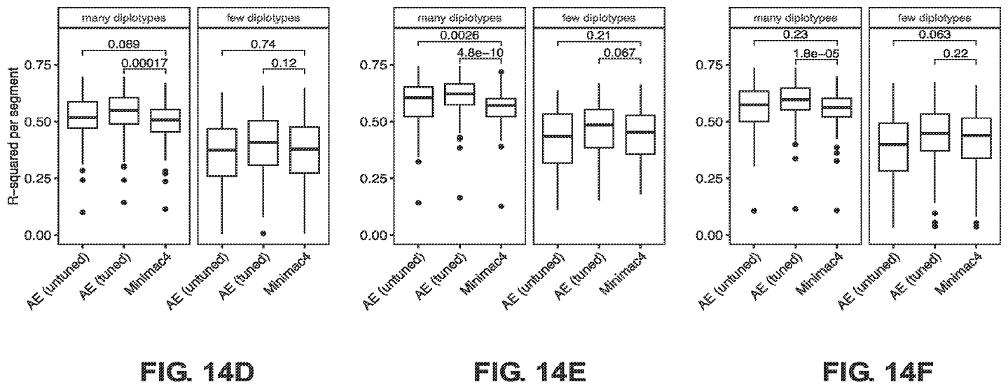

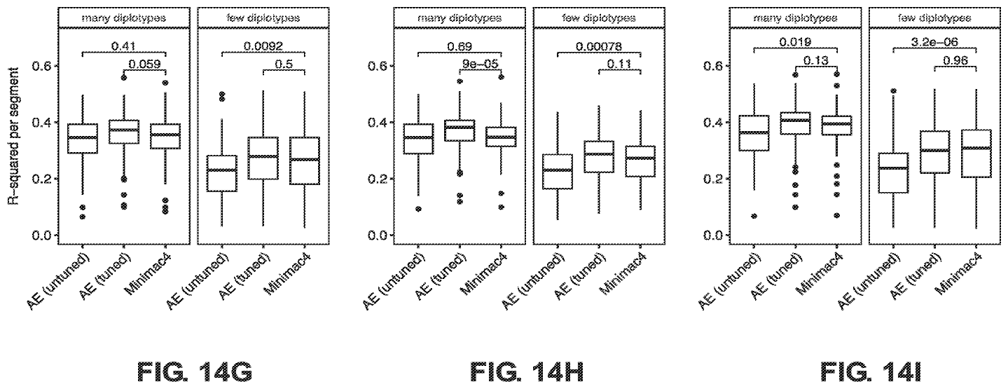

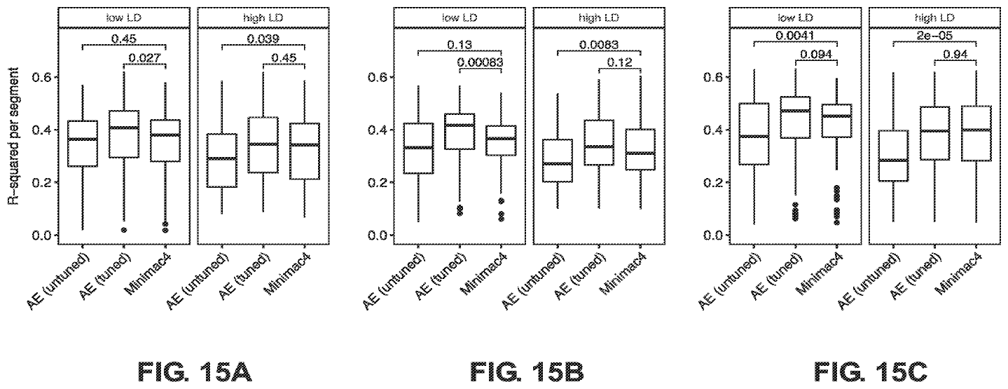

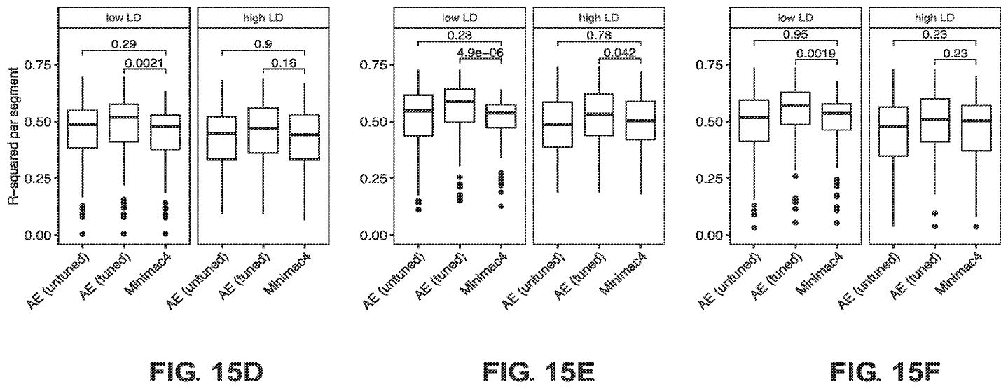

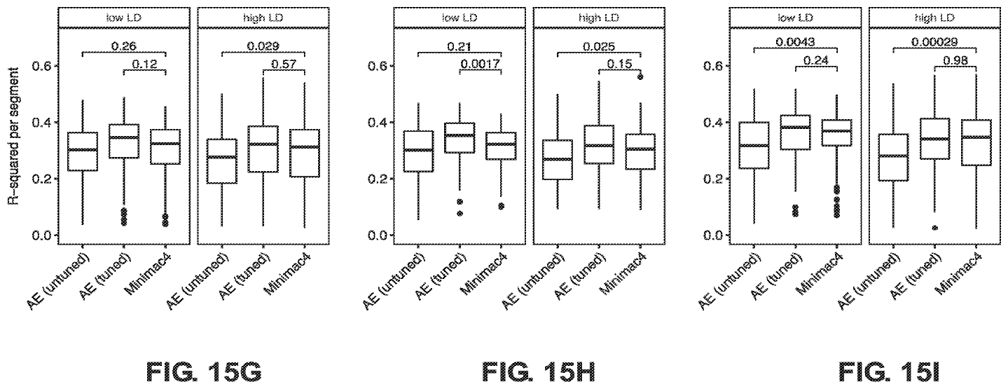

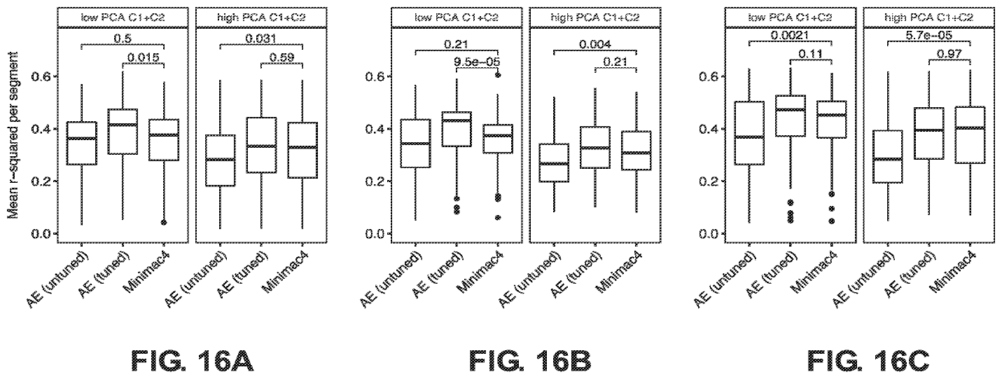

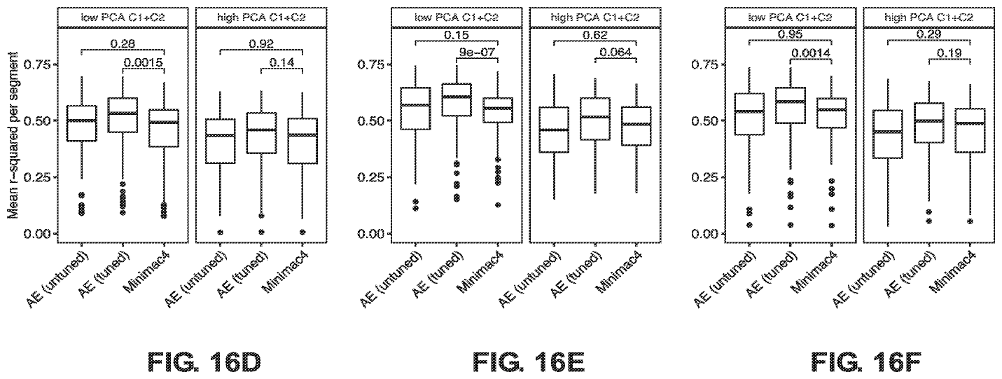

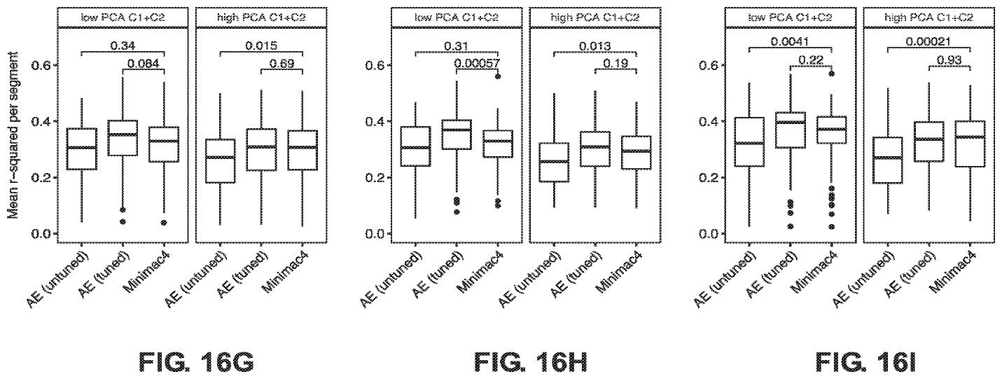

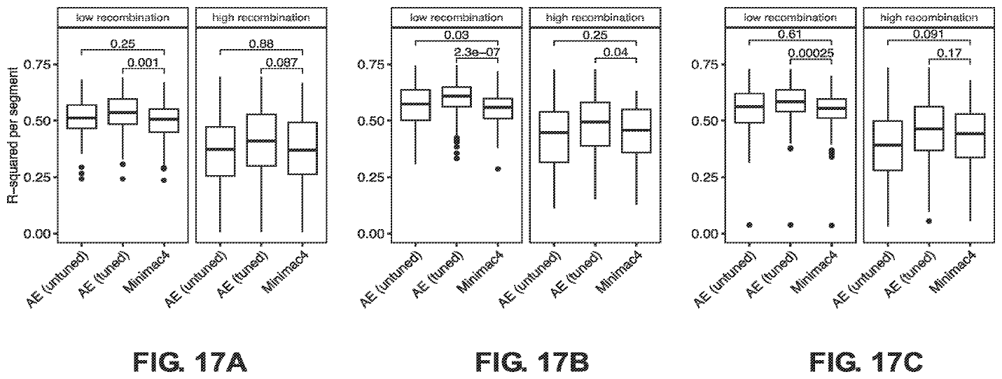

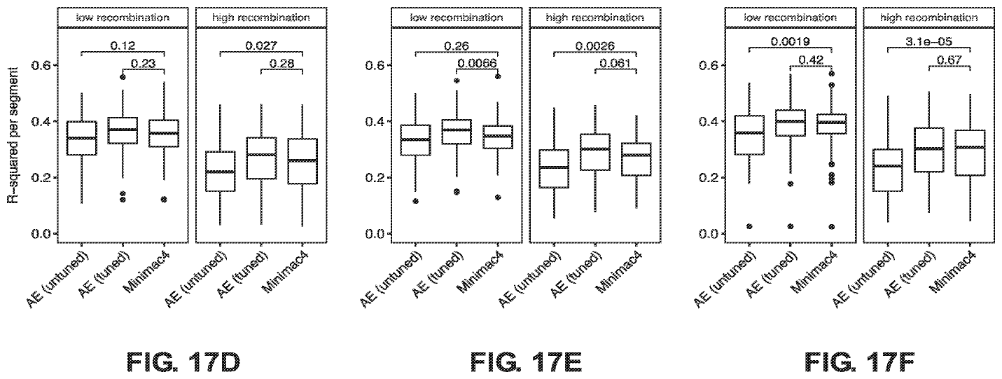

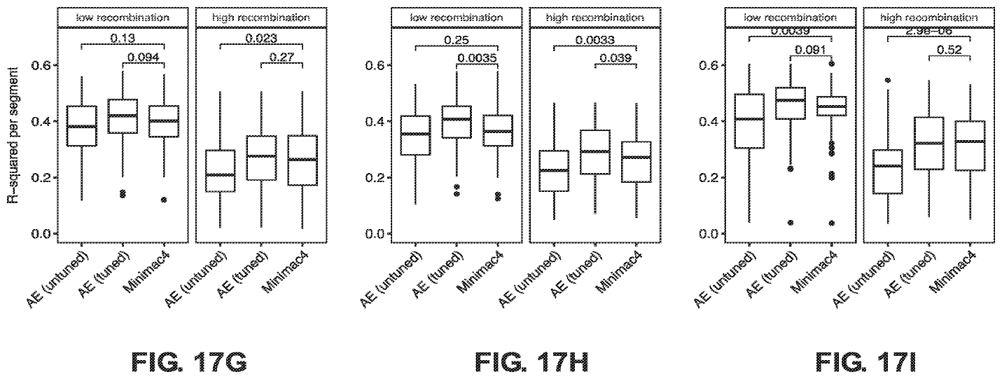

Tuning improved performance of the autoencoders across all genomic segments, generally improving the superiority of autoencoders relative to HMM-based approaches in genomic segments with complex haplotype structures while equalizing performance relative to HMM-based approaches in genomic segments with more simple LD structures (as described herein, by the number of unique haplotypes: FIG. 13, diplotypes: FIG. 14, average pairwise LD: FIG. 15, proportion variance explained: FIG. 16). Concordantly, genomic segments with higher recombination rates exhibited the largest degree of improvement with tuning (FIG. 17). Use of the augmented reference panel did not improve HMM-based imputation, having no influence on Minimac4 performance (original overall r-squared of 0.374±0.007 versus 0.363±0.007 after augmentation, Wilcoxon rank-sum test p=0.0917), and significantly degrading performance of Beagle5 and Impute5 (original r-squared of 0.364±0.007 and 0.358±0.007 versus 0.349±0.006 and 0.324±0.007 after augmentation, p=0.026 and p=1.26*10⁻⁴ respectively). Summary statistics for these comparisons are available in Table 5.

TABLE 5
Performance comparisons between tuned autoencoder (AE) and HMM-based imputation tools
(Minimac4, Beagle5, and Impute5) after applying data augmentation to HMM-based tools.

| | | | | Affymetrix | | | |
|---|---|---|---|---|---|---|---|
| | MESA | Wellderly | HGDP | 6.0 | UKB Axiom | Omni 1.5M | Combined |
| AE (tuned) vs | 1.36e−04* | 3.49e−06* | 1.18e−03* | 6.05e−04* | 6.98e−08* | 1.95e−03* | 3.39e−05* |
| Minimac4 | | | | | | | |
| (augmented) | | | | | | | |
| AE (tuned) vs | 1.71e−05* | 1.68e−09* | 2.88e−09* | 1.54e−06* | 3.94e−10* | 4.30e−07* | 2.30e−08* |
| Beagle5 | | | | | | | |
| (augmented) | | | | | | | |
| AE (tuned) vs | 1.24e−09* | 3.15e−15* | 5.28e−15* | 4.41e−11* | 2.47e−18* | 4.90e−10* | 8.64e−14* |
| Impute5 | | | | | | | |
| (augmented) | | | | | | | |
| Minimac4 | 4.91e−02* | 2.07E−01 | 1.03E−01 | 1.74E−01 | 4.36e−02* | 1.13E−01 | 9.17E−02 |
| (original VS | | | | | | | |
| augmented) | | | | | | | |
| Beagle5 | 1.21e−02* | 8.21E−02 | 2.35e−02* | 8.96E−02 | 6.59e−03* | 5.27E−02 | 2.58e−02* |
| (original VS | | | | | | | |
| augmented) | | | | | | | |
| Impute5 | 5.45e−04* | 6.89e−05* | 1.78e−04* | 7.01e−04* | 1.16e−05* | 4.15e−04* | 1.26e−04* |
| (original VS | | | | | | | |
| augmented) | | | | | | | |
| AE (tuned) | 0.355 ± | 0.505 ± | 0.327 ± | 0.373 ± | 0.399 ± | 0.414 ± | 0.396 ± |
| | 0.007 | 0.008 | 0.006 | 0.008 | 0.007 | 0.008 | 0.007 |
| Minimac4 | 0.322 ± | 0.462 ± | 0.303 ± | 0.342 ± | 0.358 ± | 0.388 ± | 0.363 ± |
| (augmented) | 0.007 | 0.008 | 0.006 | 0.008 | 0.006 | 0.007 | 0.007 |
| Beagle5 | 0.316 ± | 0.446 ± | 0.283 ± | 0.327 ± | 0.348 ± | 0.370 ± | 0.349 ± |
| (augmented) | 0.007 | 0.008 | 0.005 | 0.007 | 0.006 | 0.007 | 0.006 |
| Impute5 | 0.294 ± | 0.416 ± | 0.261 ± | 0.302 ± | 0.318 ± | 0.351 ± | 0.324 ± |
| (augmented) | 0.007 | 0.008 | 0.006 | 0.008 | 0.006 | 0.008 | 0.007 |

Wilcoxon rank-sum tests were applied to compare the HMM-based tools to the reference tuned autoencoder (AE).
*represents p-values ≤ 0.05,
** indicates p-values ≤ 0.001, and
*** indicates p-values ≤ 0.0001

### Overall Chromosome 22 Imputation Accuracy.

After merging the results from all genomic segments, the whole chromosome accuracy of autoencoder-based imputation remained superior to all HMM-based imputation tools, across all independent test datasets, and all genotyping array marker sets (Wilcoxon rank-sums test p≤5.55×10⁻⁶⁷). The autoencoder's mean r-squared per variant ranged from 0.363 for HGDP to 0.605 for the Wellderly vs 0.340 to 0.557 for Minimac4, 0.326 to 0.549 for Beagle5, and 0.314 to 0.547 for Eagle5, respectively. Detailed comparisons are presented in in Table 6 and Table 7.

TABLE 6
Whole chromosome level comparisons between autoencoder (AE) and
HMM-based imputation tools (Minimac4, Beagle5, and Impute5)

| | MESA | | | Wellderly | | | HGDP | | |
|---|---|---|---|---|---|---|---|---|---|
| | Affymet | UKB | Omni | Affymet | UKB | Omni | Affymet | UKB | Omni |
| | rix 6.0 | Axiom | 1.5 M | rix 6.0 | Axiom | 1.5 M | rix 6.0 | Axiom | 1.5 M |
| AE (tuned) | 0.410 | 0.395 | 0.452 | 0.537 | 0.605 | 0.586 | 0.363 | 0.364 | 0.392 |
| Minimac4 | 0.390*** | 0.364*** | 0.436*** | 0.500*** | 0.557*** | 0.551*** | 0.350*** | 0.340*** | 0.385*** |
| Beagle5 | 0.383*** | 0.379*** | 0.420*** | 0.484*** | 0.549*** | 0.534*** | 0.326*** | 0.328*** | 0.353*** |
| Impute5 | 0.384*** | 0.356*** | 0.429*** | 0.485*** | 0.547*** | 0.539*** | 0.328*** | 0.314*** | 0.359*** |

Average r-squared per variant was extracted at whole chromosome level.
Wilcoxon rank-sum tests were applied to compare the HMM-based tools to the reference tuned autoencoder (AE).
* represents p-values ≤ 0.05,
** indicates p-values ≤ 0.001, and
***indicates p-values ≤ 0.0001.
Standard errors that are equal or less than 0.001 are not shown.

TABLE 7
Detailed performance comparisons between tuned autoencoder (AE)
and HMM-based imputation tools (Minimac4, Beagle5, and Impute5).

| | Dataset | | | | |
|---|---|---|---|---|---|
| | MESA | | | Wellderly | |
| Array | Affymetrix 6.0 | UKB Axiom | Omni 1.5M | Affymetrix 6.0 | UKB Axiom |
| AE (tuned) | 9.47e−185*** | 0.00e+00*** | 7.22e−89*** | 6.27e−209*** | 0.00e+00*** |
| VS Minimac4 | | | | | |
| AE (tuned) | 0.00e+00*** | 0.00e+00*** | 0.00e+00*** | 0.00e+00*** | 0.00e+00*** |
| vs Beagle5 | | | | | |
| AE (tuned) | 0.00e+00*** | 0.00e+00*** | 5.37e−191*** | 0.00e+00*** | 0.00e+00*** |
| vs Impute5 | | | | | |
| Minimac4 | 1.73e−259*** | 6.06e−16*** | 0.00e+00*** | 2.68e−86*** | 1.65e−11*** |
| vs Beagle5 | | | | | |
| Minimac4 | 4.87e−38*** | 3.59e−48*** | 1.17e−22*** | 3.05e−74*** | 5.94e−15*** |
| vs Impute5 | | | | | |
| Beagle5 vs | 1.92e−96*** | 2.75e−09*** | 1.23e−175*** | 3.65E−01 | 1.98E−01 |
| Impute5 | | | | | |
| AE (tuned) | 0.410 ± 0.001 | 0.395 ± 0.001 | 0.452 ± 0.001 | 0.537 ± 0.001 | 0.605 ± 0.001 |
| Minimac4 | 0.390 ± 0.001 | 0.364 ± 0.001 | 0.436 ± 0.001 | 0.500 ± 0.001 | 0.557 ± 0.001 |
| Beagle5 | 0.383 ± 0.001 | 0.379 ± 0.001 | 0.420 ± 0.001 | 0.484 ± 0.001 | 0.549 ± 0.001 |
| Impute5 | 0.384 ± 0.001 | 0.356 ± 0.001 | 0.429 ± 0.001 | 0.485 ± 0.001 | 0.547 ± 0.001 |
| | | Dataset | | | |
| | | Wellderly | HGDP | | |
| | Array | Omni 1.5M | Affymetrix 6.0 | UKB Axiom | Omni 1.5M |
| | AE (tuned) | 2.75e−198*** | 2.35e−151*** | 0.00e+00*** | 5.55e−67*** |
| | VS Minimac4 | | | | |
| | AE (tuned) | 0.00e+00*** | 0.00e+00*** | 0.00e+00*** | 0.00e+00*** |
| | vs Beagle5 | | | | |
| | AE (tuned) | 0.00e+00*** | 0.00e+00*** | 0.00e+00*** | 0.00e+00*** |
| | vs Impute5 | | | | |
| | Minimac4 | 6.87e−64*** | 0.00e+00*** | 2.62e−185*** | 0.00e+00*** |
| | vs Beagle5 | | | | |
| | Minimac4 | 1.00e−25*** | 9.43e−261*** | 0.00e+00*** | 1.73e−251*** |
| | vs Impute5 | | | | |
| | Beagle5 vs | 9.53e−09*** | 1.22e−25*** | 2.61e−17*** | 8.36e−57*** |
| | Impute5 | | | | |
| | AE (tuned) | 0.586 ± 0.001 | 0.363 ± 0.001 | 0.364 ± 0.001 | 0.392 ± 0.001 |
| | Minimac4 | 0.551 ± 0.001 | 0.350 ± 0.001 | 0.340 ± 0.001 | 0.385 ± 0.001 |
| | Beagle5 | 0.534 ± 0.001 | 0.326 ± 0.001 | 0.328 ± 0.001 | 0.353 ± 0.001 |
| | Impute5 | 0.539 ± 0.001 | 0.328 ± 0.001 | 0.314 ± 0.001 | 0.359 ± 0.001 |

Validation accuracies were stratified by dataset (MESA, Wellderly, HGDP) and genotype array platform (Affymetrix 6.0, UKB Axiom, Omni 1.5M).
Wilcoxon rank-sum tests were applied to compare the HMM-based tools to the reference tuned autoencoder (AE).
* represents p-values ≤ 0.05,
** indicates p-values ≤ 0.001, and
***indicates p-values ≤ 0.0001

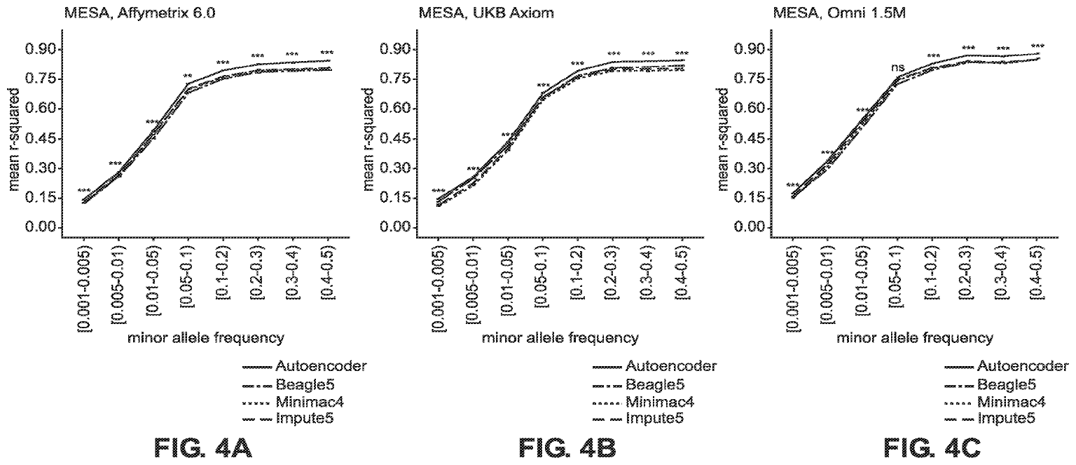

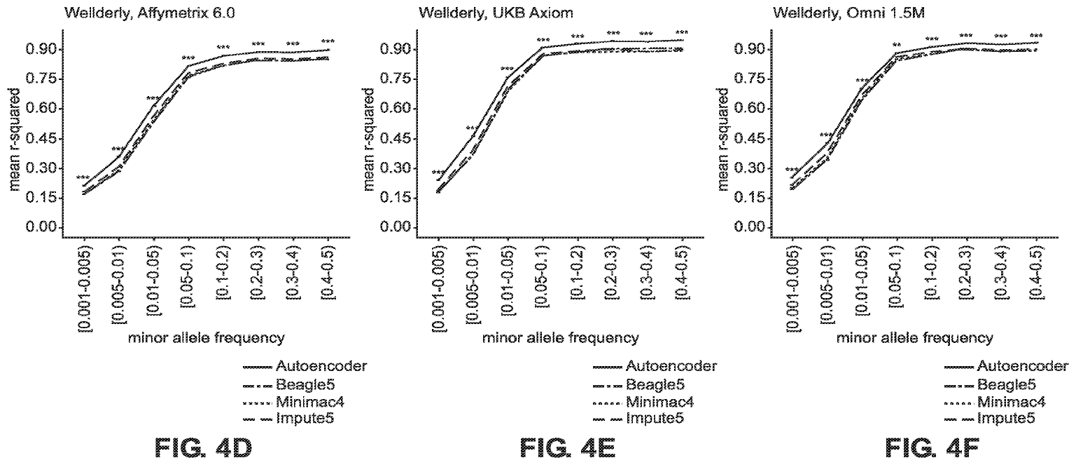

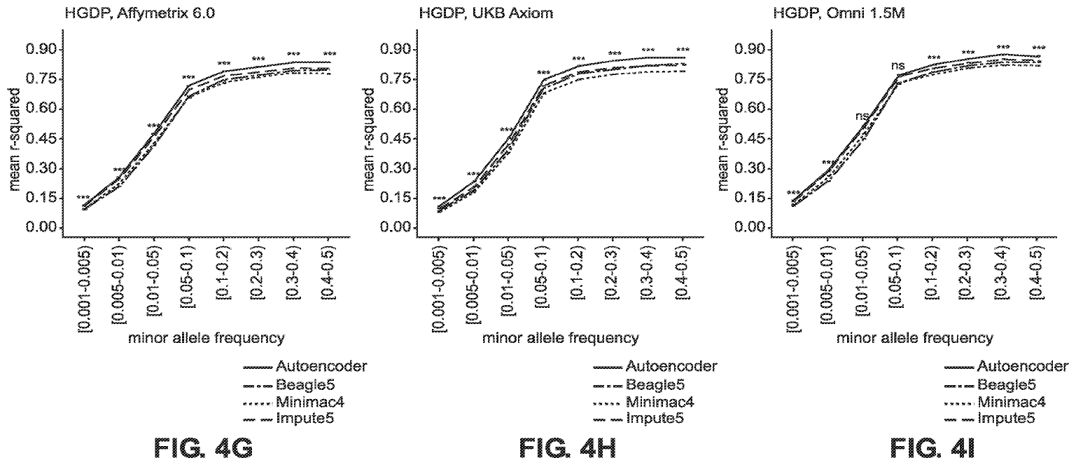

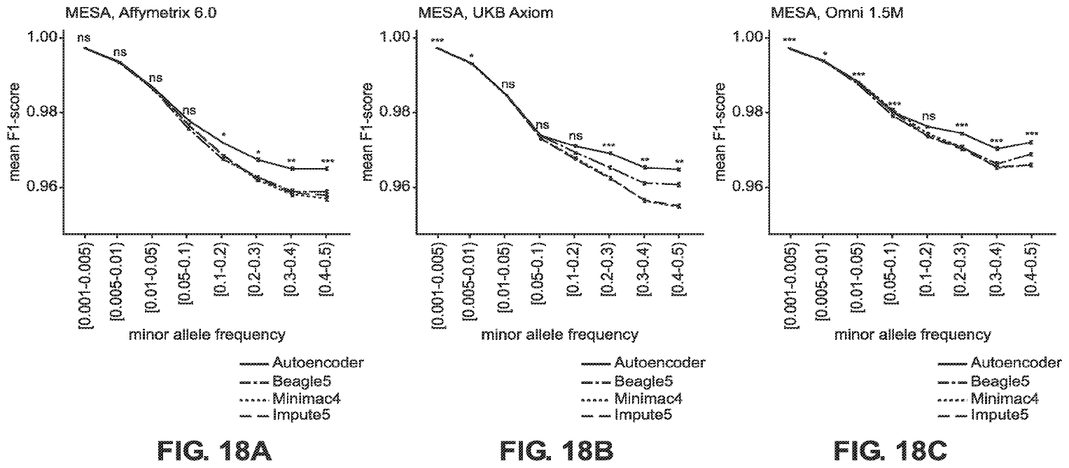

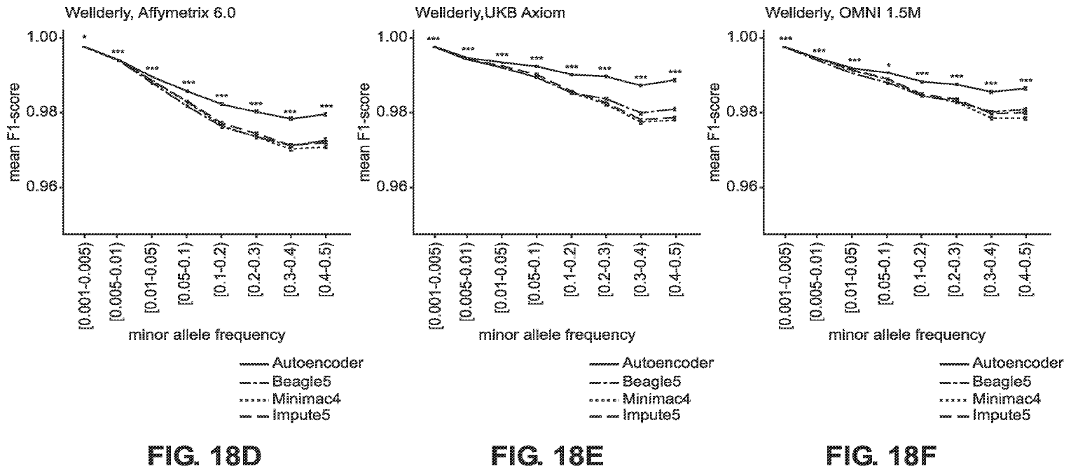

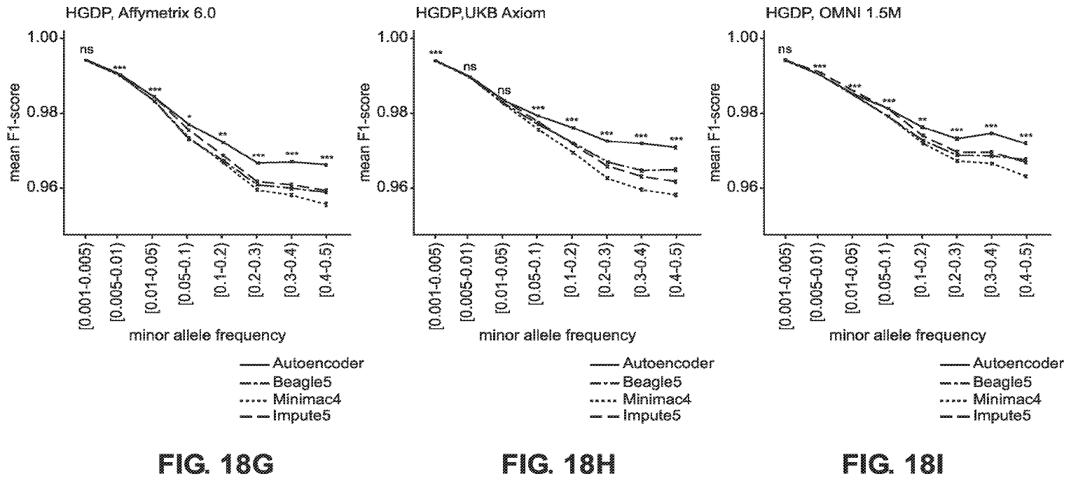

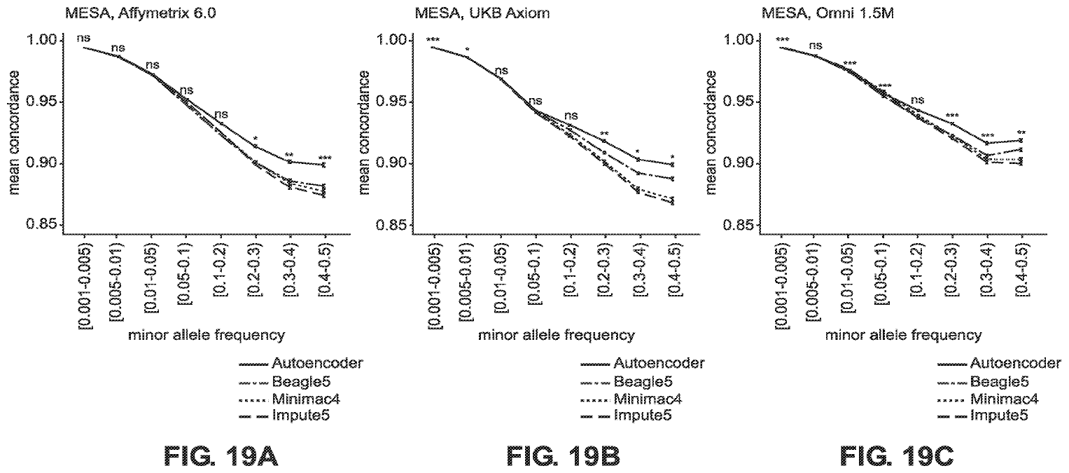

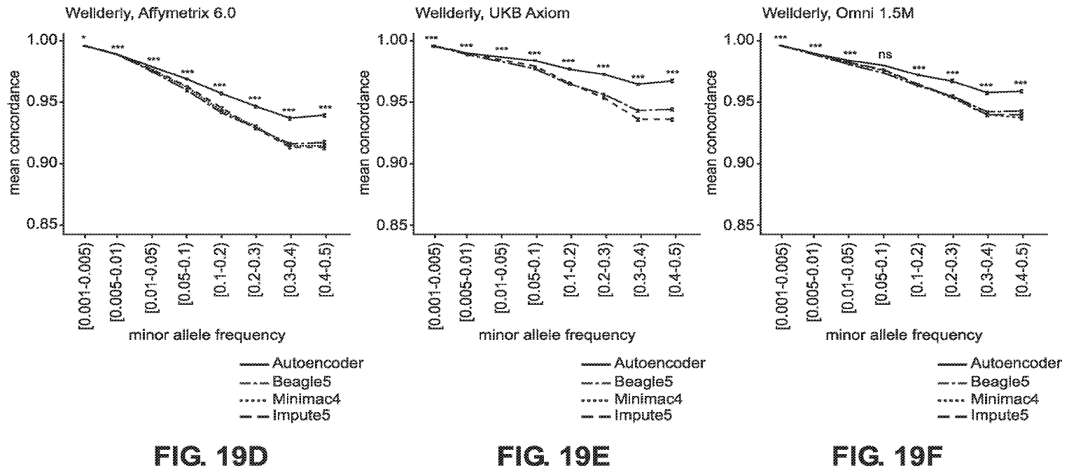

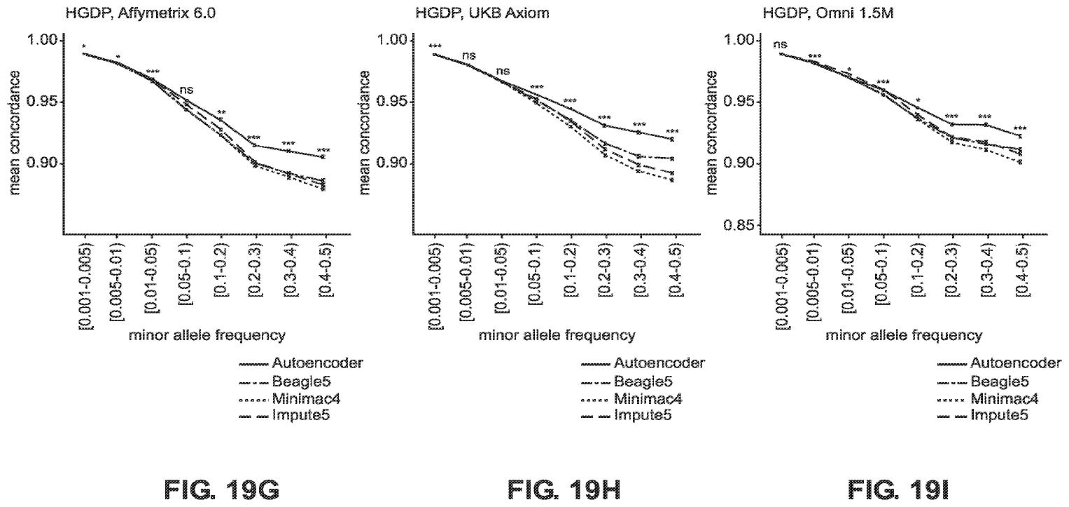

Further, when imputation accuracy is stratified by MAF bins, the autoencoders maintain superiority across all MAF bins by nearly all test dataset and genotyping array marker sets (FIG. 4, and Table 8). Concordantly, autoencoder imputation accuracy is similarly superior when measured with F1-scores (FIG. 18) and concordance (FIG. 19), though these metrics are less sensitive at capturing differences in rare variant imputation accuracy.

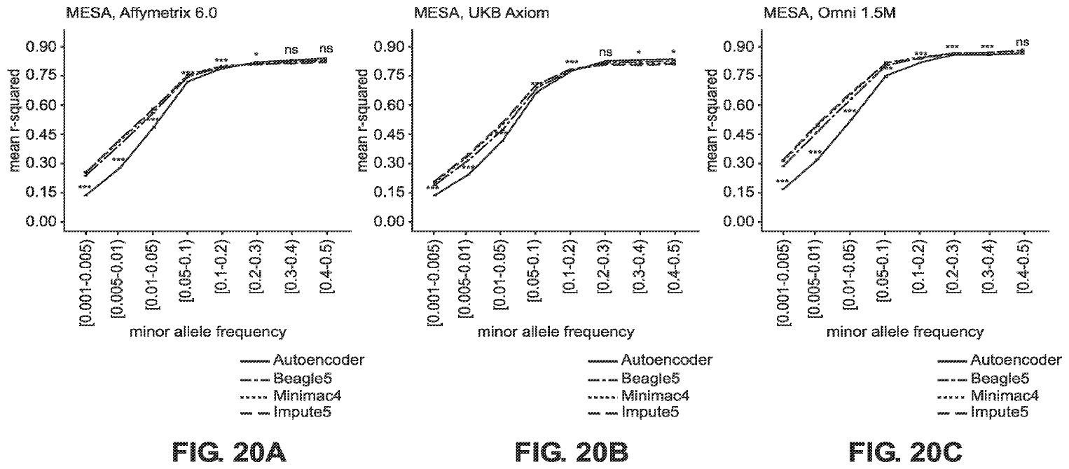

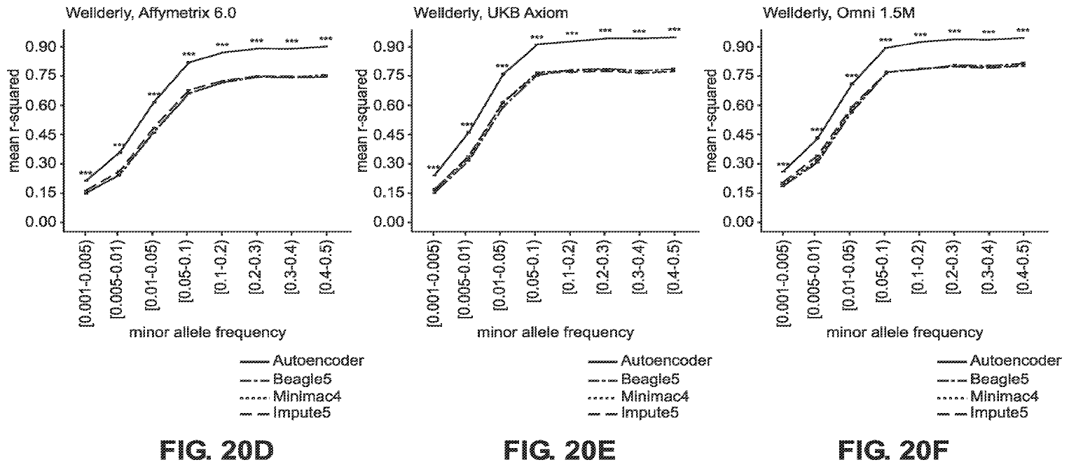

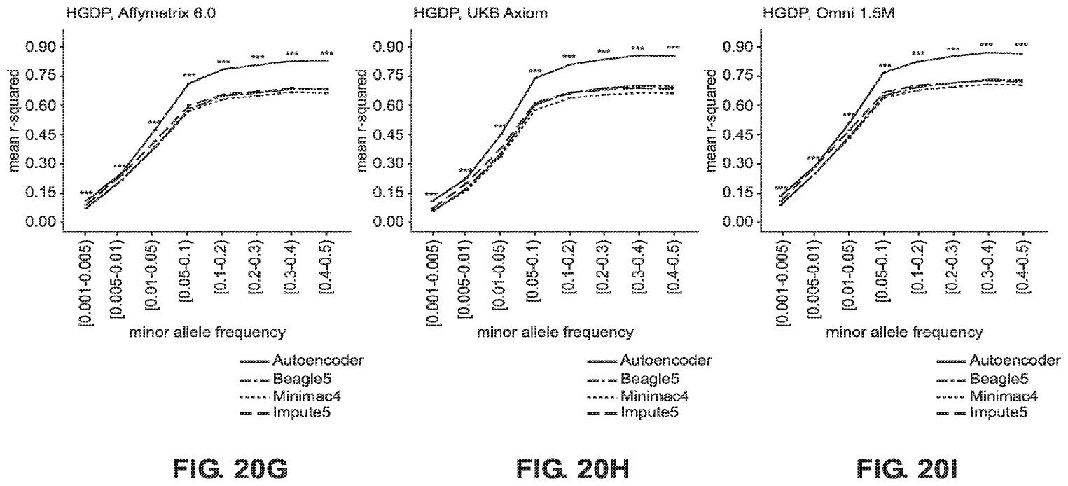

When the reference panel of the HMM-based tools was upgraded with the more expansive TOPMed cohort, the superior performance of the HRC-trained autoencoder was still sustained across all datasets except for MESA (FIG. 20). Given that MESA is a sub-cohort of the TOPMed cohort, the inventors evaluated the possibility of residual data leakage after the removal of MESA from the TOPMed cohort and found that 44 MESA individuals were duplicated in other TOPMed cohorts, 182 MESA individuals had a first degree relative in other TOPMed cohorts, and >92% of MESA individuals had at least one second degree relative in other TOPMed cohorts, resulting in improved imputation performance. Notably, across the most diverse and truly independent HGDP validation dataset, the autoencoder displays superior performance despite only being exposed to training on the less diverse HRC reference cohort.

TABLE 8
Detailed performance comparisons between tuned autoencoder (AE)
and HMM-based imputation tools (Minimac4, Beagle5, and Impute5).

| | | | AE (tuned) | AE (tuned) | AE (tuned) | Minimac4 |
|---|---|---|---|---|---|---|
| Dataset | Array | MAF | vs Minimac4 | vs Beagle5 | vs Impute5 | vs Beagle5 |
| MESA | Affymetrix | [0.001- | 3.84e−306*** | 0.00e+00*** | 0.00e+00*** | 0.00e+00*** |
| | 6.0 | 0.005) | | | | |
| MESA | Affymetrix | [0.005- | 1.37e−48*** | 3.39e−280*** | 8.96e−86*** | 3.12e−127*** |
| | 6.0 | 0.01) | | | | |
| MESA | Affymetrix | [0.01- | 2.62e−48*** | 6.14e−119*** | 3.54e−72*** | 4.61e−23*** |
| | 6.0 | 0.05) | | | | |
| MESA | Affymetrix | [0.05- | 2.11e−05*** | 4.44e−49*** | 2.36e−04** | 3.26e−23*** |
| | 6.0 | 0.1) | | | | |
| MESA | Affymetrix | [0.1- | 1.17e−15*** | 2.04e−50*** | 3.36e−09*** | 6.41e−11*** |
| | 6.0 | 0.2) | | | | |
| MESA | Affymetrix | [0.2- | 1.02e−16*** | 5.61e−25*** | 6.87e−09*** | 5.09E−02 |
| | 6.0 | 0.3) | | | | |
| MESA | Affymetrix | [0.3- | 2.41e−19*** | 3.04e−28*** | 2.92e−09*** | 9.05E−02 |
| | 6.0 | 0.4) | | | | |
| MESA | Affymetrix | [0.4- | 2.46e−18*** | 7.67e−23*** | 3.85e−11*** | 3.80E−01 |
| | 6.0 | 0.5) | | | | |
| MESA | UKB | [0.001- | 0.00e+00*** | 0.00e+00*** | 0.00e+00*** | 0.00e+00*** |
| | Axiom | 0.005) | | | | |
| MESA | UKB | [0.005- | 7.45e−142*** | 7.16e−191*** | 4.41e−242*** | 2.76e−23*** |
| | Axiom | 0.01) | | | | |
| MESA | UKB | [0.01- | 2.85e−128*** | 6.38e−41*** | 1.57e−181*** | 1.76e−12*** |
| | Axiom | 0.05) | | | | |
| MESA | UKB | [0.05- | 5.91e−21*** | 4.88e−09*** | 2.14e−23*** | 2.68e−03* |
| | Axiom | 0.1) | | | | |
| MESA | UKB | [0.1- | 1.12e−42*** | 2.66e−11*** | 5.58e−37*** | 9.55e−11*** |
| | Axiom | 0.2) | | | | |
| MESA | UKB | [0.2- | 8.25e−59*** | 1.62e−15*** | 5.06e−54*** | 2.23e−14*** |
| | Axiom | 0.3) | | | | |
| MESA | UKB | [0.3- | 8.13e−81*** | 7.00e−14*** | 3.82e−72*** | 3.98e−26*** |
| | Axiom | 0.4) | | | | |
| MESA | UKB | [0.4- | 5.60e−82*** | 8.03e−12*** | 1.69e−79*** | 6.34e−30*** |
| | Axiom | 0.5) | | | | |
| MESA | Omni 1.5M | [0.001- | 6.78e−179*** | 0.00e+00*** | 0.00e+00*** | 0.00e+00*** |
| | | 0.005) | | | | |
| MESA | Omni 1.5M | [0.005- | 1.37e−23*** | 7.31e−253*** | 5.27e−53*** | 6.68e−147*** |
| | | 0.01) | | | | |
| MESA | Omni 1.5M | [0.01- | 1.51e−05*** | 6.15e−118*** | 9.61e−14*** | 2.01e−77*** |
| | | 0.05) | | | | |
| MESA | Omni 1.5M | [0.05- | 8.96E−01 | 2.53e−52*** | 1.91E−01 | 2.25e−48*** |
| | | 0.1) | | | | |
| MESA | Omni 1.5M | [0.1- | 7.72e−19*** | 3.14e−57*** | 5.03e−09*** | 5.40e−11*** |
| | | 0.2) | | | | |
| MESA | Omni 1.5M | [0.2- | 2.53e−32*** | 2.68e−65*** | 6.69e−18*** | 8.66e−06*** |
| | | 0.3) | | | | |
| MESA | Omni 1.5M | [0.3- | 4.87e−32*** | 3.52e−34*** | 5.66e−20*** | 8.88E−01 |
| | | 0.4) | | | | |
| MESA | Omni 1.5M | [0.4- | 2.05e−53*** | 1.11e−22*** | 1.92e−38*** | 3.17e−08*** |
| | | 0.5) | | | | |
| Wellderly | Affymetrix | [0.001- | 0.00e+00*** | 0.00e+00*** | 0.00e+00*** | 0.00e+00*** |
| | 6.0 | 0.005) | | | | |
| Wellderly | Affymetrix | [0.005 | 9.46e−58*** | 4.82e−140*** | 2.37e−167*** | 1.84e−26*** |
| | 6.0 | -0.01) | | | | |
| Wellderly | Affymetrix | [0.01- | 4.01e−43*** | 2.50e−149*** | 1.02e−88*** | 6.52e−34*** |
| | 6.0 | 0.05) | | | | |
| Wellderly | Affymetrix | [0.05- | 4.80e−19*** | 2.25e−55*** | 7.24e−16*** | 7.05e−12*** |
| | 6.0 | 0.1) | | | | |
| Wellderly | Affymetrix | [0.1- | 1.08e−46*** | 4.14e−53*** | 3.92e−34*** | 2.40E−01 |
| | 6.0 | 0.2) | | | | |
| Wellderly | Affymetrix | [0.2- | 7.27e−35*** | 4.70e−38*** | 1.12e−23*** | 5.90E−01 |
| | 6.0 | 0.3) | | | | |
| Wellderly | Affymetrix | [0.3- | 1.93e−49*** | 3.99e−32*** | 1.43e−35*** | 7.35e−03* |
| | 6.0 | 0.4) | | | | |
| Wellderly | Affymetrix | [0.4- | 8.77e−51*** | 7.23e−32*** | 5.15e−31*** | 3.34e−03* |
| | 6.0 | 0.5) | | | | |
| Wellderly | UKB | [0.001- | 0.00e+00*** | 0.00e+00*** | 0.00e+00*** | 1.56e−98*** |
| | Axiom | 0.005) | | | | |
| Wellderly | UKB | [0.005- | 1.42e−78*** | 3.64e−137*** | 4.44e−134*** | 8.25e−14*** |
| | Axiom | 0.01) | | | | |
| Wellderly | UKB | [0.01- | 3.20e−43*** | 4.20e−124*** | 1.06e−54*** | 5.93e−22*** |
| | Axiom | 0.05) | | | | |
| Wellderly | UKB | [0.05- | 1.63e−62*** | 1.70e−80*** | 1.47e−32*** | 6.88e−03* |
| | Axiom | 0.1) | | | | |
| Wellderly | UKB | [0.1- | 1.21e−141*** | 4.36e−96*** | 4.21e−87*** | 1.88e−04** |
| | Axiom | 0.2) | | | | |
| Wellderly | UKB | [0.2- | 4.84e−219*** | 7.82e−121*** | 4.78e−154*** | 9.07e−15*** |
| | Axiom | 0.3) | | | | |
| Wellderly | UKB | [0.3- | 5.32e−257*** | 3.33e−135*** | 1.46e−196*** | 1.38e−21*** |
| | Axiom | 0.4) | | | | |
| Wellderly | UKB | [0.4- | 0.00e+00*** | 5.32e−156*** | 1.60e−244*** | 1.62e−34*** |
| | Axiom | 0.5) | | | | |
| Wellderly | Omni 1.5M | [0.001- | 0.00e+00*** | 0.00e+00*** | 0.00e+00*** | 2.69e−183*** |
| | | 0.005) | | | | |
| Wellderly | Omni 1.5M | [0.005 | 1.20e−46*** | 1.49e−125*** | 1.09e−102*** | 8.69e−25*** |
| | | -0.01) | | | | |
| Wellderly | Omni 1.5M | [0.01- | 1.67e−13*** | 1.07e−93*** | 1.95e−25*** | 2.72e−36*** |
| | | 0.05) | | | | |
| Wellderly | Omni 1.5M | [0.05- | 5.40e−07*** | 6.60e−59*** | 8.37e−04** | 1.59e−26*** |
| | | 0.1) | | | | |
| Wellderly | Omni 1.5M | [0.1- | 2.89e−42*** | 1.73e−55*** | 1.08e−22*** | 4.85e−02* |
| | | 0.2) | | | | |
| Wellderly | Omni 1.5M | [0.2- | 2.56e−68*** | 6.21e−68*** | 9.93e−47*** | 8.71E−01 |
| | | 0.3) | | | | |
| Wellderly | Omni 1.5M | [0.3- | 3.99e−89*** | 1.54e−58*** | 3.55e−68*** | 1.15e−04** |
| | | 0.4) | | | | |
| Wellderly | Omni 1.5M | [0.4- | 1.88e−101*** | 5.27e−60*** | 2.12e−68*** | 6.34e−07*** |
| | | 0.5) | | | | |
| HGDP | Affymetrix | [0.001- | 0.00e+00*** | 0.00e+00*** | 0.00e+00*** | 0.00e+00*** |
| | 6.0 | 0.005) | | | | |
| HGDP | Affymetrix | [0.005- | 9.42e−37*** | 0.00e+00*** | 1.41e−180*** | 1.23e−172*** |
| | 6.0 | 0.01) | | | | |
| HGDP | Affymetrix | [0.01- | 1.27e−19*** | 5.35e−251*** | 5.51e−161*** | 3.40e−124*** |
| | 6.0 | 0.05) | | | | |
| HGDP | Affymetrix | [0.05- | 5.84e−07*** | 1.05e−90*** | 3.49e−67*** | 5.86e−46*** |
| | 6.0 | 0.1) | | | | |
| HGDP | Affymetrix | [0.1- | 4.01e−09*** | 6.45e−78*** | 6.97e−76*** | 1.44e−32*** |
| | 6.0 | 0.2) | | | | |
| HGDP | Affymetrix | [0.2- | 2.52e−13*** | 1.54e−56*** | 2.43e−64*** | 9.52e−15*** |
| | 6.0 | 0.3) | | | | |
| HGDP | Affymetrix | [0.3- | 5.85e−16*** | 6.47e−64*** | 7.68e−72*** | 2.25e−15*** |
| | 6.0 | 0.4) | | | | |
| HGDP | Affymetrix | [0.4- | 2.89e−16*** | 7.59e−40*** | 9.69e−63*** | 7.01e−06*** |
| | 6.0 | 0.5) | | | | |
| HGDP | UKB | [0.001- | 0.00e+00*** | 0.00e+00*** | 0.00e+00*** | 0.00e+00*** |
| | Axiom | 0.005) | | | | |
| HGDP | UKB | [0.005- | 6.42e−154*** | 0.00e+00*** | 0.00e+00*** | 1.60e−75*** |
| | Axiom | 0.01) | | | | |
| HGDP | UKB | [0.01- | 1.58e−100*** | 7.46e−277*** | 0.00e+00*** | 7.30e−49*** |
| | Axiom | 0.05) | | | | |
| HGDP | UKB | [0.05- | 9.25e−18*** | 8.34e−48*** | 3.52e−139*** | 2.05e−08*** |
| | Axiom | 0.1) | | | | |
| HGDP | UKB | [0.1- | 1.69e−51*** | 1.48e−73*** | 1.93e−253*** | 3.65e−03* |
| | Axiom | 0.2) | | | | |
| HGDP | UKB | [0.2- | 2.31e−71*** | 5.99e−63*** | 4.54e−249*** | 3.50E−01 |
| | Axiom | 0.3) | | | | |
| HGDP | UKB | [0.3- | 1.63e−104*** | 4.84e−81*** | 3.67e−302*** | 9.67e−03* |
| | Axiom | 0.4) | | | | |
| HGDP | UKB | [0.4- | 3.74e−93*** | 5.24e−50*** | 8.92e−262*** | 5.86e−08*** |
| | Axiom | 0.5) | | | | |
| HGDP | Omni 1.5M | [0.001- | 5.46e−226*** | 0.00e+00*** | 0.00e+00*** | 0.00e+00*** |
| | | 0.005) | | | | |
| HGDP | Omni 1.5M | [0.005- | 1.04e−09*** | 0.00e+00*** | 2.39e−110*** | 2.55e−221*** |
| | | 0.01) | | | | |
| HGDP | Omni 1.5M | [0.01- | 4.61E−01 | 7.26e−252*** | 8.61e−99*** | 1.15e−221*** |
| | | 0.05) | | | | |
| HGDP | Omni 1.5M | [0.05- | 1.58E−01 | 5.99e−77*** | 2.18e−41*** | 5.10e−79*** |
| | | 0.1) | | | | |
| HGDP | Omni 1.5M | [0.1- | 1.63e−08*** | 3.95e−95*** | 7.52e−111*** | 1.44e−43*** |
| | | 0.2) | | | | |
| HGDP | Omni 1.5M | [0.2- | 2.18e−14*** | 5.45e−80*** | 1.70e−107*** | 2.92e−25*** |
| | | 0.3) | | | | |
| HGDP | Omni 1.5M | [0.3- | 1.09e−34*** | 1.27e−114*** | 5.18e−157*** | 7.10e−21*** |
| | | 0.4) | | | | |
| HGDP | Omni 1.5M | [0.4- | 2.78e−29*** | 8.21e−50*** | 4.50e−131*** | 1.90e−03* |
| | | 0.5) | | | | |

| | Minimac4 | Beagle5 | | | | |
|---|---|---|---|---|---|---|
| Dataset | vs Impute5 | vs Impute5 | AE (tuned) | Minimac4 | Beagle5 | Impute5 |
| MESA | 1.30e−122*** | 0.00e+00*** | 0.141 ± | 0.128 ± | 0.129 ± | 0.121 ± |
| | | | 0.001 | 0.001 | 0.001 | 0.001 |
| MESA | 1.13e−08*** | 1.22e−68*** | 0.280 ± | 0.266 ± | 0.258 ± | 0.262 ± |
| | | | 0.001 | 0.001 | 0.001 | 0.001 |
| MESA | 8.85e−05*** | 7.81e−09*** | 0.490 ± | 0.467 ± | 0.453 ± | 0.461 ± |
| | | | 0.001 | 0.001 | 0.001 | 0.001 |
| MESA | 7.11E−01 | 3.53e−24*** | 0.728 ± | 0.703 ± | 0.684 ± | 0.698 ± |
| | | | 0.002 | 0.002 | 0.002 | 0.002 |
| MESA | 6.50E−02 | 8.64e−16*** | 0.793 ± | 0.763 ± | 0.753 ± | 0.758 ± |
| | | | 0.002 | 0.002 | 0.002 | 0.002 |
| MESA | 2.67e−02* | 5.57e−05*** | 0.825 ± | 0.794 ± | 0.790 ± | 0.789 ± |
| | | | 0.002 | 0.002 | 0.002 | 0.002 |
| MESA | 1.08e−02* | 3.17e−05*** | 0.834 ± | 0.799 ± | 0.798 ± | 0.795 ± |
| | | | 0.002 | 0.002 | 0.002 | 0.002 |
| MESA | 7.17E−02 | 9.77e−03* | 0.842 ± | 0.806 ± | 0.805 ± | 0.801 ± |
| | | | 0.002 | 0.002 | 0.003 | 0.003 |
| MESA | 1.04e−106*** | 1.10e−95*** | 0.145 ± | 0.117 ± | 0.132 ± | 0.108 ± |
| | | | 0.001 | 0.001 | 0.001 | 0.001 |
| MESA | 8.03e−18*** | 1.28E−01 | 0.255 ± | 0.226 ± | 0.249 ± | 0.216 ± |
| | | | 0.001 | 0.001 | 0.001 | 0.001 |
| MESA | 3.39e−07*** | 2.75e−34*** | 0.432 ± | 0.400 ± | 0.418 ± | 0.393 ± |
| | | | 0.001 | 0.001 | 0.001 | 0.001 |
| MESA | 4.80E−01 | 3.13e−04** | 0.681 ± | 0.652 ± | 0.657 ± | 0.646 ± |
| | | | 0.002 | 0.002 | 0.002 | 0.002 |
| MESA | 5.04E−01 | 2.58e−08*** | 0.791 ± | 0.758 ± | 0.766 ± | 0.752 ± |
| | | | 0.001 | 0.002 | 0.002 | 0.002 |
| MESA | 8.15E−01 | 1.07e−12*** | 0.837 ± | 0.796 ± | 0.807 ± | 0.790 ± |
| | | | 0.001 | 0.002 | 0.002 | 0.002 |
| MESA | 8.34E−01 | 2.26e−22*** | 0.840 ± | 0.795 ± | 0.810 ± | 0.789 ± |
| | | | 0.002 | 0.002 | 0.002 | 0.002 |
| MESA | 6.20E−01 | 3.13e−29*** | 0.846 ± | 0.800 ± | 0.819 ± | 0.794 ± |
| | | | 0.002 | 0.002 | 0.002 | 0.002 |
| MESA | 8.51e−66*** | 0.00e+00*** | 0.174 ± | 0.158 ± | 0.152 ± | 0.148 ± |
| | | | 0.001 | 0.001 | 0.001 | 0.001 |
| MESA | 5.95e−08*** | 1.44e−90*** | 0.340 ± | 0.327 ± | 0.301 ± | 0.317 ± |
| | | | 0.001 | 0.001 | 0.002 | 0.001 |
| MESA | 1.54e−03* | 6.53e−53*** | 0.552 ± | 0.542 ± | 0.510 ± | 0.537 ± |
| | | | 0.001 | 0.001 | 0.001 | 0.001 |
| MESA | 2.57E−01 | 2.05e−54*** | 0.759 ± | 0.750? | 0.723 ± | 0.749 ± |
| | | | 0.002 | 0.002 | 0.002 | 0.002 |
| MESA | 6.88e−03* | 2.65e−19*** | 0.828 ± | 0.806 ± | 0.797 ± | 0.805 ± |
| | | | 0.001 | 0.002 | 0.002 | 0.002 |
| MESA | 6.61e−03* | 6.35e−12*** | 0.866 ± | 0.838 ± | 0.834 ± | 0.838 ± |
| | | | 0.002 | 0.002 | 0.002 | 0.002 |
| MESA | 3.34e−02* | 2.46e−02* | 0.864 ± | 0.836 ± | 0.835 ± | 0.834 ± |
| | | | 0.002 | 0.002 | 0.002 | 0.002 |
| MESA | 7.47E−02 | 4.31e−04** | 0.879 ± | 0.847 ± | 0.854 ± | 0.845 ± |
| | | | 0.002 | 0.002 | 0.002 | 0.002 |
| Wellderly | 0.00e+00*** | 5.06e−19*** | 0.212 ± | 0.183 ± | 0.170 ± | 0.170 ± |
| | | | 0.001 | 0.001 | 0.001 | 0.001 |
| Wellderly | 1.44e−39*** | 1.40e−02* | 0.359 ± | 0.307 ± | 0.289 ± | 0.283 ± |
| | | | 0.003 | 0.003 | 0.003 | 0.003 |
| Wellderly | 9.91e−12*** | 1.85e−06*** | 0.616 ± | 0.566 ± | 0.536 ± | 0.544 ± |
| | | | 0.002 | 0.002 | 0.002 | 0.002 |
| Wellderly | 5.76E−01 | 7.95e−12*** | 0.820 ± | 0.783 ± | 0.761 ± | 0.769 ± |
| | | | 0.002 | 0.003 | 0.003 | 0.003 |
| Wellderly | 1.23E−01 | 1.03e−02* | 0.869 ± | 0.830 ± | 0.821 ± | 0.820 ± |
| | | | 0.002 | 0.002 | 0.002 | 0.002 |
| Wellderly | 9.11E−02 | 2.47e−02* | 0.889 ± | 0.856 ± | 0.850 ± | 0.848 ± |
| | | | 0.002 | 0.002 | 0.002 | 0.002 |
| Wellderly | 1.17E−01 | 3.62E−01 | 0.888 ± | 0.851 ± | 0.848 ± | 0.844 ± |
| | | | 0.002 | 0.002 | 0.003 | 0.002 |
| Wellderly | 1.11e−02* | 8.04E−01 | 0.900? | 0.861 ± | 0.859 ± | 0.855 ± |
| | | | 0.002 | 0.002 | 0.002 | 0.002 |
| Wellderly | 2.93e−173*** | 5.08e−13*** | 0.240 ± | 0.188 ± | 0.179 ± | 0.176 ± |
| | | | 0.001 | 0.001 | 0.001 | 0.001 |
| Wellderly | 2.51e−11*** | 3.79E−01 | 0.463 ± | 0.392 ± | 0.374 ± | 0.373 ± |
| | | | 0.003 | 0.003 | 0.003 | 0.003 |
| Wellderly | 3.75e−02* | 1.29e−12*** | 0.761 ± | 0.714 ± | 0.694 ± | 0.701 ± |
| | | | 0.002 | 0.002 | 0.002 | 0.002 |
| Wellderly | 7.27e−05*** | 1.74e−10*** | 0.911 ± | 0.879 ± | 0.868 ± | 0.875 ± |
| | | | 0.001 | 0.002 | 0.002 | 0.002 |
| Wellderly | 2.59e−05*** | 5.92E−01 | 0.930 ± | 0.893 ± | 0.891 ± | 0.889 ± |
| | | | 0.001 | 0.001 | 0.001 | 0.001 |
| Wellderly | 9.63e−04** | 3.48e−05*** | 0.944 ± | 0.902 ± | 0.907 ± | 0.899 ± |
| | | | 0.001 | 0.001 | 0.001 | 0.001 |
| Wellderly | 4.46e−02* | 4.42e−12*** | 0.940 ± | 0.892 ± | 0.899 ± | 0.886 ± |
| | | | 0.001 | 0.002 | 0.002 | 0.002 |
| Wellderly | 1.02e−02* | 6.59e−19*** | 0.949 ± | 0.900 ± | 0.908 ± | 0.895 ± |
| | | | 0.001 | 0.001 | 0.002 | 0.002 |
| Wellderly | 1.06e−207*** | 7.98e−03* | 0.255 ± | 0.215 ± | 0.197 ± | 0.203 ± |
| | | | 0.001 | 0.001 | 0.001 | 0.001 |
| Wellderly | 4.07e−15*** | 1.55e−02* | 0.428 ± | 0.376 ± | 0.347 ± | 0.355 ± |
| | | | 0.003 | 0.003 | 0.003 | 0.003 |
| Wellderly | 1.68e−03* | 7.71e−19*** | 0.706 ± | 0.671 ± | 0.641 ± | 0.653 ± |
| | | | 0.002 | 0.002 | 0.002 | 0.002 |
| Wellderly | 1.12E−01 | 1.08e−31*** | 0.883 ± | 0.862 ± | 0.844 ± | 0.854 ± |
| | | | 0.002 | 0.002 | 0.002 | 0.002 |
| Wellderly | 1.70e−03* | 1.76e−06*** | 0.915 ± | 0.889 ± | 0.885 ± | 0.883 ± |
| | | | 0.002 | 0.002 | 0.002 | 0.002 |
| Wellderly | 3.06e−02* | 6.20E−02 | 0.933 ± | 0.907 ± | 0.904 ± | 0.901 ± |
| | | | 0.001 | 0.002 | 0.002 | 0.002 |
| Wellderly | 1.52E−01 | 3.57e−02* | 0.927 ± | 0.896 ± | 0.897 ± | 0.892 ± |
| | | | 0.002 | 0.002 | 0.002 | 0.002 |
| Wellderly | 1.44e−02* | 2.86e−02* | 0.933 ± | 0.902 ± | 0.904 ± | 0.897 ± |
| | | | 0.002 | 0.002 | 0.002 | 0.002 |
| HGDP | 0.00e+00*** | 4.57e−76*** | 0.115 ± | 0.110 ± | 0.094 ± | 0.097 ± |
| | | | 0.000 | 0.001 | 0.000 | 0.000 |
| HGDP | 2.29e−59*** | 6.90e−31*** | 0.255 ± | 0.245 ± | 0.212 ± | 0.224 ± |
| | | | 0.001 | 0.001 | 0.001 | 0.001 |
| HGDP | 1.46e−68*** | 3.88e−09*** | 0.477 ± | 0.461 ± | 0.416 ± | 0.428 ± |
| | | | 0.001 | 0.001 | 0.001 | 0.001 |
| HGDP | 3.24e−32*** | 4.07e−02* | 0.720 ± | 0.695 ± | 0.662 ± | 0.660 ± |
| | | | 0.002 | 0.002 | 0.002 | 0.002 |
| HGDP | 1.49e−33*** | 4.55E−01 | 0.791 ± | 0.765 ± | 0.747 ± | 0.733 ± |
| | | | 0.002 | 0.002 | 0.002 | 0.002 |
| HGDP | 2.08e−20*** | 6.08E−02 | 0.813 ± | 0.786 ± | 0.774 ± | 0.760 ± |
| | | | 0.002 | 0.002 | 0.002 | 0.002 |
| HGDP | 9.49e−21*** | 7.35E−02 | 0.835 ± | 0.806 ± | 0.793 ± | 0.782 ± |
| | | | 0.002 | 0.002 | 0.002 | 0.002 |
| HGDP | 3.98e−16*** | 7.45e−05*** | 0.836 ± | 0.805 ± | 0.797 ± | 0.781 ± |
| | | | 0.002 | 0.002 | 0.002 | 0.002 |
| HGDP | 0.00e+00*** | 1.59e−03* | 0.109 ± | 0.096 ± | 0.086 ± | 0.080 ± |
| | | | 0.000 | 0.000 | 0.000 | 0.000 |
| HGDP | 4.56e−76*** | 5.59E−01 | 0.233 ± | 0.206 ± | 0.193 ± | 0.180 ± |
| | | | 0.001 | 0.001 | 0.001 | 0.001 |
| HGDP | 2.07e−107*** | 1.88e−12*** | 0.455 ± | 0.421 ± | 0.396 ± | 0.382 ± |
| | | | 0.001 | 0.001 | 0.001 | 0.001 |
| HGDP | 9.53e−57*** | 2.15e−24*** | 0.746 ± | 0.718 ± | 0.704 ± | 0.678 ± |
| | | | 0.002 | 0.002 | 0.002 | 0.002 |
| HGDP | 9.55e−77*** | 3.82e−55*** | 0.817 ± | 0.785 ± | 0.779 ± | 0.748 ± |
| | | | 0.001 | 0.001 | 0.001 | 0.002 |
| HGDP | 2.43e−55*** | 2.29e−60*** | 0.844 ± | 0.808 ± | 0.805 ± | 0.773 ± |
| | | | 0.001 | 0.002 | 0.002 | 0.002 |
| HGDP | 2.63e−52*** | 3.67e−70*** | 0.860 ± | 0.819 ± | 0.818 ± | 0.788 ± |
| | | | 0.001 | 0.002 | 0.002 | 0.002 |
| HGDP | 2.88e−45*** | 3.80e−83*** | 0.861 ± | 0.821 ± | 0.827 ± | 0.791 ± |
| | | | 0.001 | 0.002 | 0.002 | 0.002 |
| HGDP | 3.78e−302*** | 1.17e−153*** | 0.139 ± | 0.135 ± | 0.112 ± | 0.117 ± |
| | | | 0.001 | 0.001 | 0.001 | 0.001 |
| HGDP | 2.71e−56*** | 1.73e−59*** | 0.292 ± | 0.290 ± | 0.240 ± | 0.261 ± |
| | | | 0.001 | 0.001 | 0.001 | 0.001 |
| HGDP | 3.08e−85*** | 2.14e−34*** | 0.513 ± | 0.510 ± | 0.451 ± | 0.474 ± |
| | | | 0.001 | 0.001 | 0.001 | 0.001 |
| HGDP | 2.87e−45*** | 2.85e−05*** | 0.772 ± | 0.764 ± | 0.730 ± | 0.730 ± |
| | | | 0.002 | 0.002 | 0.002 | 0.002 |
| HGDP | 6.73e−57*** | 5.16e−03* | 0.822 ± | 0.805 ± | 0.787 ± | 0.774 ± |
| | | | 0.001 | 0.002 | 0.002 | 0.002 |
| HGDP | 5.09e−43*** | 7.27e−05*** | 0.851 ± | 0.832 ± | 0.819 ± | 0.805 ± |
| | | | 0.002 | 0.002 | 0.002 | 0.002 |
| HGDP | 4.34e−44*** | 4.80e−07*** | 0.873 ± | 0.848 ± | 0.836 ± | 0.821 ± |
| | | | 0.001 | 0.002 | 0.002 | 0.002 |
| HGDP | 2.67e−37*** | 1.11e−23*** | 0.867 ± | 0.843 ± | 0.837 ± | 0.817 ± |
| | | | 0.002 | 0.002 | 0.002 | 0.002 |

Validation accuracies were stratified by dataset (MESA, Wellderly, HGDP), genotype array platform (Affymetrix 6.0, UKB Axiom, Omni 1.5M), and MAF bin.
Wilcoxon rank-sum tests were applied to compare the HMM-based tools to the referencetuned autoencoder (AE).
*represents p-values ≤ 0.05,
**indicates p-values ≤ 0.001, and
***indicates p-values ≤ 0.0001.

### Ancestry-Specific Chromosome 22 Imputation Accuracy.

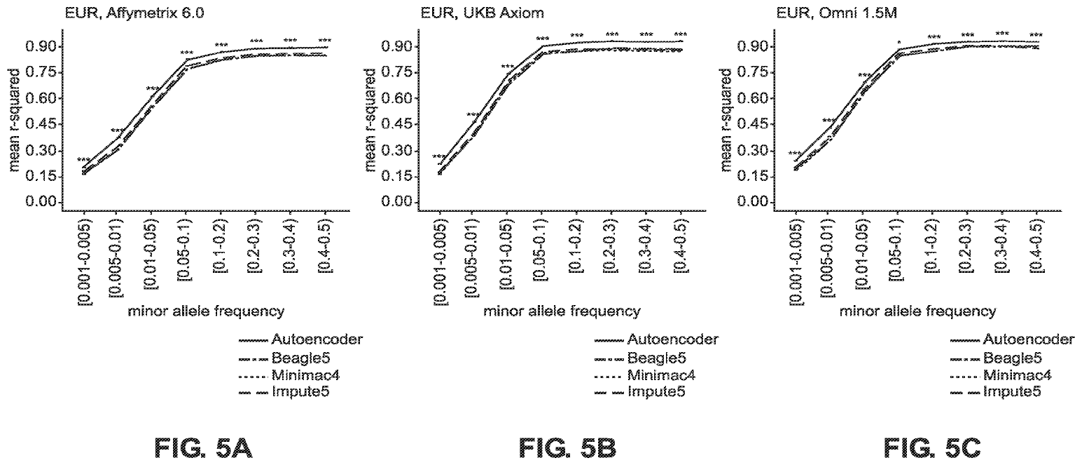

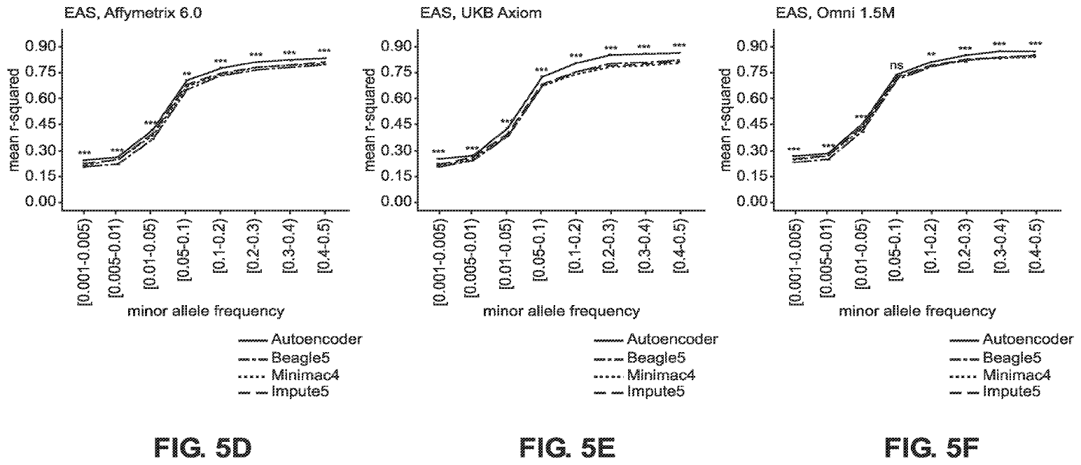

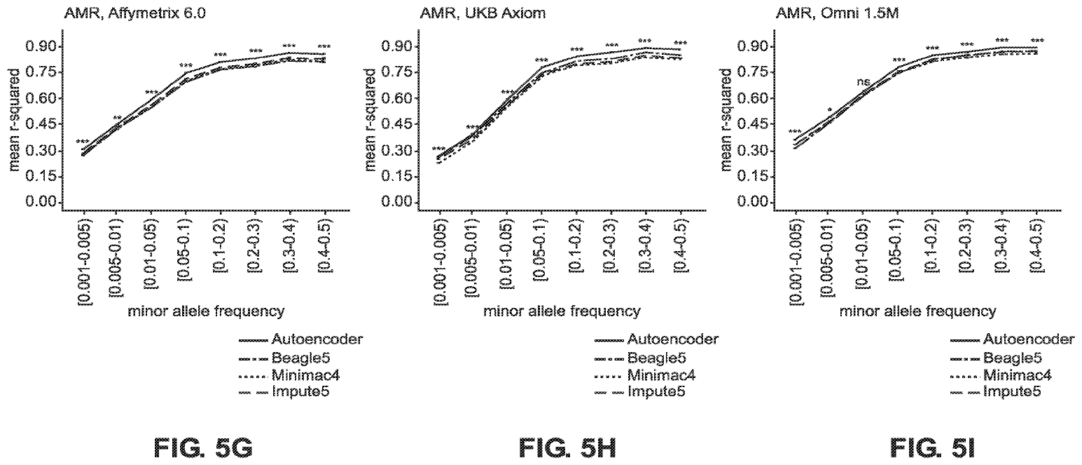

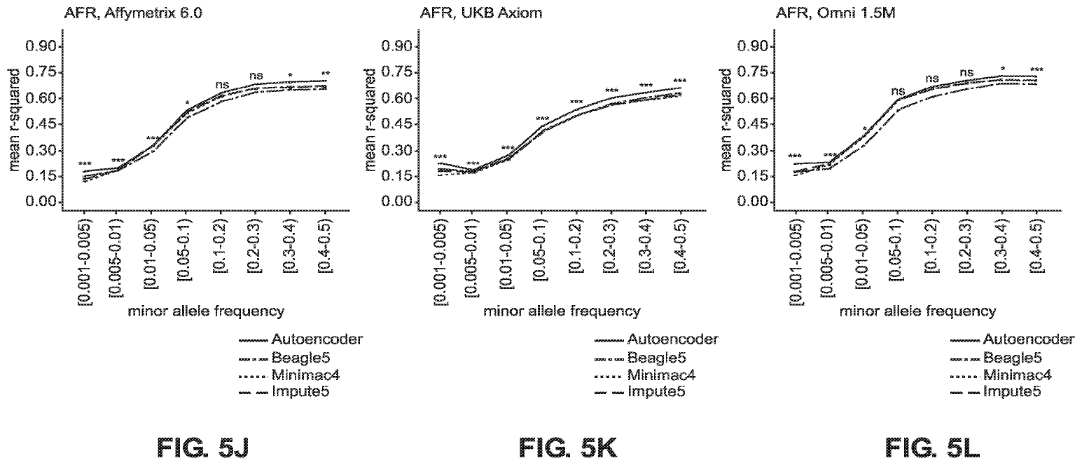

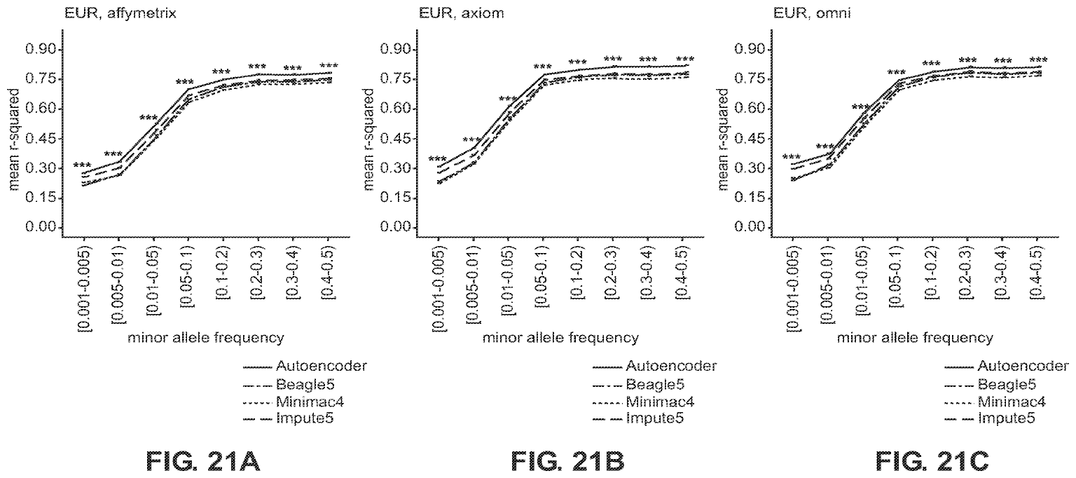

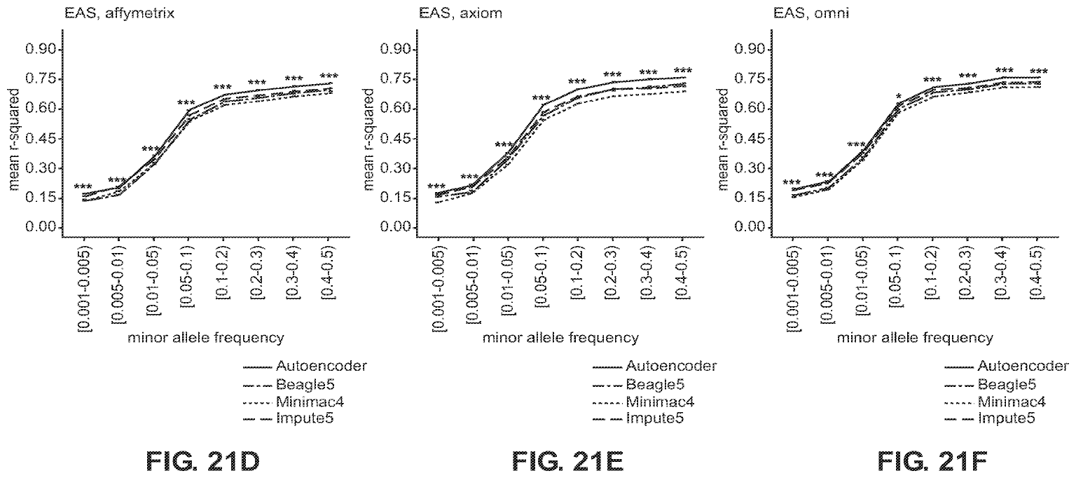

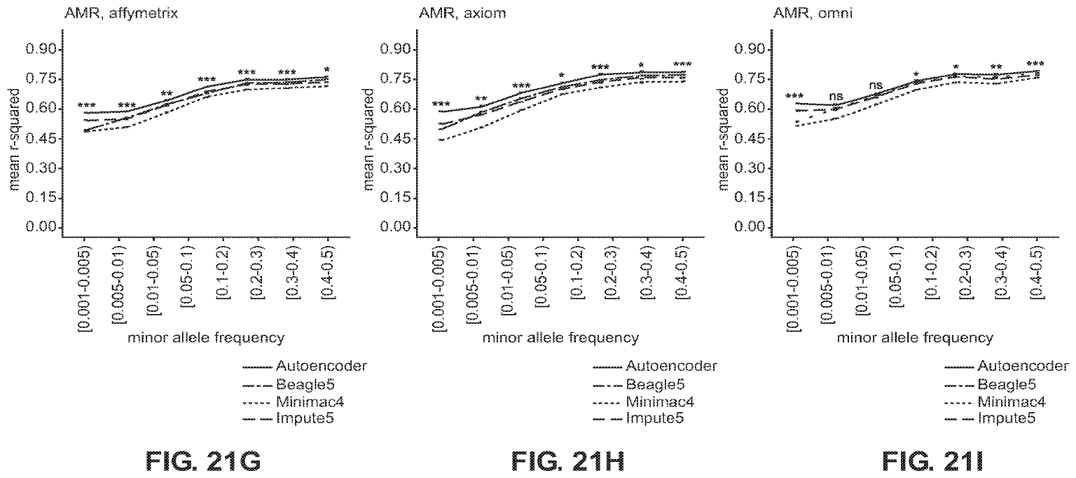

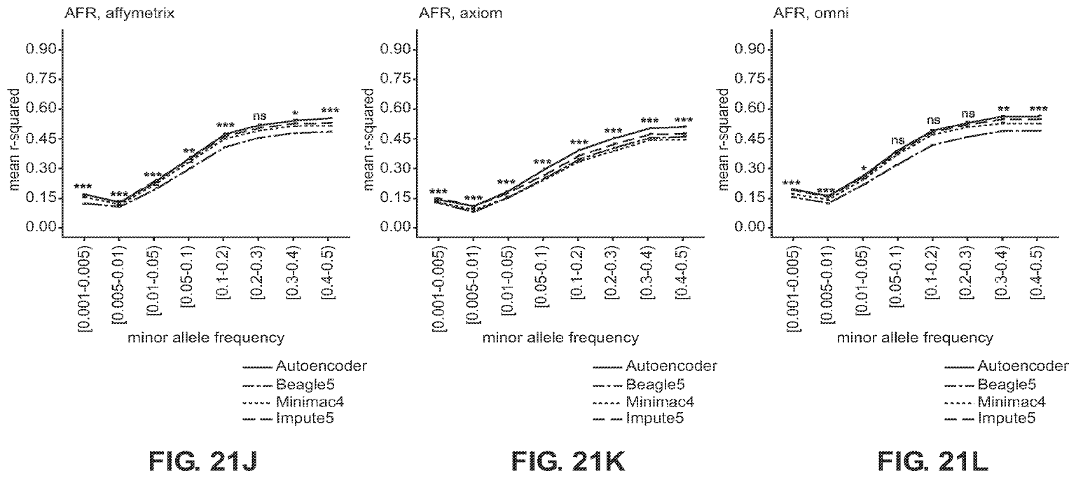

Finally, the inventors evaluated ancestry-specific imputation accuracy. As before, overall autoencoder-based imputation maintains superiority across all continental populations present in MESA (FIG. 5, Wilcoxon rank-sums test p=5.39×10⁻¹⁹). The autoencoders' mean r-squared ranged from 0.357 for African ancestry to 0.614 for East Asian ancestry vs 0.328 to 0.593 for Minimac4, 0.330 to 0.544 for Beagle5, and 0.324 to 0.586 for Impute5, respectively. Note, East Asian ancestry exhibits a slightly higher overall imputation accuracy relative to European ancestry due to improved rare variant imputation. Autoencoder superiority replicates when HGDP is split into continental populations (FIG. 21).

Further stratification of ancestry-specific imputation accuracy results by MAF continues to support autoencoder superiority across all ancestries, MAF bins, and nearly all test datasets, and genotyping array marker sets (FIG. 5, FIG. 21). Minimum and maximum accuracies across MAF by ancestry bins ranged between 0.177 to 0.937 for the autoencoder, 0.132 to 0.907 for Minimac4, 0.147 to 0.909 for Beagle5, and 0.115 to 0.903 for Impute5, with a maximum standard error of +0.004.

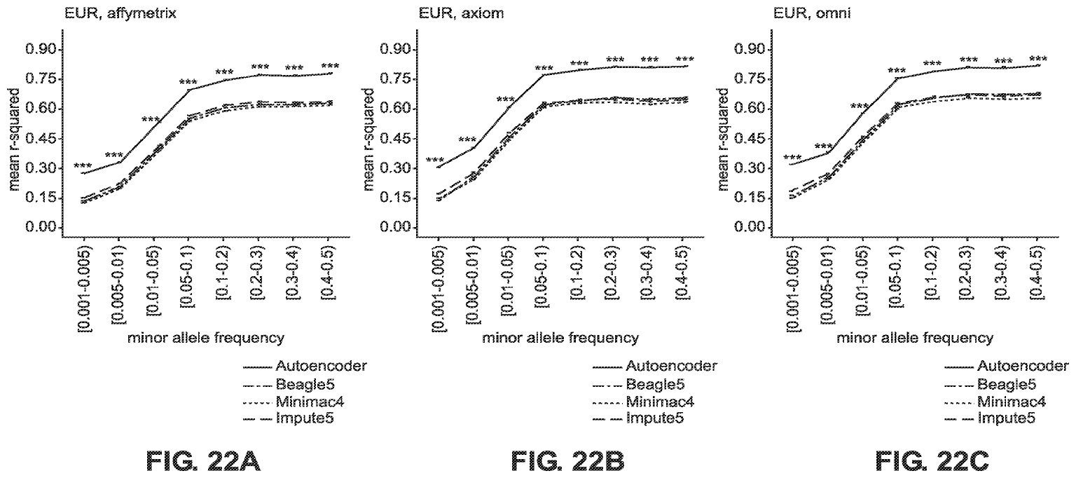

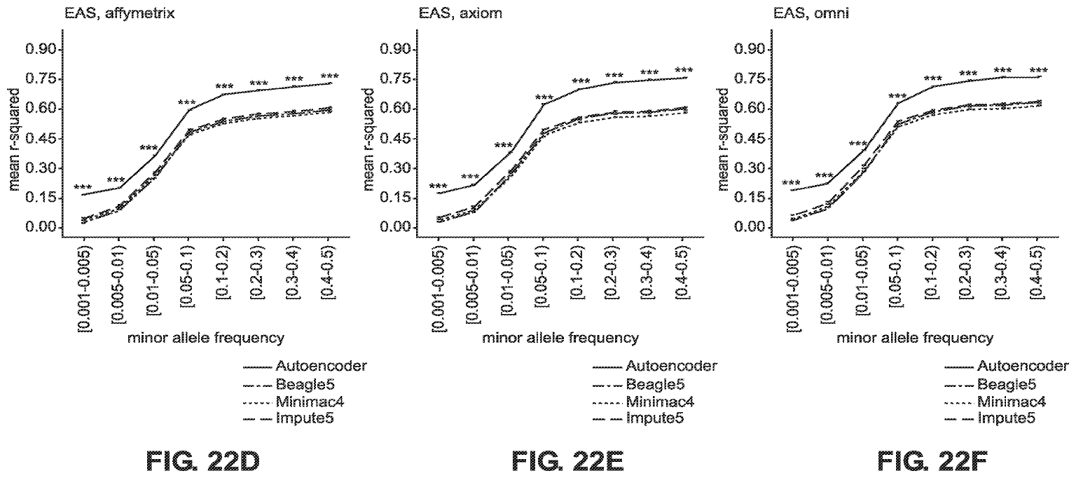

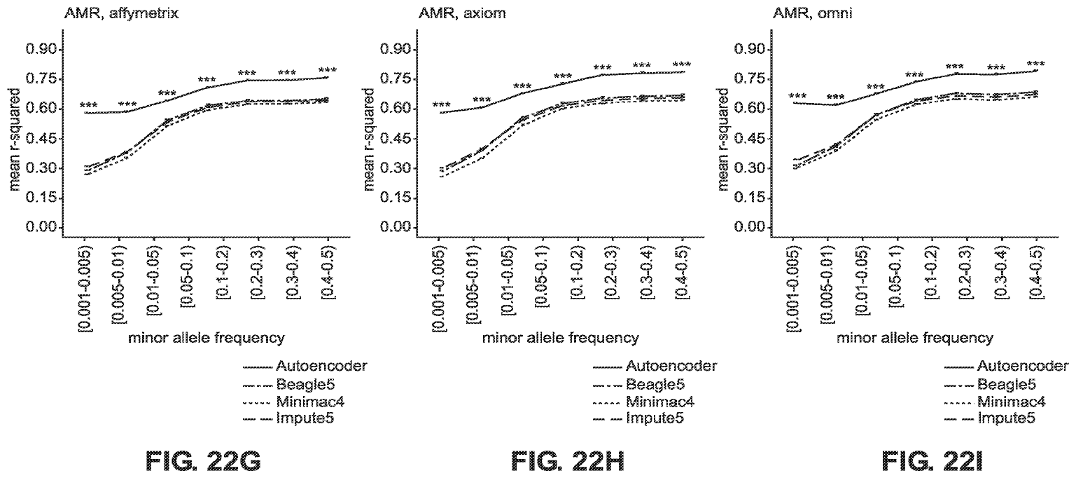

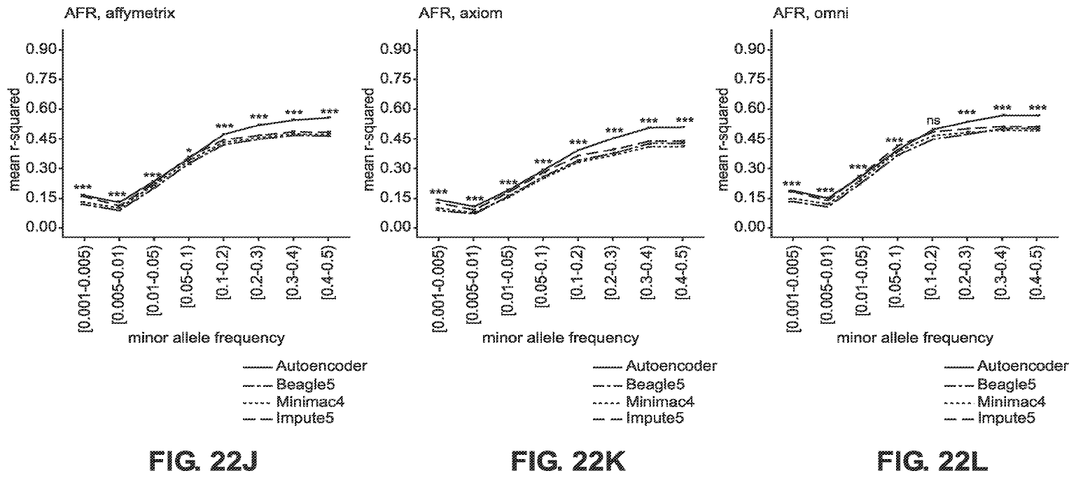

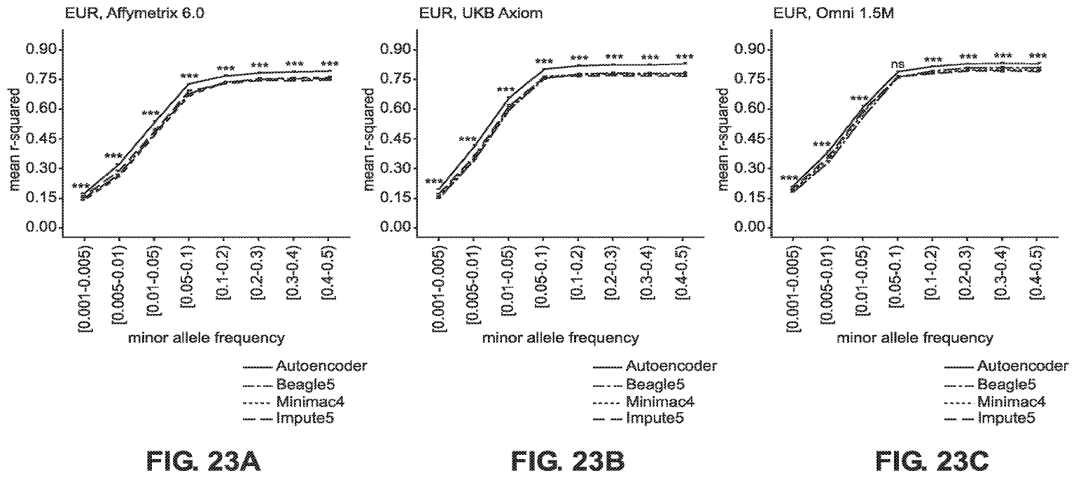

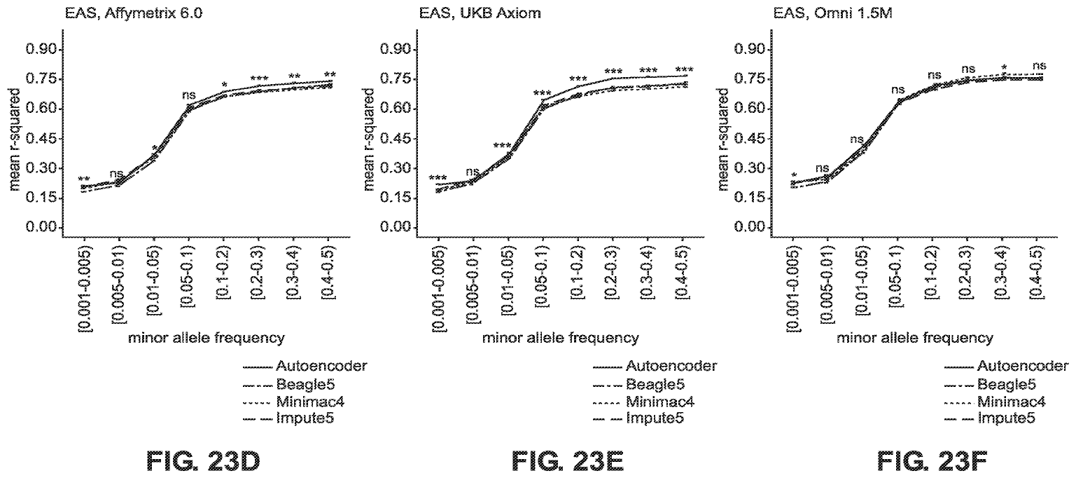

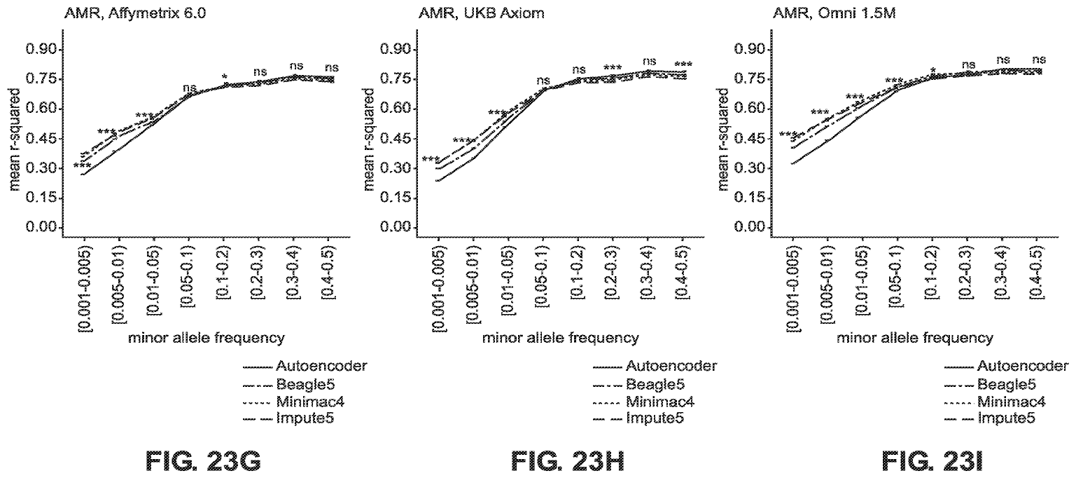

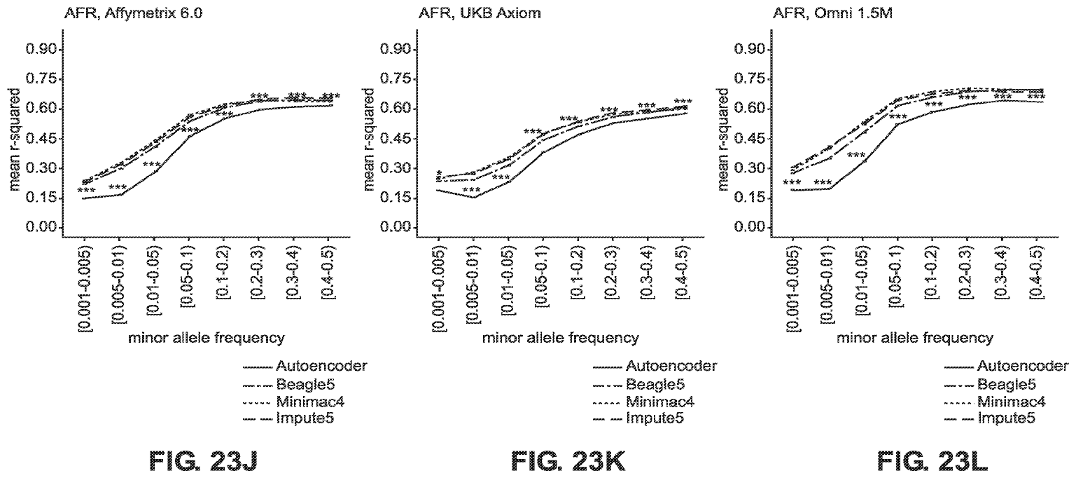

Thus, with training on equivalent reference cohorts, autoencoder performance was superior across all variant allele frequencies and ancestries with the primary source of superiority arising from hard to impute regions with complex LD structures. When the reference panel of the HMM-based tools is upgraded to the more diverse TOPMed dataset, the HRC-trained autoencoder remains superior across all ancestry groups of HGDP (FIG. 5 and FIG. 22), as well as in the MESA ancestries well represented in HRC (European and East Asian) but not in MESA ancestries where representation is significantly enhanced by the TOPMed reference panel (American and African) with additional imputation performance deriving from a significant degree of familial relationships spanning the TOPMed reference panel and MESA test cohort (FIG. 23).

### Inference Speed.

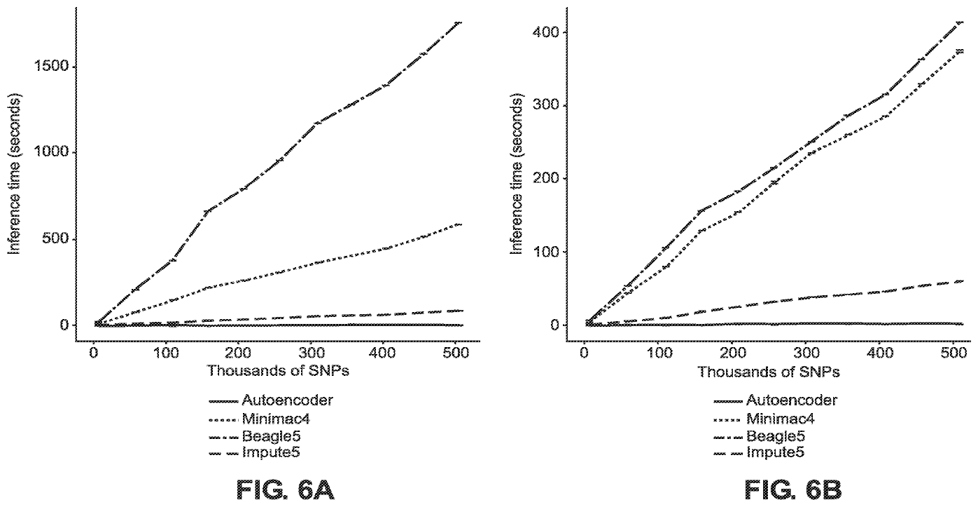

Inference runtimes for the autoencoder vs HMM-based methods were compared in a low-end and high-end computational environment as described herein. In the low-end environment, the autoencoder's inference time is at least ˜4× faster than all HMM-based inference times (summing all inference times from all genomic segments of chromosome 22, the inference time for the autoencoder was 2.4±1.1*103 seconds versus 1,754±3.2, 583.3±0.01, and 8.4±4.3*10-3 seconds for Minimac4, Beagle5, and Impute5, respectively (FIG. 6A). In the high-end environment, this difference narrows to a ˜3× advantage of the autoencoder vs HMM-based methods (2.1±8.0*10-4 versus 374.3±1.2, 414.3±0.01, and 6.1±2.1*10-4 seconds for Minimac4, Beagle5, and Impute5, respectively (FIG. 6B). These unoptimized results indicate that autoencoder-based imputation can be executed rapidly, without a reference cohort, and without the need for a high-end server or high-performance computing (HPC) infrastructure.

### Example 2

Artificial neural network-based data mining techniques are revolutionizing biomedical informatics and analytics35,36. Here, the inventors have demonstrated the potential for these techniques to execute a fundamental analytical task in population genetics, genotype imputation, producing superior results in a computational efficient and portable framework. The trained autoencoders can be transferred easily, and execute their functions rapidly, even in modest computing environments, obviating the need to transfer private genotype data to external imputation servers or services. Furthermore, the fully trained autoencoders robustly surpass the performance of all modern HMM-based imputation approaches across all tested independent datasets, genotyping array marker sets, minor allele frequency spectra, and diverse ancestry groups. This superiority was most apparent in genomic regions with low LD and/or high complexity in their linkage disequilibrium structure.

Superior imputation accuracy is expected to improve GWAS power, enable more complete coverage in meta-analyses, and improve causal variant identification through fine-mapping. Moreover, superior imputation accuracy in low LD regions may enable the more accurate interrogation of specific classes of genes under a greater degree of selective pressure and involved in environmental sensing. For example, promoter regions of genes associated with inflammatory immune responses, response to pathogens, environmental sensing, and neurophysiological processes (including sensory perception genes) are often located in regions of low LD 36,37. These known disease-associated biological processes that are critical to interrogate accurately in GWAS. Thus, the autoencoder-based imputation approach both improves statistical power and biological coverage of individual GWAS' and downstream meta-analyses.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Li, Y., Willer, C., Sanna, S. & Abecasis, G. Genotype Imputation. Annual Review of Genomics and Human Genetics 10, 387-406 (2009).
2. Marchini, J. & Howie, B. Genotype imputation for genome-wide association studies. Nature Reviews Genetics vol. 11 499-511 (2010).
3. Das, S. et al. Next-generation genotype imputation service and methods. Nature Genetics 48, 1284-1287 (2016).
4. Rubinacci, S., Delaneau, O. & Marchini, J. Genotype imputation using the Positional Burrows Wheeler Transform. PLOS Genetics 16, e1009049 (2020).
5. Browning, B. L., Zhou, Y. & Browning, S. R. A One-Penny Imputed Genome from Next-Generation Reference Panels. American Journal of Human Genetics 103, 338-348 (2018).
6. Browning, B. L. & Browning, S. R. Genotype Imputation with Millions of Reference Samples. American Journal of Human Genetics 98, 116-126 (2016).
7. Das, S., Abecasis, G. R. & Browning, B. L. Genotype Imputation from Large Reference Panels. Annual Review of Genomics and Human Genetics 19, 73-96 (2018).
8. Kowalski, M. H. et al. Use of >100,000 NHLBI Trans-Omics for Precision Medicine (TOPMed) Consortium whole genome sequences improves imputation quality and detection of rare variant associations in admixed African and Hispanic/Latino populations. PLOS Genetics 15, e1008500 (2019).
9. Sarkar, E. et al. Fast and scalable private genotype imputation using machine learning and partially homomorphic encryption. IEEE Access (2021).
10. Lal, A. et al. Deep learning-based enhancement of epigenomics data with AtacWorks. Nature Communications 12, (2021).
11. Arisdakessian, C., Poirion, O., Yunits, B., Zhu, X. & Garmire, L. X. DeepImpute: An accurate, fast, and scalable deep neural network method to impute single-cell RNA-seq data. Genome Biology 20, 211 (2019).
12. Koh, P. W., Pierson, E. & Kundaje, A. Denoising genome-wide histone ChIP-seq with convolutional neural networks. Bioinformatics 33, i225-i233 (2017).
13. Abouzid, H., Chakkor, O., Reyes, O. G. & Ventura, S. Signal speech reconstruction and noise removal using convolutional denoising audioencoders with neural deep learning. Analog Integrated Circuits and Signal Processing 100, 501-512 (2019).
14. Liu, Y. et al. Multilingual Denoising Pre-training for Neural Machine Translation. Transactions of the Association for Computational Linguistics 8, 726-742 (2020).
15. Voulodimos, A., Doulamis, N., Doulamis, A. & Protopapadakis, E. Deep Learning for Computer Vision: A Brief Review. Computational Intelligence and Neuroscience vol. 2018 (2018).
16. Tian, C. et al. Deep learning on image denoising: An overview. Neural Networks vol. 131 251-275 (2020).
17. Naito, T. et al. A deep learning method for HLA imputation and trans-ethnic MHC fine-mapping of type 1 diabetes. Nature Communications 12, 1-14 (2021).
18. Chen, J. & Shi, X. Sparse Convolutional Denoising Autoencoders for Genotype Imputation. Genes 10, 652 (2019).
19. Islam, T. et al. A Deep Learning Method to Impute Missing Values and Compress Genome-ide Polymorphism Data in Rice. in Proceedings of the 14th International Joint Conference on Biomedical Engineering Systems and Technologies, {BIOSTEC} 2021, Volume 3: BIOINFORMATICS, Online Streaming, Feb. 11-13, 2021 (eds. Lorenz, R., Fred, A. L. N. & Gamboa, H.) 101-109 (SCITEPRESS, 2021).
20. Kojima, K. et al. A genotype imputation method for de-identified haplotype reference information by using recurrent neural network. PLOS Computational Biology 16, e1008207 (2020).
21. Sun, Y. v. & Kardia, S. L. R. Imputing missing genotypic data of single-nucleotide polymorphisms using neural networks. European Journal of Human Genetics 16, 487-495 (2008).
22. Auton, A. et al. A global reference for human genetic variation. Nature vol. 526 68-74 (2015).
23. McCarthy, S. et al. A reference panel of 64,976 haplotypes for genotype imputation. Nature Genetics 48, 1279-1283 (2016).
24. Alexander, D. H., Novembre, J. & Lange, K. Fast model-based estimation of ancestry in unrelated individuals. Genome Research 19, 1655-1664 (2009).
25. Mou, L. et al. Lifetime Risk of Atrial Fibrillation by Race and Socioeconomic Status: ARIC Study (Atherosclerosis Risk in Communities). Circulation: Arrhythmia and Electrophysiology 11, (2018).
26. Erikson, G. A. et al. Whole-Genome Sequencing of a Healthy Aging Cohort. Cell 165, 1002-1011 (2016).
27. Cann, H. M. A Human Genome Diversity Cell Line Panel. Science 296, 261b-2262 (2002).
28. Bild, D. E. Multi-Ethnic Study of Atherosclerosis: Objectives and Design. American Journal of Epidemiology 156, 871-881 (2002).
29. Picard toolkit. (2019).
30. Danecek, P. et al. Twelve years of SAMtools and BCFtools. GigaScience 10, 1-4 (2021).
31. Dimitromanolakis, A., Xu, J., Krol, A. & Briollais, L. sim1000G: A user-friendly genetic variant simulator in R for unrelated individuals and family-based designs. BMC Bioinformatics 20, 26 (2019).
32. Lin, T.-Y., Goyal, P., Girshick, R., He, K. & Dollár, P. Focal Loss for Dense Object Detection. (2017).
33. Berisa, T. & Pickrell, J. K. Approximately independent linkage disequilibrium blocks in human populations. Bioinformatics 32, 283-285 (2016).
34. Das, S. et al. Next-generation genotype imputation service and methods. Nature Genetics 48, 1284-1287 (2016).
35. Jumper, J. et al. Highly accurate protein structure prediction with AlphaFold. Nature 1-7 (2021) doi: 10.1038/s41586-021-03819-2.
36. Dias, R. & Torkamani, A. Artificial intelligence in clinical and genomic diagnostics. Genome Medicine vol. 11 (2019).
37. Frazer, K. A. et al. A second generation human haplotype map of over 3.1 million SNPs. Nature 449, 851-861 (2007).
38. Okewu, E., Adewole, P. & Sennaike, O. Experimental Comparison of Stochastic Optimizers in Deep Learning. in Lecture Notes in Computer Science (including subseries Lecture Notes in Artificial Intelligence and Lecture Notes in Bioinformatics) vol. 11623 LNCS 704-715 (Springer Verlag, 2019).

What is claimed is:

1. A system for dynamically producing predictive data using varying data, wherein the system is structured to generate imputed genomic sequence by inputting actual genomic sequence into an artificial neural network, the system comprising:
a. an artificial neural network;
b. a communication device;
c. a processing device communicably coupled to the communication device, wherein the processing device is configured to:
(i) receive actual genetic information;
(ii) generate an input vector, wherein the input vector comprises probabilistic or binary data converted from allelic data;
(iii) access the neural network comprising a dynamic reservoir containing units, wherein each of the units is connected to at least one other unit in the dynamic reservoir and the connections between the units are weighted;
(iv) input the input vector into the neural network in order to generate an imputed sequence;
(vi) generate, via the neural network, an imputed sequence, wherein the imputed sequence is an output of the neural network and at least partially based on the input vector;
(vii) modify the initial prediction to generate a final prediction;
(viii) present the final prediction to a user; and
(ix) export the final prediction to a computer system.

2. The system of claim 1, wherein the variables comprise data relating to at least one genetic variant.

3. The system of claim 1, wherein the genetic variant is at least one of: a single-nucleotide variant, a multi-nucleotide variant, an insertion, a deletion, a structural variation, an inversion, a copy-number change, or any other genetic variation relative to a reference genome.

4. The system of claim 1, wherein the units comprise nodes within at least three layers.

5. The system of claim 1, wherein the dynamic reservoir comprises a plurality of layers.

6. The system of claim 1, wherein the at least three layers comprise an input layer, a hidden layer, and an output layer.

7. The system of claim 1, wherein the system comprises a plurality of dynamic reservoirs, wherein each reservoir is adapted to impute sequence of a selected portion of genetic data, and wherein the plurality of reservoirs is adapted to cover all desired portions of a genome or fragment thereof.

8. The system of claim 1, wherein the initial prediction is provided in binary form, and the final prediction is provided as genetic sequence.

9. A method of obtaining, from an input of incomplete genomic information from an individual or population of an organism, an output of more complete genomic information for the individual or population within a desired accuracy cutoff, comprising the steps of:
(a) providing a genetic inference model comprising encoded complex genotype relationships, said model having been encoded by the system of claim 1,
(b) inputting incomplete genomic information from the individual or population into the model in or mediated by the system wherein the incomplete genetic information comprises at least a sparse genotyping or sequencing, as defined and described herein, of a randomly sampled genome of the individual or population;
(c) applying the model to the information by operation of the system; and
(d) obtaining the output of more complete genomic information for the individual or population, wherein the more complete genomic information comprises genotypes for genetic variants observed in a reference population used to define the weights of the neural network.

10. The method of claim 9, wherein the accuracy cutoff is an accuracy level as in any of the ranges depicted in any of the figures, or any other useful accuracy.

11. The method of claim 9, wherein the organism is selected from an animal, a plant, a fungus, a chromistan, a protozoan, a bacterium, and an aracheon.

12. A method of training the system of claim 1, comprising the steps of:
(a) providing training genetic sequence data;
(b) generating a first training input vector, wherein the first training input vector comprises binary or probabilistic data converted from allelic data of the training genetic sequence data;
(c) generating a second training input vector from the first training input vector, wherein the second input vector comprises reducing a number of variables of the first training input vector;
(d) inputting the second training input vector to a neural network comprising a dynamic reservoir containing units, wherein each of the units is connected to at least one other unit in the dynamic reservoir and wherein the connections between the units are initially weighted according to a training weighting;
(e) generating, via the neural network, imputed training sequence data, wherein the imputed training sequence data are an output of the neural network and at least partially based on the second input vector;
(f) comparing the imputed training sequence data from step (e) to the original training genetic sequence data from step (a) to obtain a training result reflecting a degree of correspondence between the imputed training sequence data and the original training genetic sequence data;
(g) modifying the at least one of the generating of step (c) and the weighting of step (d), based upon the training result of step (f);
(h) repeating steps (c) through (g) until the correspondence reaches a predetermined value;
and thereby
(i) completing training of the system.

13. The method of claim 12, wherein the training genetic sequence comprises at least one full genomic sequence of an organism.

14. The method of claim 12, wherein the training genetic sequence comprises a plurality of full genomic sequences from a selected population of an organism.

15. The method of claim 12, wherein the organism is selected from an animal, a plant, a fungus, a chromistan, a protozoan, a bacterium, and an aracheon.
